(19) United States
(12) Patent Application Publication
(10) Pub. No.: US 2025/0027938 A1
(43) Pub. Date: Jan. 23, 2025

(54) METABOLIC SIGNATURE FOR PREDICTION OF MUCOSAL INFLAMMATION AND MICROBIAL COMPOSITION

(71) Applicant: IMPERIAL COLLEGE INNOVATIONS LIMITED, London (GB)

(72) Inventors: David A. MACINTYRE, London (GB); Pamela PRUSKI, London (GB); Gonçalo DOS SANTOS CORREIA, London (GB); Holly LEWIS, London (GB); Phillip R. BENNETT, London (GB); Zoltan TAKATS, London (GB)

(21) Appl. No.: 18/578,462

(22) PCT Filed: Jul. 15, 2022
(86) PCT No.: PCT/GB2022/051833

§ 371 Date: Jan. 11, 2024

(30) Foreign Application Priority Data
Jul. 16, 2021 (GB) 2110293.4

(51) Int. Cl. G01N 33/569; G01N 33/68

(52) U.S. Cl. CPC G01N 33/56911; G01N 33/6848

(57) ABSTRACT

The present invention relates to methods for obtaining a metabolic signature in a subject, said method being applicable for a range of purposes, including diagnosing disease, monitoring disease progression and/or determining a suitable treatment plan.

### CROSS REFERENCE TO RELATED APPLICATIONS

This application is a National Stage application of International Application No. PCT/GB2022/051833, filed Jul. 15, 2022 This application also claims priority under 35 U.S.C. § 119 to United Kingdom Patent Application No. 2110293.4, filed Jul. 16, 2021.

### REFERENCE TO SEQUENCE LISTING SUBMITTED VIA PATENT CENTER

This application was filed electronically via Patent Center and includes an electronically submitted sequence listing in XML format. The XML file contains a sequence listing entitled “2024 Jul. 29 Revised Sequence Listing 86372-400025.xml” created on Jul. 25, 2024, and is 15.9 kb in size. The sequence listing contained in this XML file is part of the specification and is hereby incorporated by reference herein in its entirety.

The project leading to this application has received funding from the European Research Council (ERC) under the European Union's Horizon 2020 research and innovation programme (grant agreement No 825813).

### FIELD OF THE INVENTION

The present invention relates to methods for obtaining a metabolic signature in a subject, said method being applicable for a range of purposes, including diagnosing disease, monitoring disease progression and/or determining a suitable treatment plan.

### BACKGROUND OF THE INVENTION

Various microbes are natural inhabitants of mucosal membranes found in the human body, and are essential for maintaining the balance between “good” microbes and “bad” microbes. One such example of this is the mucosal membrane of the vagina. However, others may cause infection and disease (e.g. bacterial vaginosis, thrush, Group B streptococcus).

During pregnancy, these infections can cause miscarriage, preterm birth and additionally infect newborns in utero or at delivery. Currently, diagnostic tests for vaginal infections (e.g. Gram staining, Amsel criteria, Nugent scoring) rely on subjective interpretation of clinical symptoms and the use of standard medical swabs followed by laboratory-based culture and microscopy to identify pathogenic microbes in vaginal swab samples. These approaches are inherently subjective and inaccurate as certain bacteria grow faster than others and many cannot be cultured at all. Microscopy-based analyses are limited to overly simplistic evaluation of cell shape and number. Commercially available assays that work by measuring the concentrations of specific enzymes produced by microbes in the vagina can only be used for the rapid diagnosis of certain vaginal infections, such as yeast infection or bacterial vaginosis. Additionally, these methods fail to consider the interplay with the subject's immune system, which are critical in understanding the origin and cause of pathology and disease, and thus the selection of the most appropriate treatments.

Currently, the diagnosis of inflammation or immune activation requires collection of additional samples and the undertaking of addition expensive, time-consuming tests and assays.

### SUMMARY OF THE INVENTION

The present invention addresses many of these problems with known techniques and enables the simultaneous measurement of both vaginal microbes and immune responses within the same clinical sample. The use of specific mass-spectrometry (MS) approaches (e.g. Desorption electrospray ionisation-MS) enables the chemical signatures used in the invention to be obtained in approximately 1 min. This makes the invention amenable to point-of-care, bedside use. This represents a significant improvement on known uses enabling faster and more accurate diagnosis at the bedside, allowing clinicians to treat patients sooner and thus improving outcomes.

In a first aspect, the present invention provides a method comprising the steps of: (i) analysing a mucosal sample obtained from a subject to identify the presence and/or level of one or more biomarkers, or any combination of the biomarkers, wherein the biomarkers are selected from a panel of biomarkers, wherein the panel of biomarkers comprises amino-aminohexyl-diaminomethylideneamino-pentanamide, aminonaphthalene, amino-sulfanylheptenyl-amino-oxooctadecanoic acid, arachidic acid (C20:0), arginine, asparagine, aspartic acid, butynoate, cadaverine, carboxysulfanyl-pentanedioic acid, Cer(d24:1/18:1), cerotic acid (C26:0), DG(36:3), dihydroxy-octadecadienoic acid, dimethoxyphenyl-hexadecylcarbamothioyl-propenamide, docosanoic acid (C22:0), docosanylsulfanylbutanoic acid, docosenoic acid (C22:1), eicosadienoic acid (C20:2), eicosanoic acid, ethyl-hexadecylcarbamothioylamino-oxobutanoate, galabiosylceramide (d18:1/16:0), galbeta-Cer(d40:1), glutamate, glutamine, glutamyl-leucine, glycyl-phenylalanine, heptadecylenic acid (C17:1), heptadecyl-hydroxyimidazole, hexadecenylsuccinic acid, histamine, HODE, hydroxycaproic acid, hydroxyglutaric acid, hydroxyvaleric acid, lactoylcysteine, leucine, leucyl-alanine, leucyl-glutamine, leucyl-glycyl-glycine, leucyl-serine, leucyl-threonine, lignoceric acid (C24:0), linoleic acid (C18:2), linolenic acid (C18:3), lysine, lyso-PE(16:1), lyso-PE(18:1), lyso-PE(18:2), lyso-PE(20:4), lyso-PE(O-16:0), lyso-PG(16:0), lyso-PS(18:0), maltotriose, methoxymethyl methyl-glucopyranosiduronate, methyl-hexadecylcarbamothioylamino-oxobutanoate, N2-acetyl-ornithine, N-acetylcadaverine, N-acetylputrescine, nervonic acid (C24:1), nonadecanoic acid (C19:0), nonadecenoic acid (C19:1), N-palmitoyl glutamine, N-palmitoyl methionine, N-palmitoyl-cysteine, octadecanoyloxamide, octadecanyloxy-propoxy-thiazolyl-propanamide, octadecenylamino-sulfanylpropanoic acid, octadecyl-oxy-octadecanoic acid (SAHSA), oleic acid (C18:1), oxo-octadecadienoic acid, oxoproline, oxosulfanyl-oxazolidinyl-octadecanamide, oxypurinol, PC(O-14:1/18:2), pentacosanoic acid (C25:0), PG(18:1/18:2), phenyl sulfoxide, phenylalanine, phenylethylamine, phenyllactic acid, phosphatidyl glycerol, phytenic acid (C20:1), PI(20:4/18:0), propyl-hexadecylcarbamothioylamino-oxobutanoate, PS(16:0/18:1), PS(18:0/18:1), putrescine, pyridinamine, pyrrolidinecarboxaldehyde, stearic acid (18:0), styrene, succinic acid, thiomalic acid, tolualdehyde, tricosanoic acid (C23:0), tyramine, vaccenyl carnitine, valyl-alanine, valyl-glutamine, valyl-phenylalanine, valyl-serine, vinylaniline, and ximenic acid (C26:1); (ii) forming a metabolic profile, wherein said metabolic profile is formed from data showing the presence and/or level of the biomarkers identified in step (i); (iii) using a predictive multivariate statistical model and/or a machine learning model pre-trained with metabolic and immuno-profiling datasets obtained from a reference population to predict the subject's mucosal inflammatory state at a mucosal membrane and/or level of immune activation at a mucosal membrane; and wherein the method further comprises the step of associating the metabolic profile of step ii) with metataxonomic data from a reference population to obtain the microbial composition of the mucosal sample.

In a second aspect, the present invention provides a method of monitoring the mucosal inflammatory state, level of mucosal immune activation and/or microbial composition of a subject, said method comprising the method of the first aspect and wherein, preferably, the subject has previously had a sample analysed for one or more biomarkers, or any combination of biomarkers herein disclosed.

In a third aspect, the present invention provides a method of determining a suitable course of treatment for a subject having a vaginal infection, cervical dysplasia, unsuccessful in vitro fertilization, miscarriage and/or pre-term birth, the method comprising predicting the inflammatory state, level of mucosal immune activation and/or microbial composition of a subject using the method of the first aspect, and wherein a suitable course of treatment is determined if the predicted inflammatory state, level of mucosal immune activation and/or microbial composition of the subject is indicative of vaginal infection, cervical dysplasia, unsuccessful in vitro fertilization, miscarriage and/or pre-term birth.

In a fourth aspect, the present invention provides a method of monitoring the response of a subject to a treatment, said subject having a vaginal infection, cervical dysplasia, unsuccessful in vitro fertilization, miscarriage and/or pre-term birth, the method comprising predicting the inflammatory state, level of mucosal immune activation of a subject and/or microbial composition using the method of the first aspect, and wherein a suitable course of treatment is determined if the predicted inflammatory state, level of mucosal immune activation and/or microbial composition of the subject is indicative of vaginal infection, cervical dysplasia, unsuccessful in vitro fertilization, miscarriage and/or pre-term birth.

In a fifth aspect, the present invention provides a method of diagnosing a vaginal infection, cervical dysplasia, unsuccessful in vitro fertilisation, miscarriage and/or pre-term birth in a subject, wherein the method of the first aspect is performed on a sample obtained from the subject.

In a sixth aspect, the present invention provides a method of treating a vaginal infection in a subject, said method comprising the steps of: (i) analysing a mucosal sample obtained from the subject to identify the presence and/or level of one or more biomarkers, or any combination of biomarkers, wherein the biomarkers are selected from a panel of biomarkers, wherein the panel of biomarkers comprises amino-aminohexyl-diaminomethylideneamino-pentanamide, aminonaphthalene, amino-sulfanylheptenyl-amino-oxooctadecanoic acid, arachidic acid (C20:0), arginine, asparagine, aspartic acid, butynoate, cadaverine, carboxysulfanyl-pentanedioic acid, Cer(d24:1/18:1), cerotic acid (C26:0), DG(36:3), dihydroxy-octadecadienoic acid, dimethoxyphenyl-hexadecylcarbamothioyl-propenamide, docosanoic acid (C22:0), docosanylsulfanylbutanoic acid, docosenoic acid (C22:1), eicosadienoic acid (C20:2), eicosanoic acid, ethyl-hexadecylcarbamothioylamino-oxobutanoate, galabiosylceramide (d18:1/16:0), galbeta-Cer(d40:1), glutamate, glutamine, glutamyl-leucine, glycyl-phenylalanine, heptadecylenic acid (C17:1), heptadecyl-hydroxyimidazole, hexadecenylsuccinic acid, histamine, HODE, hydroxycaproic acid, hydroxyglutaric acid, hydroxyvaleric acid, lactoylcysteine, leucine, leucyl-alanine, leucyl-glutamine, leucyl-glycyl-glycine, leucyl-serine, leucyl-threonine, lignoceric acid (C24:0), linoleic acid (C18:2), linolenic acid (C18:3), lysine, lyso-PE(16:1), lyso-PE(18:1), lyso-PE(18:2), lyso-PE(20:4), lyso-PE(O-16:0), lyso-PG(16:0), lyso-PS(18:0), maltotriose, methoxymethyl methyl-glucopyranosiduronate, methyl-hexadecylcarbamothioylamino-oxobutanoate, N2-acetyl-ornithine, N-acetylcadaverine, N-acetylputrescine, nervonic acid (C24:1), nonadecanoic acid (C19:0), nonadecenoic acid (C19:1), N-palmitoyl glutamine, N-palmitoyl methionine, N-palmitoyl-cysteine, octadecanoyloxamide, octadecanyloxy-propoxy-thiazolyl-propanamide, octadecenylamino-sulfanylpropanoic acid, octadecyl-oxy-octadecanoic acid (SAHSA), oleic acid (C18:1), OXO-octadecadienoic acid, oxoproline, oxosulfanyl-oxazolidinyl-octadecanamide, oxypurinol, PC(O-14:1/18:2), pentacosanoic acid (C25:0), PG(18:1/18:2), phenyl sulfoxide, phenylalanine, phenylethylamine, phenyllactic acid, phosphatidyl glycerol, phytenic acid (C20:1), PI(20:4/18:0), propyl-hexadecylcarbamothioylamino-oxobutanoate, PS(16:0/18:1), PS(18:0/18:1), putrescine, pyridinamine, pyrrolidinecarboxaldehyde, stearic acid (18:0), styrene, succinic acid, thiomalic acid, tolualdehyde, tricosanoic acid (C23:0), tyramine, vaccenyl carnitine, valyl-alanine, valyl-glutamine, valyl-phenylalanine, valyl-serine, vinylaniline, and ximenic acid (C26:1); (ii) forming a metabolic profile, wherein said metabolic profile is formed from data showing the presence and/or level of the biomarkers identified in step (i); (iii) using a predictive multivariate statistical model and/or a machine learning model pre-trained with metabolic and immuno-profiling datasets obtained from a reference population to predict the subject's mucosal inflammatory state and/or level of mucosal immune activation; and wherein the method further comprises the step of associating the metabolic profile of step ii) with metataxonomic data from a reference population to obtain the microbial composition of the mucosal sample, wherein when the subject's mucosal inflammatory state, level of mucosal immune activation and/or microbial composition are indicative of a vaginal infection, the subject is administered an anti-inflammatory agent, an antibiotic, a live biotherapeutic, a prebiotic, a probiotic, P4 or advised to undergo a cervical cerclage procedure.

In a seventh aspect, the present invention provides a method of preventing pre-term birth in a subject, said method comprising the steps of: (i) analysing a mucosal sample obtained from the subject to identify the presence and/or level of one or more biomarkers, or any combination of biomarkers, wherein the biomarkers are selected from a panel of biomarkers, wherein the panel of biomarkers comprises amino-aminohexyl-diaminomethylideneamino-pentanamide, aminonaphthalene, amino-sulfanylheptenyl-amino-oxooctadecanoic acid, arachidic acid (C20:0), arginine, asparagine, aspartic acid, butynoate, cadaverine, carboxysulfanyl-pentanedioic acid, Cer(d24:1/18:1), cerotic acid (C26:0), DG(36:3), dihydroxy-octadecadienoic acid, dimethoxyphenyl-hexadecylcarbamothioyl-propenamide, docosanoic acid (C22:0), docosanylsulfanylbutanoic acid, docosenoic acid (C22:1), eicosadienoic acid (C20:2), eicosanoic acid, ethyl-hexadecylcarbamothioylamino-oxobutanoate, galabiosylceramide (d18:1/16:0), galbeta-Cer(d40:1), glutamate, glutamine, glutamyl-leucine, glycyl-phenylalanine, heptadecylenic acid (C17:1), heptadecyl-hydroxyimidazole, hexadecenylsuccinic acid, histamine, HODE, hydroxycaproic acid, hydroxyglutaric acid, hydroxyvaleric acid, lactoylcysteine, leucine, leucyl-alanine, leucyl-glutamine, leucyl-glycyl-glycine, leucyl-serine, leucyl-threonine, lignoceric acid (C24:0), linoleic acid (C18:2), linolenic acid (C18:3), lysine, lyso-PE(16:1), lyso-PE(18:1), lyso-PE(18:2), lyso-PE(20:4), lyso-PE(O-16:0), lyso-PG(16:0), lyso-PS(18:0), maltotriose, methoxymethyl methyl-glucopyranosiduronate, methyl-hexadecylcarbamothioylamino-oxobutanoate, N2-acetyl-ornithine, N-acetylcadaverine, N-acetylputrescine, nervonic acid (C24:1), nonadecanoic acid (C19:0), nonadecenoic acid (C19:1), N-palmitoyl glutamine, N-palmitoyl methionine, N-palmitoyl-cysteine, octadecanoyloxamide, octadecanyloxy-propoxy-thiazolyl-propanamide, octadecenylamino-sulfanylpropanoic acid, octadecyl-oxy-octadecanoic acid (SAHSA), oleic acid (C18:1), oxo-octadecadienoic acid, oxoproline, oxosulfanyl-oxazolidinyl-octadecanamide, oxypurinol, PC(O-14:1/18:2), pentacosanoic acid (C25:0), PG(18:1/18:2), phenyl sulfoxide, phenylalanine, phenylethylamine, phenyllactic acid, phosphatidyl glycerol, phytenic acid (C20:1), PI(20:4/18:0), propyl-hexadecylcarbamothioylamino-oxobutanoate, PS(16:0/18:1), PS(18:0/18:1), putrescine, pyridinamine, pyrrolidinecarboxaldehyde, stearic acid (18:0), styrene, succinic acid, thiomalic acid, tolualdehyde, tricosanoic acid (C23:0), tyramine, vaccenyl carnitine, valyl-alanine, valyl-glutamine, valyl-phenylalanine, valyl-serine, vinylaniline, and ximenic acid (C26:1); (ii) forming a metabolic profile, wherein said metabolic profile is formed from data showing the presence and/or level of the biomarkers identified in step (i); (iii) using a predictive multivariate statistical model and/or a machine learning model pre-trained with metabolic and immuno-profiling datasets obtained from a reference population to predict the subject's mucosal inflammatory state and/or level of mucosal immune activation; and wherein the method further comprises the step of associating the metabolic profile of step ii) with metataxonomic data from a reference population to obtain the microbial composition of the mucosal sample, wherein when the subject's mucosal inflammatory state, level of mucosal immune activation and/or microbial composition are indicative of pre-term birth, the subject is administered a preventative therapy or undergoes a preventative procedure.

In an eighth aspect, the present invention provides a kit comprising a first device arranged and adapted to direct a spray of charged droplets onto a surface of a swab in order to generate a plurality of analyte ions; a second device arranged and adapted to analyse said analyte ions, wherein said analyte ions are analysed for the presence and/or level of one or more biomarkers, or any combination of biomarkers, wherein the biomarkers are selected from a panel of biomarkers, wherein the panel of biomarkers comprises amino-aminohexyl-diaminomethylideneamino-pentanamide, aminonaphthalene, amino-sulfanylheptenyl-amino-oxooctadecanoic acid, arachidic acid (C20:0), arginine, asparagine, aspartic acid, butynoate, cadaverine, carboxysulfanyl-pentanedioic acid, Cer(d24:1/18:1), cerotic acid (C26:0), DG(36:3), dihydroxy-octadecadienoic acid, dimethoxyphenyl-hexadecylcarbamothioyl-propenamide, docosanoic acid (C22:0), docosanylsulfanylbutanoic acid, docosenoic acid (C22:1), eicosadienoic acid (C20:2), eicosanoic acid, ethyl-hexadecylcarbamothioylamino-oxobutanoate, galabiosylceramide (d18:1/16:0), galbeta-Cer(d40:1), glutamate, glutamine, glutamyl-leucine, glycyl-phenylalanine, heptadecylenic acid (C17:1), heptadecyl-hydroxyimidazole, hexadecenylsuccinic acid, histamine, HODE, hydroxycaproic acid, hydroxyglutaric acid, hydroxyvaleric acid, lactoylcysteine, leucine, leucyl-alanine, leucyl-glutamine, leucyl-glycyl-glycine, leucyl-serine, leucyl-threonine, lignoceric acid (C24:0), linoleic acid (C18:2), linolenic acid (C18:3), lysine, lyso-PE(16:1), lyso-PE(18:1), lyso-PE(18:2), lyso-PE(20:4), lyso-PE(O-16:0), lyso-PG(16:0), lyso-PS(18:0), maltotriose, methoxymethyl methyl-glucopyranosiduronate, methyl-hexadecylcarbamothioylamino-oxobutanoate, N2-acetyl-ornithine, N-acetylcadaverine, N-acetylputrescine, nervonic acid (C24:1), nonadecanoic acid (C19:0), nonadecenoic acid (C19:1), N-palmitoyl glutamine, N-palmitoyl methionine, N-palmitoyl-cysteine, octadecanoyloxamide, octadecanyloxy-propoxy-thiazolyl-propanamide, octadecenylamino-sulfanylpropanoic acid, octadecyl-oxy-octadecanoic acid (SAHSA), oleic acid (C18:1), oxo-octadecadienoic acid, oxoproline, oxosulfanyl-oxazolidinyl-octadecanamide, oxypurinol, PC(O-14:1/18:2), pentacosanoic acid (C25:0), PG(18:1/18:2), phenyl sulfoxide, phenylalanine, phenylethylamine, phenyllactic acid, phosphatidyl glycerol, phytenic acid (C20:1), PI(20:4/18:0), propyl-hexadecylcarbamothioylamino-oxobutanoate, PS(16:0/18:1), PS(18:0/18:1), putrescine, pyridinamine, pyrrolidinecarboxaldehyde, stearic acid (18:0), styrene, succinic acid, thiomalic acid, tolualdehyde, tricosanoic acid (C23:0), tyramine, vaccenyl carnitine, valyl-alanine, valyl-glutamine, valyl-phenylalanine, valyl-serine, vinylaniline, and ximenic acid (C26:1).

The inventors of the present invention have surprisingly discovered that the method herein allows for the simultaneous measurement of both mucosal membrane microbes and immune responses within the same clinical sample, resulting in an efficient and streamlined process compared to the currently used diagnostic methods. Further, the use of specific mass-spectrometry (MS) approaches (for example, desorption electrospray ionisation-MS) to provide direct on-swab metabolic profiling enables the resulting chemical signatures, or chemical “fingerprints”, of the method herein disclosed to be obtained rapidly, for example, in less than 3 minutes, making the present invention particularly advantageous for point-of-care settings. The method herein disclosed, wherein MS metabolic signatures are combined with metataxonomic and immuno-profiling data, surprisingly results in chemical signatures that robustly reflect not only the mucosal microbiota composition, but also the local inflammatory status of the subject. This simultaneous characterisation of both the microbial composition and immune status allows for a more detailed subject profile and as such is key in providing efficient and effective treatment to a subject, informing future treatment plans, and monitoring the progress of a subject to allow for a flexible and personalised approach to current and future treatment.

### DESCRIPTION OF FIGURES

FIG. 1 shows a Table in which the demographics and clinical characteristics of the study cohorts are displayed.

FIG. 2 shows a schematic illustration detailing longitudinal multi-omic sample analysis workflow of cervicovaginal swab samples collected from pregnant women from two independent patient cohorts (VMET:n=165, 455 swabs; VMET2:n=205, 573 swabs). Three biological replicate samples were collected from patients at up to five different time-points throughout gestation; 8-16, 16-19, 19-26, 26-30, and 32-38 weeks. In the VMET cohort, matching samples were used for 16S rRNA amplicon-based metataxonomic profiling, bacterial cultivation and metabolite profiling. Swab samples dedicated for metabolite profiling was first analysed by DESI-MS followed by liquid extraction and LC-MS profiling for further validation and cross-platform comparison. Matching samples collected in the VMET2 cohort were used for 16S rRNA amplicon-based metataxonomic profiling, DESI-MS metabolite profiling and immune-profiling using multiplexed assays. Linear mixed modelling was used for data integration of multi-omics data to derive DESI-MS metabolic signatures characteristic of vaginal microbiota composition and immune status.

FIG. 3 shows hierarchical clustering analysis of the 16S rRNA sequencing data from the VMET and VMET2 studies, and auxiliary diagnostic plots. A, B) Heatmap of log-transformed counts per microbial taxa found in the vaginal bacterial communities from the two patient cohorts. Hierarchical clustering of samples was based on their raw OTU counts aggregated (by summing) at species level, using Ward's method and the square-root of the Jensen-Shannon divergence as distance metric. C,D) Analysis of the average silhouette scores suggests an optimal (maximum average silhouette score) number of 11 and 9 clusters, for the VMET and VMET2 datasets, respectively. To facilitate comparison of results between the cohorts, clusters were coalesced into a smaller set of clusters (6 for VMET2 and 5 for VMET) consistent with previously described community state types: I=L. crispatus dominated; II=L. gasseri dominated; III=L. iners dominated; IV=mixed Gardnerella vaginalis+anaerobic bacteria; V=L. jensenii dominated; VI=Bifidobacterium breve dominated (VMET2 only); Sample specific silhouette score values representing how each sample fits into the final cluster in E) VMET and F) VMET2 (F). In both studies, silhouette scores were lowest for samples assigned to the CST IV cluster.

FIG. 4 shows heatmaps representing relative concentrations of DESI-MS (negative mode) derived metabolic features (n=88) significantly differing between Lactobacillus spp. dominated and Lactobacillus spp. depleted states in both independent patient cohorts. (CST I, L.crispatus; CST II, L. gasseri; CST III, L.iners; CST IV, mixed anaerobes; CST V, L. jensenii; CST VI, Bifidobacterium; CST VII Lactobacillus other).

FIG. 5 shows a Table wherein distribution of community state types (CST I, L.crispatus; CST II, L. gasseri; CST III, Liners; CST IV, mixed anaerobes; CST V, L. jensenii; CST VI, Bifidobacterium; CST VII Lactobacillus other) according to ethnicity (Caucasian, Asian, Black, Others) and gestation at birth (<28, 28-34, 34-37, >37 weeks) in the VMET and VMET2 cohorts.

FIG. 6 shows a Table displaying metabolites identified by DESI-MS to discriminate between Lactobacillus spp.—dominated and Lactobacillus spp.—depleted vaginal microbiomes data. Metabolic features were identified using both negative and positive ion polarity modes in both VMET and VMET2 patient cohorts with ppm mass error, putative annotation, MS/MS and sub class of metabolic compounds reported. Statistical significance was assessed via linear mixed effect model (LME) and Benjamini-Hochberg (BH) correction (q<0.05) with additional reported p-value and t-ratio. *Measured m/z values highlighted in bold were additionally corrected by replacing value found in the sample with the highest averaged measured m/z value in the patient cohort.

FIG. 7 shows boxplots of representative discriminatory metabolic features identified thiomalic acid, leucyl-serine, docosanoic acid (C22:0), lignoceric acid (C24:0) with calculated z-score measured in the two patient cohorts VMET2 (left) and VMET (right).

FIG. 8 shows the comparison of classification performance between the contrast LDOM vs LDEPL, I vs IV; I vs III; III vs IV in VMET2 and VMET using ROC curve analysis of DESI-MS data.

FIG. 9 shows ROC curve analysis plots and calculated AUC values for the random forest classifiers trained to distinguish Lactobacillus spp.—dominated (LDOM) versus Lactobacillus spp.—depleted (LDEPL) (column 1) vaginal microbiome compositions using metabolic profiles obtained with different MS based assays (RP-LC-MS Metabolite (−) (row 1), RP-LC-MS Lipid (−) (row 2), HILIC LC-MS (−) (row 3), RP-LC-MS Metabolite (+) (row 4), RP-LC-MS Lipid (+) (row 5)). Similar analyses were performed between major CST groups; CST I versus CST IV (column 2), CST I versus CST III (column 3), and CST III versus CST IV (column 4).

FIG. 10 shows a Table demonstrating the classification performance measures for assessing the VMC at genus and species levels using metabolomics profiling data obtained from the VMET and VMET2 cohort. A random forest classifier was used to discriminate between two classes for each contrast (LDOM vs LDEPL, I vs IV, I vs III and III vs IV), using as input the metabolomics data obtained from different MS platforms and modes (DESI-MS +/−, RP-LC-MS metabolites +/−, RP-LC-MS lipids +/−, HILIC-LC-MS−). A) Overview of sample and metabolite feature number used per mode for the classification performance analysis. B) Summary of predictive performance measures (Area under the curve (AUC), prAUC, accuracy, kappa, F1, sensitivity, specificity, positive predictive value (PPV), negative predictive value (NPV), prevalence, precision, recall, detection rate and balanced accuracy) for each tested contrast and mode. All values shown were estimated from the cross-validation test-set samples, using repeated (15 repeats) 5-fold cross-validation.

FIG. 11 shows metabolite platform comparison of misclassified samples found during the LDOM versus LDEPL comparison in the VMET cohort. Venn diagram representing numbers of matching samples with same misclassified LDOM vs LDEPL status using different metabolic profiling assays (DESI-MS, HILIC-LC-MS, RP-LC-MS lipids, RP-LC-MS metabolites) using data obtained in both negative (A) and positive (B) ion mode.

FIG. 12 shows assessment of host response at the mucosal interface using direct on-swab DESI-MS profiling, displaying cross-validated R²-value for all 22 corresponding measured immune mediators concentrations.

FIG. 13 shows a Table displaying statistically significant metabolite features found associated with host-related immune responses in cervicovaginal swab samples using DESI-MS data acquired in negative ion mode in the VMET2 patient cohort with reported ppm mass error, putative annotation, MS/MS and sub class of metabolic compounds. *Measured m/z values highlighted in bold were additionally corrected by replacing value found in the sample with the highest averaged measured m/z value in the patient cohort.

FIG. 14 shows the association between predicted log-transformed value of immune marker by DESI-MS and measured log-transformed values by multiplexed immune-assay for IL-1β (CV R²=0.51), IL-8 (CV R²=0.37), C3b/iC3b (CV R²=0.33), IgG3 (R²=0.31), IgG2 (CV R²=0.27), MBL (CV R²=0.26). A linear regression line was fitted to the log-transformed values and their corresponding prediction.

FIG. 15 shows the prediction of log (Immune Marker) using top 15 metabolites identified by DESI-MS. Cross-validated R2 are reported as mean R2 from CV +−1 standard deviation. Results were obtained with random forest regressor, repeated (n=15, 7-fold cross-validation).

FIG. 16 shows cross-validation metrics and model performance for top 15 metabolites used to predict microbial composition in two different patient populations (VMET1 n=160 patient, 455 samples and VMET 2 n=205, 573 samples).

FIG. 17 shows ROC curve for the prediction of Lactobacillus dominated (optimal) versus Lactobacillus depleted/high diversity (sub-optimal) vaginal bacteria composition using the top 15 metabolites described in claim 1. Results were obtained using a random forest classifier and n=15 stratified 7-fold cross-validation.

FIG. 18 shows increased risk of PTB was associated with vaginal microbiome instability (defined by shifts between Lactobacillus spp. dominated (LDOM) and Lactobacillus spp. depleted (LDEPL) compositions) measured by 16S rRNA-based metataxonomics (OR 1.97, CI 1.03-1.66, p=0.04) or predicted using DESI-MS profiles (OR 1.47, CI:0.75-2.78, p=0.25).

FIG. 19 shows LDEPL vaginal composition associated with increased IL-1β levels compared to LDOM, however highest levels were observed in LDEPL women subsequently having preterm delivery. This relationship was also observed when IL-1β levels and vaginal microbiota composition were predicted using direct swab profiling by DESI-MS.

FIG. 20 shows a relationship between LDEPL, increased MBL and subsequent preterm birth was also detected by DESI-MS profiling.

FIG. 21 shows elevated MBL in response to cervical cerclage performed with braided suture material, but not monofilament.

FIG. 22 shows elevated IL-1β levels in response to cervical cerclage performed with braided suture material, but not monofilament.

FIG. 23 shows preterm birth in women treated with cervical cerclage using braided suture material was associated with higher IL-1β levels compared to term birth outcomes, whereas no relationship between IL-1β levels measured or DESI-MS-predicted were observed with pregnancy outcome following cervical cerclage using monofilament suture.

### DETAILED DESCRIPTION

The following description is presented to enable any person skilled in the art to make and use the invention. Various modifications to the disclosed embodiments will be readily apparent to those skilled in the art.

In order that the present invention may be more readily understood, certain terms are first defined. Additional definitions are set forth throughout the detailed description.

As used herein, the term “inflammatory state” refers to the degree of inflammation occurring in a subject at a particular time.

As used herein, the term “immune activation” and “immune response” are used interchangeably and refers to the action of, for example, lymphocytes, antigen presenting cells, phagocytic cells, granulocytes, and soluble macromolecules produced by the above cells or the liver (including immunoglobulins, cytokines, chemokines, and complement molecules).

As used herein, the term “mucosal membrane”, “mucosa” and “mucosal surface” are used interchangeably and refers to any surface in the body that produces mucosal fluid. The mucosal membrane may be considered to be a protective layer responsible for trapping pathogens in the human body. The mucosa lines several passages and cavities of the body, particularly those with openings exposed to the external environment. Examples of locations of mucosal membranes include, but are not limited to, the digestive tract, the respiratory tracts and the reproductive tracts (both male and female). The mucosa comprises a mucus layer (the inner mucus layer); an epithelium; a basement membrane, a Lamina propria (LP), which is a layer of connective tissue; and a Muscularis mucosae, which is a thin layer of smooth muscle. Thus, the terms “mucosal membrane”, “mucosa”, “mucosal surface” used herein refer to this entire complex, unless stated otherwise. The mucosa may also be covered by a further, outer mucus layer, which is typically more loosely associated therewith. Any reference herein to a “mucosa” may include reference to this further, outer mucus layer.

As used herein, the terms “patient” and “subject” are used interchangeably and refer to any animal (e.g. mammal), including, but not limited to, humans, non-human primates, canines, felines, rodents and the like, which is to be where the sample is obtained from. Preferably, the subject or patient is a human.

As used herein, the term “simultaneous” in the context of the present invention refers to the ability of the method herein disclosed to predict both the microbial composition and immune response/inflammatory status of a subject using the same clinical sample.

As used herein, the term “biomarker” refers to a measurable indicator of a biological state or condition, for example, the inflammatory status of a subject. Biomarkers may be molecular or cellular in nature and can serve predictive, diagnostic and prognostic purposes.

As used herein, the term “metabolic profile”, “metabolic fingerprint”, “chemical signature” and “chemical fingerprint” are used interchangeably and refers to the measurement of numerous metabolites and their intermediates to provide a snapshot of the subject's microbial composition/inflammatory status at a particular point in time, for example, prior to and/or after a treatment.

As used herein, the term “reference population” refers to a group of well-defined and clinically phenotyped subjects whose samples are assayed to provide reference ranges for chemical signatures that inform the algorithms and models used to predict microbial composition and inflammatory state. Data collected from subsequently sampled individuals can then be compared and matched to this reference data enabling the microbial composition and inflammatory state to be predicted.

As used herein, the term “microbial composition” refers to the identification and/or determination of the level of different microbes within a given sample.

As used herein, the term “metabolic profiling technique” refers to any technique that is able to measure numerous metabolites and their intermediates to provide a snapshot of the subject's microbial composition/inflammatory status at a particular point in time. Preferably, DESI-MS is selected as the chosen metabolic profiling technique, however other techniques are by no means excluded.

The use of the alternative (e.g., “or”) should be understood to mean either one, both, or any combination thereof of the alternatives. As used herein, the indefinite articles “a” or “an” should be understood to refer to “one or more” of any recited or enumerated component.

As described above, the present invention is a method by which simultaneous measurements of both the microbial composition of a mucosal sample and the corresponding immune responses/inflammatory status of a subject can be obtained using the same clinical sample. This has numerous advantages, namely, a more efficient, rapid, cost-effective diagnostic assay that can be used in point-of-care settings, not only for diagnostic purposes, but for informing, and monitoring treatment strategies, personalised to the subject.

Accordingly, in a first aspect, the present invention provides a method comprising the steps of: (i) analysing a mucosal sample obtained from a subject to identify the presence and/or level of one or more biomarkers, or any combination of the biomarkers, wherein the biomarkers are selected from a panel of biomarkers, wherein the panel of biomarkers comprises amino-aminohexyl-diaminomethylideneamino-pentanamide, aminonaphthalene, amino-sulfanylheptenyl-amino-oxooctadecanoic acid, arachidic acid (C20:0), arginine, asparagine, aspartic acid, butynoate, cadaverine, carboxysulfanyl-pentanedioic acid, Cer(d24:1/18:1), cerotic acid (C26:0), DG(36:3), dihydroxy-octadecadienoic acid, dimethoxyphenyl-hexadecylcarbamothioyl-propenamide, docosanoic acid (C22:0), docosanylsulfanylbutanoic acid, docosenoic acid (C22:1), eicosadienoic acid (C20:2), eicosanoic acid, ethyl-hexadecylcarbamothioylamino-oxobutanoate, galabiosylceramide (d18:1/16:0), galbeta-Cer(d40:1), glutamate, glutamine, glutamyl-leucine, glycyl-phenylalanine, heptadecylenic acid (C17:1), heptadecyl-hydroxyimidazole, hexadecenylsuccinic acid, histamine, HODE, hydroxycaproic acid, hydroxyglutaric acid, hydroxyvaleric acid, lactoylcysteine, leucine, leucyl-alanine, leucyl-glutamine, leucyl-glycyl-glycine, leucyl-serine, leucyl-threonine, lignoceric acid (C24:0), linoleic acid (C18:2), linolenic acid (C18:3), lysine, lyso-PE(16:1), lyso-PE(18:1), lyso-PE(18:2), lyso-PE(20:4), lyso-PE(O-16:0), lyso-PG(16:0), lyso-PS(18:0), maltotriose, methoxymethyl methyl-glucopyranosiduronate, methyl-hexadecylcarbamothioylamino-oxobutanoate, N2-acetyl-ornithine, N-acetylcadaverine, N-acetylputrescine, nervonic acid (C24:1), nonadecanoic acid (C19:0), nonadecenoic acid (C19:1), N-palmitoyl glutamine, N-palmitoyl methionine, N-palmitoyl-cysteine, octadecanoyloxamide, octadecanyloxy-propoxy-thiazolyl-propanamide, octadecenylamino-sulfanylpropanoic acid, octadecyl-oxy-octadecanoic acid (SAHSA), oleic acid (C18:1), oxo-octadecadienoic acid, oxoproline, oxosulfanyl-oxazolidinyl-octadecanamide, oxypurinol, PC(O-14:1/18:2), pentacosanoic acid (C25:0), PG(18:1/18:2), phenyl sulfoxide, phenylalanine, phenylethylamine, phenyllactic acid, phosphatidyl glycerol, phytenic acid (C20:1), PI(20:4/18:0), propyl-hexadecylcarbamothioylamino-oxobutanoate, PS(16:0/18:1), PS(18:0/18:1), putrescine, pyridinamine, pyrrolidinecarboxaldehyde, stearic acid (18:0), styrene, succinic acid, thiomalic acid, tolualdehyde, tricosanoic acid (C23:0), tyramine, vaccenyl carnitine, valyl-alanine, valyl-glutamine, valyl-phenylalanine, valyl-serine, vinylaniline, and ximenic acid (C26:1); (ii) forming a metabolic profile, wherein said metabolic profile is formed from data showing the presence and/or level of the biomarkers identified in step (i); (iii) using a predictive multivariate statistical model and/or a machine learning model pre-trained with metabolic and immuno-profiling datasets obtained from a reference population to predict the subject's mucosal inflammatory state at a mucosal membrane and/or level of immune activation at a mucosal membrane; and wherein the method further comprises the step of associating the metabolic profile of step ii) with metataxonomic data from a reference population to obtain the microbial composition of the mucosal sample.

It is known that the activation of immune responses often accompanies a variety of disease states. For example, it is known that the activation of innate immune responses in the lower reproductive tract often accompanies disease states associated with suboptimal microbiota composition, including, but not limited to, bacterial vaginosis (BV), preterm birth, and sexually transmitted infections⁽¹⁻³⁾. However, current diagnosis of inflammation, or immune status of a subject, requires collection of additional samples and the undertaking of additional expensive and time-consuming tests and assays. In contrast, the method herein disclosed allows for information on both the microbial composition and the host's immune response/status to be obtained directly from mucosal swabs, without the requirement of sample preparation and within a fraction of the time.

To obtain the metabolic profile of a subject, the presence and/or level of various biomarkers are measured in a mucosal sample obtained from a subject. The skilled person will readily understand that a large number of combinations of biomarkers are possible from the panel disclosed in step (i) of the method. For example, the panel of biomarkers may comprise at least one biomarker, at least two biomarkers, at least three biomarkers, at least four biomarkers, at least five biomarkers, at least six biomarkers, at least seven biomarkers, at least eight biomarkers, at least nine biomarkers, at least ten biomarkers, at least eleven biomarkers, at least twelve biomarkers, at least thirteen biomarkers, at least fourteen biomarkers, at least fifteen biomarkers selected from the panel of biomarkers of step (i) of the method of the invention.

In one embodiment, the subject's mucosal inflammatory state and/or level of mucosal immune activation may be predicted using biomarkers selected from a panel of biomarkers comprising amino-sulfanylheptenyl-amino-oxooctadecanoic acid, Cer(d24:1/18:1), dimethoxyphenyl-hexadecylcarbamothioyl-propenamide, docosanoic acid (C22:0), docosenoic acid (C22:1), ethyl-hexadecylcarbamothioylamino-oxobutanoate, galabiosylceramide (d18:1/16:0), galbeta-Cer(d40:1), heptadecyl-hydroxyimidazole, hexadecenylsuccinic acid, lactoylcysteine, methyl-hexadecylcarbamothioylamino-oxobutanoate, N-palmitoyl glutamine, octadecanoyloxamide, oxosulfanyl-oxazolidinyl-octadecanamide, phytenic acid (C20:1), and PI(20:4/18:0), or any combination thereof.

The panel of biomarkers may further comprise any one or more biomarker selected from amino-aminohexyl-diaminomethylideneamino-pentanamide, aminonaphthalene, arachidic acid (C20:0), arginine, asparagine, aspartic acid, butynoate, cadaverine, carboxysulfanyl-pentanedioic acid, cerotic acid (C26:0), DG(36:3), dihydroxy-octadecadienoic acid, docosanylsulfanylbutanoic acid, eicosadienoic acid (C20:2), eicosanoic acid, glutamate, glutamine, glutamyl-leucine, glycyl-phenylalanine, heptadecylenic acid (C17:1), histamine, HODE, hydroxycaproic acid, hydroxyglutaric acid, hydroxyvaleric acid, leucine, leucyl-alanine, leucyl-glutamine, leucyl-glycyl-glycine, leucyl-serine, leucyl-threonine, lignoceric acid (C24:0), linoleic acid (C18:2), linolenic acid (C18:3), lysine, lyso-PE (16:1), lyso-PE(18:1), lyso-PE(18:2), lyso-PE(20:4), lyso-PE(O-16:0), lyso-PG(16:0), lyso-PS(18:0), maltotriose, methoxymethyl methyl-glucopyranosiduronate, N2-acetyl-ornithine, N-acetylcadaverine, N-acetylputrescine, nervonic acid (C24:1), nonadecanoic acid (C19:0), nonadecenoic acid (C19:1),, N-palmitoyl methionine, N-palmitoyl-cysteine, octadecanyloxy-propoxy-thiazolyl-propanamide, octadecenylamino-sulfanylpropanoic acid, octadecyl-oxy-octadecanoic acid (SAHSA), oleic acid (C18:1), oxo-octadecadienoic acid, oxoproline, oxypurinol, PC(O-14:1/18:2), pentacosanoic acid (C25:0), PG(18:1/18:2), phenyl sulfoxide, phenylalanine, phenylethylamine, phenyllactic acid, phosphatidyl glycerol, propyl-hexadecylcarbamothioylamino-oxobutanoate, PS(16:0/18:1), PS(18:0/18:1), putrescine, pyridinamine, pyrrolidinecarboxaldehyde, stearic acid (18:0), styrene, succinic acid, thiomalic acid, tolualdehyde, tricosanoic acid (C23:0), tyramine, vaccenyl carnitine, valyl-alanine, valyl-glutamine, valyl-phenylalanine, valyl-serine, vinylaniline, and ximenic acid (C26:1), or any combination thereof.

In one embodiment, the microbial composition of the mucosal sample may be predicted using biomarkers selected from a panel of biomarkers comprising amino-sulfanylheptenyl-amino-oxooctadecanoic acid, dimethoxyphenyl-hexadecylcarbamothioyl-propenamide, docosanoic acid (C22:0), docosenoic acid (C22:1), eicosadienoic acid (C20:2), ethyl-hexadecylcarbamothioylamino-oxobutanoate, hydroxycaproic acid, hydroxyvaleric acid, leucyl-serine, lignoceric acid (C24:0), nonadecanoic acid (C19:0), nonadecenoic acid (C19:1), N-palmitoyl-cysteine, oxosulfanyl-oxazolidinyl-octadecanamide, phytenic acid (C20:1), thiomalic acid, and tricosanoic acid (C23:0), or any combination thereof.

The panel of biomarkers may further comprise amino-aminohexyl-diaminomethylideneamino-pentanamide, aminonaphthalene, arachidic acid (C20:0), arginine, asparagine, aspartic acid, butynoate, cadaverine, carboxysulfanyl-pentanedioic acid, Cer(d24:1/18:1), cerotic acid (C26:0), DG(36:3), dihydroxy-octadecadienoic acid, docosanylsulfanylbutanoic acid, eicosanoic acid, galabiosylceramide (d18:1/16:0), galbeta-Cer(d40:1), glutamate, glutamine, glutamyl-leucine, glycyl-phenylalanine, heptadecylenic acid (C17:1), heptadecyl-hydroxyimidazole, hexadecenylsuccinic acid, histamine, HODE, hydroxyglutaric acid, lactoylcysteine, leucine, leucyl-alanine, leucyl-glutamine, leucyl-glycyl-glycine, leucyl-threonine, linoleic acid (C18:2), linolenic acid (C18:3), lysine, lyso-PE(16:1), lyso-PE(18:1), lyso-PE(18:2), lyso-PE(20:4), lyso-PE(O-16:0), lyso-PG(16:0), lyso-PS(18:0), maltotriose, methoxymethyl methyl-glucopyranosiduronate, methyl-hexadecylcarbamothioylamino-oxobutanoate, N2-acetyl-ornithine, N-acetylcadaverine, N-acetylputrescine, nervonic acid (C24:1), N-palmitoyl glutamine, N-palmitoyl methionine, octadecanoyloxamide, octadecanyloxy-propoxy-thiazolyl-propanamide, octadecenylamino-sulfanylpropanoic acid, octadecyl-oxy-octadecanoic acid (SAHSA), oleic acid (C18:1), oxo-octadecadienoic acid, oxoproline,, oxypurinol, PC(O-14:1/18:2), pentacosanoic acid (C25:0), PG(18:1/18:2), phenyl sulfoxide, phenylalanine, phenylethylamine, phenyllactic acid, phosphatidyl glycerol, PI(20:4/18:0), propyl-hexadecylcarbamothioylamino-oxobutanoate, PS(16:0/18:1), PS(18:0/18:1), putrescine, pyridinamine, pyrrolidinecarboxaldehyde, stearic acid (18:0), styrene, succinic acid, tolualdehyde, tyramine, vaccenyl carnitine, valyl-alanine, valyl-glutamine, valyl-phenylalanine, valyl-serine, vinylaniline, and ximenic acid (C26:1), or any combination thereof.

In another embodiment, the subject's mucosal inflammatory state, level of mucosal immune activation and/or microbial composition of the mucosal sample may be predicted using biomarkers selected from a panel of biomarkers comprising amino-sulfanylheptenyl-amino-oxooctadecanoic acid, Cer(d24:1/18:1), dimethoxyphenyl-hexadecylcarbamothioyl-propenamide, docosanoic acid (C22:0), docosenoic acid (C22:1), ethyl-hexadecylcarbamothioylamino-oxobutanoate, galabiosylceramide (d18:1/16:0), galbeta-Cer(d40:1), heptadecyl-hydroxyimidazole, hexadecenylsuccinic acid, lactoylcysteine, methyl-hexadecylcarbamothioylamino-oxobutanoate, N-palmitoyl glutamine, octadecanoyloxamide, oxosulfanyl-oxazolidinyl-octadecanamide, phytenic acid (C20:1), PI(20:4/18:0), eicosadienoic acid (C20:2), hydroxycaproic acid, hydroxyvaleric acid, leucyl-serine, lignoceric acid (C24:0), nonadecanoic acid (C19:0), nonadecenoic acid (C19:1), N-palmitoyl-cysteine, thiomalic acid, and tricosanoic acid (C23:0), or any combination thereof. Alternatively, the biomarkers may be selected from the panel comprising amino-sulfanylheptenyl-amino-oxooctadecanoic acid, dimethoxyphenyl-hexadecylcarbamothioyl-propenamide, docosanoic acid (C22:0), docosenoic acid (C22:1), ethyl-hexadecylcarbamothioylamino-oxobutanoate, oxosulfanyl-oxazolidinyl-octadecanamide, phytenic acid (C20:1), or any combination thereof. In such an embodiments, the panel of biomarkers may further comprise amino-aminohexyl-diaminomethylideneamino-pentanamide, aminonaphthalene, arachidic acid (C20:0), arginine, asparagine, aspartic acid, butynoate, cadaverine, carboxysulfanyl-pentanedioic acid, cerotic acid (C26:0), DG(36:3), dihydroxy-octadecadienoic acid, docosanylsulfanylbutanoic acid, eicosanoic acid, glutamate, glutamine, glutamyl-leucine, glycyl-phenylalanine, heptadecylenic acid (C17:1), histamine, HODE, hydroxyglutaric acid, leucine, leucyl-alanine, leucyl-glutamine, leucyl-glycyl-glycine, leucyl-threonine, linoleic acid (C18:2), linolenic acid (C18:3), lysine, lyso-PE(16:1), lyso-PE(18:1), lyso-PE(18:2), lyso-PE(20:4), lyso-PE(O-16:0), lyso-PG(16:0), lyso-PS(18:0), maltotriose, methoxymethyl methyl-glucopyranosiduronate, N2-acetyl-ornithine, N-acetylcadaverine, N-acetylputrescine, nervonic acid (C24:1), N-palmitoyl methionine, octadecanyloxy-propoxy-thiazolyl-propanamide, octadecenylamino-sulfanylpropanoic acid, octadecyl-oxy-octadecanoic acid (SAHSA), oleic acid (C18:1), oxo-octadecadienoic acid, oxoproline, oxypurinol, PC(O-14:1/18:2), pentacosanoic acid (C25:0), PG(18:1/18:2), phenyl sulfoxide, phenylalanine, phenylethylamine, phenyllactic acid, phosphatidyl glycerol, propyl-hexadecylcarbamothioylamino-oxobutanoate, PS(16:0/18:1), PS(18:0/18:1), putrescine, pyridinamine, pyrrolidinecarboxaldehyde, stearic acid (18:0), styrene, succinic acid, tolualdehyde, tyramine, vaccenyl carnitine, valyl-alanine, valyl-glutamine, valyl-phenylalanine, valyl-serine, vinylaniline, and ximenic acid (C26:1), or any combination thereof.

The prediction of the subject's inflammatory state, level of mucosal immune activation and/or microbial composition may be based solely on the metabolic profile acquired from the mucosal membrane sample, which is subsequently used in a predictive multivariate statistical model and/or a machine learning model pre-trained with metabolic and immuno-profiling datasets obtained from a reference population to predict the subject's mucosal inflammatory state level of mucosal immune activation. Accordingly, the present invention has the advantage that no further information in addition to that obtained in step (i) of the method is required to obtain a robust and accurate reflection of the subject's mucosal inflammatory state and/or level of mucosal immune activation. However, it is understood that in certain situations additional assays or tests may be run to support or further investigate the findings of the method herein disclosed.

The subject's mucosal inflammatory state, level of mucosal immune activation and/or microbial composition may be obtained simultaneously. As used herein, the term “simultaneously” refers to the mucosal inflammatory state, level of mucosal immune activation and the microbial composition of the mucosal sample being obtained at substantially the same time using the same clinical sample.

The method herein uses predictive multivariate statistical models and/or machine learning models pre-trained with metabolic and immuno-profiling datasets obtained from a reference population to predict the subject's mucosal inflammatory state and/or level of mucosal immune activation in an accurate and efficient way. In one embodiment, the immuno-profiling dataset obtained from the reference population may be formed from data showing the presence and/or level of one or more biomarkers, wherein the biomarkers are selected from the group comprising cytokines, chemokines, immunoglobulins, growth factors, complement component molecules and/or any other cellular immune markers. For example, the immuno-profiling dataset obtained from the reference population may be formed from data showing the presence and/or level of one or more biomarkers selected from IFN-γ, IL-1α, IL-1β, IL-1RA, IL-2, IL-3, IL-4, IL-5, IL-6, IL-7, IL-8, IL-9, IL-10, IL-11, IL-12, IL-13, IL-14, IL-15, IL-16, IL-17, IL-18, IL-19, IL-20, IL-21, IL-22, IL-23, IL-24, IL-25, IL-26, IL-27, IL-28, IL-29, IL-30, IL-31, IL-32, IL-33, IL-34, IL-35, IL-36, CCL2, CCL3, CCL4, CCL5, CCL6, CCL7, CCL8, CCL9, CCL10, CCL11, CCL12, CCL13, CCL14, CCL15, CCL16, CCL17, CCL18, CCL19, CCL20,

CCL21, CCL22, CCL23, CCL24, CCL25, CCL26, CCL27, CCL28, CXCL1, CXCL2, CXCL3, CXCL4, CXCL5, CXCL6, CXCL7, CXCL8, CXCL9, CXCL10, CXCL11, CXCL12, CXCL13, CXCL14, CXCL15, CXCL16, CXCL17, XCL1, XCL2, CX3CL1, CCR1, CCR2, CCR3, CCR4, CCR5, CCR6, CCR7, CCR8, CCR9, CCR10, CXCR1, CXCR2, CXCR3, CXCR3B, CXCR4, CXCR5, CXCR6, CXCR7, XCR1, CX3CR1, IgG, IgG1, IgG2, IgG3, IgG4, IgM, IgA, IgD, IgE, CNTF, LIF, MCSF, GCSF, GMCSF, EGF, FGF1-23, GDNF, HGF, HDGF, IGF1, IGF2, KGF, MSF, MSP, NRG1-4, BDNF, NGF, NT3, NT4, PDGF, TGFα, TGFβ, TNFα, VEGF, C1, C2, C3, C3a, C3b, C4, C5, C5a, C5b, C6, C7, C8, C9, Factor B, MBL, MASP-1, and/or MASP-2 or any combinations thereof. However, it is also understood that any other immune marker may also be used in the creation of a reference population dataset.

In one preferred embodiment, the immuno-profiling dataset obtained from the reference population may be formed from data show showing the presence and/or level of one or more biomarkers selected from IL-1β, IL-2, IL-4, IL-5, IL-6, IL-8, IL-10, IL-18, IFN-γ, GM-CSF, TNF-α, IgA, IgG1, IgG2, IgG3, IgG4, IgM, C3b, C5, C5a, and/or MBL, or any combinations thereof.

The method herein disclosed provides a snapshot of what the microbial composition/inflammatory status of a mucosal sample is at a single point in time. It is envisaged that this is particularly advantageous to aid in the diagnosis and monitoring of diseases/disorders associated with the reproductive tract, for example, BV, HIV, sexually transmitted diseases, miscarriage, pre-term birth and unsuccessful in vitro fertilisation. However, it will be readily apparent to the skilled person that the method herein disclosed has numerous applications associated with other mucosal membranes in the body, and is not limited to the mucosal membrane being, for example, a vaginal mucosa membrane.

Accordingly, the mucosal membrane may be a bronchial mucosa membrane, a uterine mucosa membrane, an oesophageal mucosa membrane, a gastric mucosa membrane, an intestinal mucosa membrane, a nasal mucosa membrane, an olfactory mucosa membrane, an oral mucosa membrane, a penile mucosa membrane or a vaginal mucosa membrane. As such, the method herein disclosed may also be particularly useful for the diagnosis and/or monitoring of diseases/disorders associated with the gut, the respiratory system, ear/nasal/throat disorders, the male reproductive system, or any other system in which determining the microbial composition/inflammatory state would be beneficial. In a preferred embodiment, the mucosal membrane is a vaginal mucosa membrane.

The mucosal sample may be collected using any suitable device or sampling method that allows the sampling of mucosal surfaces. Such devices/sampling methods include, but are not limited to, brushes (for example, cervical sampling brush or a Tao brush), swabs and filter papers. In a preferred embodiment, the mucosal sample may be a swab sample. The swab may be a medical swab, a disposable swab, a cotton swab, a rayon swab, a plastic swab or a foam swab. The swab may be arranged and adapted for the mucosal membranes disclosed herein. Preferably, the swab has been modified to enhance selectivity for any one of the selected biomarkers, for example, the swab may be chemically modified to render the swab lipophilic, or the swab may involve the formation of a coating on the surface of the swab. Said coating may be a polymeric coating, for example, polydivinylbenzene (DVB), a copolymer of Nvinylpyrrolidone and divinylbenzene, or polydimethylsiloxane. The swab sample may be collected by contacting the swab with a mucosal surface in vivo, ex vivo or in vitro. In a preferred embodiment, the swab sample is a cervicovaginal swab sample or a nasal swab sample. In a most preferred embodiment, the swab sample is a cervicovaginal swab sample.

The present invention provides a method of direct on-swab metabolic profiling to rapidly characterize the metabolome of the selected mucosal membrane, for example, the cervicovaginal metabolome. As such, the mucosal sample may be analysed using mass spectrometry or any other metabolic profiling technique. The mucosal sample may be analysed using an ambient spectroscopy technique. A number of different ambient ionisation techniques are known and are intended to fall within the scope of the present invention. Desorption Electrospray Ionisation (“DESI”) was the first ambient ionisation technique to be developed and was disclosed in 2004. Since 2004, a number of other ambient ionisation techniques have been developed. These ambient ionisation techniques differ in their precise ionisation method but they share the same general capability of generating gas-phase ions directly from native (i.e. untreated or unmodified) samples. A particular benefit of the various ambient ionisation techniques which fall within the scope of the present invention is that the various ambient ionization techniques do not require any prior sample preparation. As a result, the various ambient ionisation techniques enable both in vivo and ex vivo tissue samples to be analysed without necessitating the time and expense of adding a matrix or reagent to the tissue sample or other target material. Examples of ambient spectroscopy techniques include, but are not limited to, DESI, DeSSI, DAPPI, EASI, JeDI, TM-DESI, LMJ-SSP, DICE, Nano-DESI, EADESI, APTDCI, V-EASI, AFAI, LESA, PTC-ESI, AFADESI, DEFFI, ESTASI, PASIT, DAPCI, DART, ASAP, APTDI, PADI, DBDI, FAPA, HAPGDI, APGDDI, LTP, LS-APGD, MIPDI, MFGDP, RoPPI, PLASI, MALDESI, ELDI, LDTD, LAESI, CALDI, LA-FAPA, LADESI, LDESI, LEMS, LSI, IR-LAMICI, LDSPI, PAMLDI, HALDI, PALDI, ESSI, PESI, ND-ESSI, PS, DI-APCI, TD, Wooden-tip, CBS-SPME, TSI, RADIO, LIAD-ESI, SAWN, UASI, SPA-nanoESI, PAUSI, DPESI, ESA-Py, APPIS, RASTIR, SACI, DEMI, REIMS, SPAM, TDAMS, MAN, SAII, SwiFERR and LPTD. Preferably, the ambient spectroscopy technique is desorption electrospray ionisation-mass spectrometry or rapid evaporative ionization mass spectrometry (REIMS). In a most preferred embodiment, the ambient spectroscopy technique is DESI-MS. Further details of the ambient spectroscopy techniques used herein can be found in WO 2016142691 and are herein incorporated by reference.

A key advantage of the method herein disclosed over previous technologies is the speed at which information on both the microbial composition and the inflammatory status of the subject can be obtained from the mucosal sample. This makes it particularly suited to point-of-care platforms and for use in environments where resources may be limited. Accordingly, the metabolic profile of the subject may be obtained within 10 minutes of the sample being obtained from the subject, preferably within 5 minutes of the sample being obtained from the subject, more preferably within 3 minutes of the sample being obtained from the subject. The metabolic profile of the subject may be obtained within 0-10 minutes, 1-10 minutes, 2-10 minutes, 3-10 minutes, 4-10 minutes, 5-10 minutes, 6-10 minutes, 7-10 minutes, 8-10 minutes, 9-10 minutes, 0-9 minutes, 1-9 minutes, 2-9 minutes, 3-9 minutes, 4-9 minutes, 5-9 minutes, 6-9 minutes, 7-9 minutes, 8-9 minutes, 0-8 minutes, 1-8 minutes, 2-8 minutes, 3-8 minutes, 4-8 minutes, 5-8 minutes, 6-8 minutes, 7-8 minutes, 0-7 minutes, 1-7 minutes, 2-7 minutes, 3-7 minutes, 4-7 minutes, 5-7 minutes, 6-7 minutes, 0-6 minutes, 1-6 minutes, 2-6 minutes, 3-6 minutes, 4-6 minutes, 5-6 minutes, 0-5 minutes, 1-5 minutes, 2-5 minutes, 3-5 minutes, 4-5 minutes, 0-4 minutes, 1-4 minutes, 2-4 minutes, 3-4 minutes, 0-3 minutes, 1-3 minutes, 2-3 minutes, 0-2 minutes, 1-2 minutes or 0-1 minute. In a most preferred embodiment, the metabolic profile of the subject is obtained within 3 minutes of the sample being obtained from the subject.

As previously outlined, the method herein described can be used to diagnose diseases/disorders in a subject, monitor the progression of said disease/disorder and used to inform the resulting treatment strategy for said subject. Accordingly, the subject's mucosal inflammatory state, level of mucosal immune activation and/or microbial composition may be indicative of an infection. Examples of such infections include, but are not limited to, respiratory infections, gastrointestinal infections, ear infections, nasal infections, throat infections, mouth infections, male reproductive tract infections, female reproductive tract infections, sexually transmitted diseases, HIV, bacterial vaginosis, candidiasis infection, and or HPV. In a preferred embodiment, the subject's mucosal inflammatory state, level of mucosal immune activation and/or microbial composition is indicative of a vaginal infection, for example, a sexually transmitted disease, BV, candidiasis infection, HPV or HIV.

The indicated infection may be a bacterial infection, a viral infection or a fungal infection, for example, a yeast infection. The bacteria may optionally be of a genus selected from, e.g., Abiotrophia, Achromobacter, Acidovorax, Acinetobacter, Actinobacillus, Actinomadura, Actinomyces, Aerococcus, Aeromonas, Anaerococcus, Anaplasma, Bacillus, Bacteroides, Bartonella, Bifidobacterium, Bordetella, Borrelia, Brevundimonas, Brucella, Burkholderia, Campylobacter, Capnocytophaga, Chlamydia, Citrobacter, Chlamydophila, Chryseobacterium, Clostridium, Comamonas, Corynebacterium, Coxiella, Cupriavidus, Delftia, Dermabacter, Ehrlichia, Eikenella, Enterobacter, Enterococcus, Escherichia, Erysipelothrix, Facklamia, Finegoldia, Francisella, Fusobacterium, Gemella, Gordonia, Haemophilus, Helicobacter, Klebsiella, Lactobacillus, Legionella, Leptospira, Listeria, Micrococcus, Moraxella, Morganella, Mycobacterium, Mycoplasma, Neisseria, Nocardia, Orientia, Pandoraea, Pasteurella, Peptoniphilus, Peptostreptococcus, Plesiomonas, Porphyromonas, Pseudomonas, Prevotella, Proteus, Propionibacterium, Rhodococcus, Ralstonia, Raoultella, Rickettsia, Rothia, Salmonella, Serratia, Shigella, Staphylococcus, Stenotrophomonas, Streptococcus, Tannerella, Treponema, Ureaplasma, Vibrio and/or Yersinia. The virus may optionally be selected from one or more of the Herpesviridae, optionally selected from Simplexvirus, Varicellovirus, Cytomegalovirus, Roseolovirus, Lymphocryptovirus, and/or Rhadinovirus; the Adenoviridae, optionally selected from Adenovirus and/or Mastadenovirus; Papillomaviridae, optionally selected from Alphapapillomavirus, Betapapillomavirus, Gammapapilloma-virus, Mupapillomavirus, and/or Nupapillomavirus; Polyomaviridae, optionally selected from Polyomavirus; Poxviridae, optionally selected from Molluscipoxvirus, Orthopoxvirus and/or Parapoxvirus; Anelloviridae, optionally selected from Alphatorquevirus, Betatorquevirus, and/or Gammatorquevirus; Mycodnaviridae, optionally selected from Gemycircular-viruses; Parvoviridae, optionally selected from Erythrovirus, Dependovirus, and/or Bocavirus; Reoviridae, optionally selected from Coltivirus, Rotavirus, and/or Seadornavirus; Coronaviridae, optionally selected from Alphacoronavirus, Betacoronavirus, and/or Torovirus; Astroviridae, optionally selected from Mamastrovirus; Caliciviridae, optionally selected from Norovirus, and/or Sapovirus; Flaviviridae, optionally selected from Flavivirus, Hepacivirus, and/or Pegivirus; Picornaviridae, optionally selected from Cardiovirus, Cosavirus, Enterovirus, Hepatovirus, Kobuvirus, Parechovirus, Rosavirus, and/or Salivirus; Togaviridae, optionally selected from Alphavirus and/or Rubivirus; Rhabdoviridae, optionally selected from Lyssavirus, and/or Vesiculovirus; Filoviridae optionally selected from Ebolavirus, and/or Marburgvirus; Paramyxoviridae, optionally selected from Henipavirus, Heffalumpvirus, Morbilivirus, Respirovirus, Rubulavirus, Metapneumovirus, and/or Pneumovirus; Arenaviridae, optionally selected from Arenavirus; Bunyaviridae, optionally selected from Hantavirus, Nairovirus, Orthobunyavirus, and/or Phlebovirus; Orthomyxoviridae, optionally selected from Influenzavirus A, Influenzavirus B, Influenzavirus C and/or Thogotovirus; Retroviridae, optionally selected from Gammaretrovirus, Deltaretrovirus, Lentivirus, Spumavirus; Epadnaviridae, optionally selected from Orthohepadnavirus; Hepevirus; and/or Deltavirus. The fungus may optionally be selected from the genus Aspergillus, Arthroascus, Brettanomyces Candida, Cryptococcus, Debaryomyces, Geotrichum, Pichia, Rhodotorula, Saccharomyces, Trichosporon and Zygotorulaspora.

In another embodiment, the subject's mucosal inflammatory state, level of mucosal immune activation and/or microbial composition may be indicative of key pregnancy outcomes. Accordingly, the subject's mucosal inflammatory state, level of mucosal immune activation and/or microbial composition may be indicative of cervical dysplasia, unsuccessful in vitro fertilization, miscarriage and/or pre-term birth. As such, the method herein described can be used to accurately, efficiently and robustly determine the inflammatory state/microbial composition of a sample obtained from a subject, which in turn can be used to make early medical decisions in a clinical setting to improve key pregnancy outcomes by determining the correct treatment plan, or altering the current treatment plan, for more favourable clinical outcomes. For example, rates of cervical dysplasia, unsuccessful in vitro fertilization, miscarriage and/or pre-term birth could be reduced using the method herein disclosed.

The method herein disclosed provides for a way in which a subject's mucosal inflammatory state, level of mucosal immune activation and/or the microbial composition can be monitored over a period of time.

Accordingly, in a second aspect, the present invention provides a method of monitoring the mucosal inflammatory state, level of mucosal immune activation and/or microbial composition of a subject, said method comprising any one of features herein disclosed, wherein the subject has previously had a sample analysed for one or more biomarkers, or any combination of biomarkers herein disclosed. As such, a single subject may have numerous samples obtained in order to perform the method herein disclosed and thus provide a way in which changes in the mucosal inflammatory state and/or microbial composition over a period of time can be monitored. For example, monitoring the subject in this manner may result in detection of improved clinical outcomes, or the detection of worsening of clinical outcomes, in which case the treatment strategy can be amended and the subject further monitored for signs of improvement. It is understood that the number of samples obtained from a subject will vary on a case-by-case basis and be dependent on various factors, for example, the treatment strategy, the disease/disorder to be treated/monitored, if symptoms are present, and the availability of the subject. In one embodiment, the subject from which the samples are to be obtained from may be a pregnant subject, with samples being collected and analysed using the method herein throughout the pregnancy. Preferably, the samples may be obtained on a monthly basis, even more preferably, the samples may be obtained on a biweekly or weekly basis depending on if the subject is categorised as a high-risk pregnant subject. Additional samples may be collected if the subject reports clinical symptoms or signs of infection (for example, discharge, itching, swelling and/or odour).

As the method herein disclosed may be used to monitor a subject's inflammatory status/microbial composition, the method herein disclosed may also be used to determine a suitable course of treatment for a disease/disorder. The disease/disorder to have a suitable course of treatment determined will be dependent on the mucosal sample obtained from the subject, for example, the disease/disorder may be a respiratory infection, a gastrointestinal infection, an ear infection, a nasal infection, a throat infection, a mouth infection, a male reproductive tract infection or a female reproductive tract infection. Preferably, the disease/disorder is in a female subject and may be a vaginal infection, for example, BV, candidiasis, HPV, HIV, or any other sexually transmitted disease, cervical dysplasia, unsuccessful in vitro fertilization, miscarriage and/or pre-term birth.

Accordingly, in a third aspect, the present invention provides a method of determining a suitable course of treatment for a subject having a vaginal infection, cervical dysplasia, unsuccessful in vitro fertilization, miscarriage and/or pre-term birth, the method comprising predicting the inflammatory state, level of mucosal immune activation and/or microbial composition of a subject using the method herein disclosed, wherein a suitable course of treatment is determined if the predicted inflammatory state, level of mucosal immune activation and/or microbial composition of the subject is indicative of vaginal infection, cervical dysplasia, unsuccessful in vitro fertilization, miscarriage and/or pre-term birth. The suitable course of treatment for each of the above will be apparent to the person skilled in the art. For example, in the case of a vaginal infection, the subject may be prescribed antibiotics, anti-viral therapies and/or probiotics.

Additionally, the method herein can be used to monitor the response of a subject to a specific treatment for a target disease/disorder. Accordingly, in a fourth aspect, the present invention is a method of monitoring the response of a subject to a treatment, said subject having a vaginal infection, cervical dysplasia, unsuccessful in vitro fertilization, miscarriage and/or pre-term birth, the method comprising predicting the inflammatory state, level of mucosal immune activation and/or microbial composition of a subject using the method herein disclosed, wherein a suitable course of treatment is determined if the predicted inflammatory state, level of mucosal immune activation and/or microbial composition of the subject is indicative of vaginal infection, cervical dysplasia, unsuccessful in vitro fertilization, miscarriage and/or pre-term birth.

The method herein disclosed can be used as a diagnostic assay to efficiently and reliably diagnose a disease/disorder associated with a mucosal membrane, for example, a bronchial mucosa membrane, a uterine mucosa membrane, an oesophageal mucosa membrane, a gastric mucosa membrane, an intestinal mucosa membrane, a nasal mucosa membrane, an olfactory mucosa membrane, an oral mucosa membrane, a penile mucosa membrane or a vaginal mucosa membrane. Preferably, the mucosal membrane is a vaginal mucosa membrane.

Accordingly, in a fifth aspect, the present invention provides a method of diagnosing a vaginal infection, cervical dysplasia, unsuccessful in vitro fertilisation, miscarriage and/or pre-term birth in a subject, wherein the method herein disclosed is performed on a sample obtained from the subject. The vaginal infection to be diagnosed may be a bacterial infection, a viral infection, a yeast infection and/or a sexually transmitted disease, as outlined above. More preferably, the vaginal infection is a BV infection, a candidiasis infection, a HPV infection, a HSV infection or a HIV infection.

In a sixth aspect, the present invention provides a method of treating a vaginal infection in a subject, said method comprising the steps of:(i) analysing a mucosal sample obtained from the subject to identify the presence and/or level of one or more biomarkers, or any combination of biomarkers, wherein the biomarkers are selected from a panel of biomarkers, wherein the panel of biomarkers comprises amino-aminohexyl-diaminomethylideneamino-pentanamide, aminonaphthalene, amino-sulfanylheptenyl-amino-oxooctadecanoic acid, arachidic acid (C20:0), arginine, asparagine, aspartic acid, butynoate, cadaverine, carboxysulfanyl-pentanedioic acid, Cer(d24:1/18:1), cerotic acid (C26:0), DG(36:3), dihydroxy-octadecadienoic acid, dimethoxyphenyl-hexadecylcarbamothioyl-propenamide, docosanoic acid (C22:0), docosanylsulfanylbutanoic acid, docosenoic acid (C22:1), eicosadienoic acid (C20:2), eicosanoic acid, ethyl-hexadecylcarbamothioylamino-oxobutanoate, galabiosylceramide (d18:1/16:0), galbeta-Cer(d40:1), glutamate, glutamine, glutamyl-leucine, glycyl-phenylalanine, heptadecylenic acid (C17:1), heptadecyl-hydroxyimidazole, hexadecenylsuccinic acid, histamine, HODE, hydroxycaproic acid, hydroxyglutaric acid, hydroxyvaleric acid, lactoylcysteine, leucine, leucyl-alanine, leucyl-glutamine, leucyl-glycyl-glycine, leucyl-serine, leucyl-threonine, lignoceric acid (C24:0), linoleic acid (C18:2), linolenic acid (C18:3), lysine, lyso-PE(16:1), lyso-PE(18:1), lyso-PE(18:2), lyso-PE(20:4), lyso-PE(O-16:0), lyso-PG(16:0), lyso-PS(18:0), maltotriose, methoxymethyl methyl-glucopyranosiduronate, methyl-hexadecylcarbamothioylamino-oxobutanoate, N2-acetyl-ornithine, N-acetylcadaverine, N-acetylputrescine, nervonic acid (C24:1), nonadecanoic acid (C19:0), nonadecenoic acid (C19:1), N-palmitoyl glutamine, N-palmitoyl methionine, N-palmitoyl-cysteine, octadecanoyloxamide, octadecanyloxy-propoxy-thiazolyl-propanamide, octadecenylamino-sulfanylpropanoic acid, octadecyl-oxy-octadecanoic acid (SAHSA), oleic acid (C18:1), oxo-octadecadienoic acid, oxoproline, oxosulfanyl-oxazolidinyl-octadecanamide, oxypurinol, PC(O-14:1/18:2), pentacosanoic acid (C25:0), PG(18:1/18:2), phenyl sulfoxide, phenylalanine, phenylethylamine, phenyllactic acid, phosphatidyl glycerol, phytenic acid (C20:1), PI(20:4/18:0), propyl-hexadecylcarbamothioylamino-oxobutanoate, PS(16:0/18:1), PS(18:0/18:1), putrescine, pyridinamine, pyrrolidinecarboxaldehyde, stearic acid (18:0), styrene, succinic acid, thiomalic acid, tolualdehyde, tricosanoic acid (C23:0), tyramine, vaccenyl carnitine, valyl-alanine, valyl-glutamine, valyl-phenylalanine, valyl-serine, vinylaniline, and ximenic acid (C26:1); (ii) forming a metabolic profile, wherein said metabolic profile is formed from data showing the presence and/or level of the biomarkers identified in step (i); (iii) using a predictive multivariate statistical model and/or a machine learning model pre-trained with metabolic and immuno-profiling datasets obtained from a reference population to predict the subject's mucosal inflammatory state and/or level of mucosal immune activation; and wherein the method further comprises the step of associating the metabolic profile of step ii) with metataxonomic data from a reference population to obtain the microbial composition of the mucosal sample, wherein when the subject's mucosal inflammatory state, level of mucosal immune activation and/or microbial composition are indicative of a vaginal infection, the subject is administered an anti-inflammatory agent, an antibiotic, a live biotherapeutic, a prebiotic, a probiotic, progesterone (P4) or undergoes a cervical cerclage procedure.

Examples of such anti-inflammatory agents include, but are not limited to, ibuprofen, naproxen, diclofenac, celecoxib, mefenemic acid, etoricoxibb, indomethacin and aspirin. Examples of suitable antibiotics include, but are not limited to, metronidazole, clindamycin and tinidazole. Examples of live biotherapuetics/probiotics include, but are not limited to, oral or vaginal administration of commensal vaginal bacterial strains including but not limited to Lactobacillus crispatus strains (e.g. CTV-005, Lactin V).

In one embodiment, the method of treating a vaginal infection in a subject comprises analysing a mucosal sample obtained from the subject to identify the presence and/or level of one or more biomarkers, or any combination of biomarkers, wherein the subject's mucosal inflammatory state and/or level of mucosal immune activation may be predicted using biomarkers selected from a panel of biomarkers comprising amino-sulfanylheptenyl-amino-oxooctadecanoic acid, Cer(d24:1/18:1), dimethoxyphenyl-hexadecylcarbamothioyl-propenamide, docosanoic acid docosenoic (C22:0), acid (C22:1), ethyl-hexadecylcarbamothioylamino-oxobutanoate, galabiosylceramide (d18:1/16:0), galbeta-Cer(d40:1), heptadecyl-hydroxyimidazole, hexadecenylsuccinic acid, lactoylcysteine, methyl-hexadecylcarbamothioylamino-oxobutanoate, N-palmitoyl glutamine, octadecanoyloxamide, oxosulfanyl-oxazolidinyl-octadecanamide, phytenic acid (C20:1), and PI(20:4/18:0), or any combination thereof.

The panel of biomarkers may further comprise amino-aminohexyl-diaminomethylideneamino-pentanamide, aminonaphthalene, arachidic acid (C20:0), arginine, asparagine, aspartic acid, butynoate, cadaverine, carboxysulfanyl-pentanedioic acid, cerotic acid (C26:0), DG(36:3), dihydroxy-octadecadienoic acid, docosanylsulfanylbutanoic acid, eicosadienoic acid (C20:2), eicosanoic acid, glutamate, glutamine, glutamyl-leucine, glycyl-phenylalanine, heptadecylenic acid (C17:1), histamine, HODE, hydroxycaproic acid, hydroxyglutaric acid, hydroxyvaleric acid, leucine, leucyl-alanine, leucyl-glutamine, leucyl-glycyl-glycine, leucyl-serine, leucyl-threonine, lignoceric acid (C24:0), linoleic acid (C18:2), linolenic acid (C18:3), lysine, lyso-PE(16:1), lyso-PE(18:1), lyso-PE(18:2), lyso-PE(20:4), lyso-PE(O-16:0), lyso-PG(16:0), lyso-PS(18:0), maltotriose, methoxymethyl methyl-glucopyranosiduronate, N2-acetyl-ornithine, N-acetylcadaverine, N-acetylputrescine, nervonic acid (C24:1), nonadecanoic acid (C19:0), nonadecenoic acid (C19:1), N-palmitoyl methionine, octadecanyloxy-propoxy-thiazolyl-propanamide, N-palmitoyl-cysteine, octadecenylamino-sulfanylpropanoic acid, octadecyl-oxy-octadecanoic acid (SAHSA), oleic acid (C18:1), oxo-octadecadienoic acid, oxoproline, oxypurinol, PC(O-14:1/18:2), pentacosanoic acid (C25:0), PG(18:1/18:2), phenyl sulfoxide, phenylalanine, phenylethylamine, phenyllactic acid, phosphatidyl glycerol, propyl-hexadecylcarbamothioylamino-oxobutanoate, PS(16:0/18:1), PS(18:0/18:1), putrescine, pyridinamine, pyrrolidinecarboxaldehyde, stearic acid (18:0), styrene, succinic acid, thiomalic acid, tolualdehyde, tricosanoic acid (C23:0), tyramine, vaccenyl carnitine, valyl-alanine, valyl-glutamine, valyl-phenylalanine, valyl-serine, vinylaniline, and ximenic acid (C26:1), or any combinations thereof.

In another embodiment, the method of treating a vaginal infection in a subject comprises analysing a mucosal sample obtained from the subject to identify the presence and/or level of one or more biomarkers, or any combination of biomarkers, wherein the microbial composition of the mucosal sample may be predicted using biomarkers selected from a panel of biomarkers comprising amino-sulfanylheptenyl-amino-oxooctadecanoic acid, dimethoxyphenyl-hexadecylcarbamothioyl-propenamide, docosanoic acid (C22:0), docosenoic acid (C22:1), eicosadienoic acid (C20:2), ethyl-hexadecylcarbamothioylamino-oxobutanoate, hydroxycaproic acid, hydroxyvaleric acid, leucyl-serine, lignoceric acid (C24:0), nonadecanoic acid (C19:0), nonadecenoic acid (C19:1), N-palmitoyl-cysteine, oxosulfanyl-oxazolidinyl-octadecanamide, phytenic acid (C20:1), thiomalic acid, and tricosanoic acid (C23:0), or any combination thereof.

The panel of biomarkers may further comprise amino-aminohexyl-diaminomethylideneamino-pentanamide, aminonaphthalene, arachidic acid (C20:0), arginine, asparagine, aspartic acid, butynoate, cadaverine, carboxysulfanyl-pentanedioic acid, Cer(d24:1/18:1), cerotic acid (C26:0), DG(36:3), dihydroxy-octadecadienoic acid, docosanylsulfanylbutanoic acid, eicosanoic acid, galabiosylceramide (d18:1/16:0), galbeta-Cer(d40:1), glutamate, glutamine, glutamyl-leucine, glycyl-phenylalanine, heptadecylenic acid (C17:1), heptadecyl-hydroxyimidazole, hexadecenylsuccinic acid, histamine, HODE, hydroxyglutaric acid, lactoylcysteine, leucine, leucyl-alanine, leucyl-glutamine, leucyl-glycyl-glycine, leucyl-threonine, linoleic acid (C18:2), linolenic acid (C18:3), lysine, lyso-PE(16:1), lyso-PE(18:1), lyso-PE(18:2), lyso-PE(20:4), lyso-PE(O-16:0), lyso-PG(16:0), lyso-PS(18:0), maltotriose, methoxymethyl methyl-glucopyranosiduronate, methyl-hexadecylcarbamothioylamino-oxobutanoate, N2-acetyl-ornithine, N-acetylcadaverine, N-acetylputrescine, nervonic acid (C24:1), N-palmitoyl glutamine, N-palmitoyl methionine, octadecanoyloxamide, octadecanyloxy-propoxy-thiazolyl-propanamide, octadecenylamino-sulfanylpropanoic acid, octadecyl-oxy-octadecanoic acid (SAHSA), oleic acid (C18:1), oxo-octadecadienoic acid, oxoproline,, oxypurinol, PC(O-14:1/18:2), pentacosanoic acid (C25:0), PG(18:1/18:2), phenyl sulfoxide, phenylalanine, phenylethylamine, phenyllactic acid, phosphatidyl glycerol, PI(20:4/18:0), propyl-hexadecylcarbamothioylamino-oxobutanoate, PS(16:0/18:1), PS(18:0/18:1), putrescine, pyridinamine, pyrrolidinecarboxaldehyde, stearic acid (18:0), styrene, succinic acid, tolualdehyde, tyramine, vaccenyl carnitine, valyl-alanine, valyl-glutamine, valyl-phenylalanine, valyl-serine, vinylaniline, and ximenic acid (C26:1), or any combination thereof.

In a seventh aspect, the present invention provides a method of preventing pre-term birth in a subject, said method comprising the steps of: (i) analysing a mucosal sample obtained from the subject to identify the presence and/or level of one or more biomarkers, or any combination of biomarkers, wherein the biomarkers are selected from a panel of biomarkers, wherein the panel of biomarkers comprises amino-aminohexyl-diaminomethylideneamino-pentanamide, aminonaphthalene, amino-sulfanylheptenyl-amino-oxooctadecanoic acid, arachidic acid (C20:0), arginine, asparagine, aspartic acid, butynoate, cadaverine, carboxysulfanyl-pentanedioic acid, Cer(d24:1/18:1), cerotic acid (C26:0), DG(36:3), dihydroxy-octadecadienoic acid, dimethoxyphenyl-hexadecylcarbamothioyl-propenamide, docosanoic acid (C22:0), docosanylsulfanylbutanoic acid, docosenoic acid (C22:1), eicosadienoic acid (C20:2), eicosanoic acid, ethyl-hexadecylcarbamothioylamino-oxobutanoate, galabiosylceramide (d18:1/16:0), galbeta-Cer(d40:1), glutamate, glutamine, glutamyl-leucine, glycyl-phenylalanine, heptadecylenic acid (C17:1), heptadecyl-hydroxyimidazole, hexadecenylsuccinic acid, histamine, HODE, hydroxycaproic acid, hydroxyglutaric acid, hydroxyvaleric acid, lactoylcysteine, leucine, leucyl-alanine, leucyl-glutamine, leucyl-glycyl-glycine, leucyl-serine, leucyl-threonine, lignoceric acid (C24:0), linoleic acid (C18:2), linolenic acid (C18:3), lysine, lyso-PE(16:1), lyso-PE(18:1), lyso-PE(18:2), lyso-PE(20:4), lyso-PE(O-16:0), lyso-PG(16:0), lyso-PS(18:0), maltotriose, methoxymethyl methyl-glucopyranosiduronate, methyl-hexadecylcarbamothioylamino-oxobutanoate, N2-acetyl-ornithine, N-acetylcadaverine, N-acetylputrescine, nervonic acid (C24:1), nonadecanoic acid (C19:0), nonadecenoic acid (C19:1), N-palmitoyl glutamine, N-palmitoyl methionine, N-palmitoyl-cysteine, octadecanoyloxamide, octadecanyloxy-propoxy-thiazolyl-propanamide, octadecenylamino-sulfanylpropanoic acid, octadecyl-oxy-octadecanoic acid (SAHSA), oleic acid (C18:1), oxo-octadecadienoic acid, oxoproline, oxosulfanyl-oxazolidinyl-octadecanamide, oxypurinol, PC(O-14:1/18:2), pentacosanoic acid (C25:0), PG(18:1/18:2), phenyl sulfoxide, phenylalanine, phenylethylamine, phenyllactic acid, phosphatidyl glycerol, phytenic acid (C20:1), PI(20:4/18:0), propyl-hexadecylcarbamothioylamino-oxobutanoate, PS(16:0/18:1), PS(18:0/18:1), putrescine, pyridinamine, pyrrolidinecarboxaldehyde, stearic acid (18:0), styrene, succinic acid, thiomalic acid, tolualdehyde, tricosanoic acid (C23:0), tyramine, vaccenyl carnitine, valyl-alanine, valyl-glutamine, valyl-phenylalanine, valyl-serine, vinylaniline, and ximenic acid (C26:1); (ii) forming a metabolic profile, wherein said metabolic profile is formed from data showing the presence and/or level of the biomarkers identified in step (i); (iii) using a predictive multivariate statistical model and/or a machine learning model pre-trained with metabolic and immuno-profiling datasets obtained from a reference population to predict the subject's mucosal inflammatory state and/or level of mucosal immune activation; and wherein the method further comprises the step of associating the metabolic profile of step ii) with metataxonomic data from a reference population to obtain the microbial composition of the mucosal sample, wherein when the subject's mucosal inflammatory state, level of mucosal immune activation and/or microbial composition are indicative of pre-term birth, the subject is administered a preventative therapy or is advised to undergo a preventative procedure.

Examples of preventative therapies and procedures herein disclosed include, but are not limited to, progesterone therapy, cervical cerclage, cervical pessary (e.g. Arabin pessary) and/or targeted antibiotic therapy.

In one embodiment, the method of preventing pre-term birth in a subject comprises analysing a mucosal sample obtained from the subject to identify the presence and/or level of one or more biomarkers, or any combination of biomarkers, wherein the subject's mucosal inflammatory state and/or level of mucosal immune activation may be predicted using biomarkers selected from a panel of biomarkers comprising amino-sulfanylheptenyl-amino-oxooctadecanoic acid, Cer(d24:1/18:1), dimethoxyphenyl-hexadecylcarbamothioyl-propenamide, docosanoic acid (C22:0), docosenoic acid (C22:1), ethyl-hexadecylcarbamothioylamino-oxobutanoate, galabiosylceramide (d18:1/16:0), galbeta-Cer(d40:1), heptadecyl-hydroxyimidazole, hexadecenylsuccinic acid, lactoylcysteine, methyl-hexadecylcarbamothioylamino-oxobutanoate, N-palmitoyl glutamine, octadecanoyloxamide, oxosulfanyl-oxazolidinyl-octadecanamide, phytenic acid (C20:1), and PI(20:4/18:0), or any combination thereof.

The panel of biomarkers may further comprise amino-aminohexyl-diaminomethylideneamino-pentanamide, aminonaphthalene, arachidic acid (C20:0), arginine, asparagine, aspartic acid, butynoate, cadaverine, carboxysulfanyl-pentanedioic acid, cerotic acid (C26:0), DG(36:3), dihydroxy-octadecadienoic acid, docosanylsulfanylbutanoic acid, eicosadienoic acid (C20:2), eicosanoic acid, glutamate, glutamine, glutamyl-leucine, glycyl-phenylalanine, heptadecylenic acid (C17:1), histamine, HODE, hydroxycaproic acid, hydroxyglutaric acid, hydroxyvaleric acid, leucine, leucyl-alanine, leucyl-glutamine, leucyl-glycyl-glycine, leucyl-serine, leucyl-threonine, lignoceric acid (C24:0), linoleic acid (C18:2), linolenic acid (C18:3), lysine, lyso-PE(16:1), lyso-PE(18:1), lyso-PE(18:2), lyso-PE(20:4), lyso-PE(O-16:0), lyso-PG(16:0), lyso-PS(18:0), maltotriose, methoxymethyl methyl-glucopyranosiduronate, N2-acetyl-ornithine, N-acetylcadaverine, N-acetylputrescine, nervonic acid (C24:1), nonadecanoic acid (C19:0), nonadecenoic acid (C19:1),, N-palmitoyl methionine, N-palmitoyl-cysteine, octadecanyloxy-propoxy-thiazolyl-propanamide, octadecenylamino-sulfanylpropanoic acid, octadecyl-oxy-octadecanoic acid (SAHSA), oleic acid (C18:1), oxo-octadecadienoic acid, oxoproline, oxypurinol, PC(O-14:1/18:2), pentacosanoic acid (C25:0), PG(18:1/18:2), phenyl sulfoxide, phenylalanine, phenylethylamine, phenyllactic acid, phosphatidyl glycerol, propyl-hexadecylcarbamothioylamino-oxobutanoate, PS(16:0/18:1), PS(18:0/18:1), putrescine, pyridinamine, pyrrolidinecarboxaldehyde, stearic acid (18:0), styrene, succinic acid, thiomalic acid, tolualdehyde, tricosanoic acid (C23:0), tyramine, vaccenyl carnitine, valyl-alanine, valyl-glutamine, valyl-phenylalanine, valyl-serine, vinylaniline, and ximenic acid (C26:1), or any combinations thereof.

In one embodiment, the method of preventing pre-term birth in a subject comprises analysing a mucosal sample obtained from the subject to identify the presence and/or level of one or more biomarkers, or any combination of biomarkers, wherein the microbial composition of the mucosal sample may be predicted using biomarkers selected from a panel of biomarkers comprising amino-sulfanylheptenyl-amino-oxooctadecanoic acid, dimethoxyphenyl-hexadecylcarbamothioyl-propenamide, docosanoic acid (C22:0), docosenoic acid (C22:1), eicosadienoic acid (C20:2), ethyl-hexadecylcarbamothioylamino-oxobutanoate, hydroxycaproic acid, hydroxyvaleric acid, leucyl-serine, lignoceric acid (C24:0), nonadecanoic acid (C19:0), nonadecenoic acid (C19:1), N-palmitoyl-cysteine, oxosulfanyl-oxazolidinyl-octadecanamide, phytenic acid (C20:1), thiomalic acid, and tricosanoic acid (C23:0), or any combination thereof.

The panel of biomarkers may further comprise amino-aminohexyl-diaminomethylideneamino-pentanamide, aminonaphthalene, arachidic acid (C20:0), arginine, asparagine, aspartic acid, butynoate, cadaverine, carboxysulfanyl-pentanedioic acid, Cer(d24:1/18:1), cerotic acid (C26:0), DG(36:3), dihydroxy-octadecadienoic acid, docosanylsulfanylbutanoic acid, eicosanoic acid, galabiosylceramide (d18:1/16:0), galbeta-Cer(d40:1), glutamate, glutamine, glutamyl-leucine, glycyl-phenylalanine, heptadecylenic acid (C17:1), heptadecyl-hydroxyimidazole, hexadecenylsuccinic acid, histamine, HODE, hydroxyglutaric acid, lactoylcysteine, leucine, leucyl-alanine, leucyl-glutamine, leucyl-glycyl-glycine, leucyl-threonine, linoleic acid (C18:2), linolenic acid (C18:3), lysine, lyso-PE(16:1), lyso-PE(18:1), lyso-PE(18:2), lyso-PE(20:4), lyso-PE(O-16:0), lyso-PG(16:0), lyso-PS(18:0), maltotriose, methoxymethyl methyl-glucopyranosiduronate, methyl-hexadecylcarbamothioylamino-oxobutanoate, N2-acetyl-ornithine, N-acetylcadaverine, N-acetylputrescine, nervonic acid (C24:1), N-palmitoyl glutamine, N-palmitoyl methionine, octadecanoyloxamide, octadecanyloxy-propoxy-thiazolyl-propanamide, octadecenylamino-sulfanylpropanoic acid, octadecyl-oxy-octadecanoic acid (SAHSA), oleic acid (C18:1), oxo-octadecadienoic acid, oxoproline,, oxypurinol, PC(O-14:1/18:2), pentacosanoic acid (C25:0), PG(18:1/18:2), phenyl sulfoxide, phenylalanine, phenylethylamine, phenyllactic acid, phosphatidyl glycerol, PI(20:4/18:0), propyl-hexadecylcarbamothioylamino-oxobutanoate, PS(16:0/18:1), PS(18:0/18:1), putrescine, pyridinamine, pyrrolidinecarboxaldehyde, stearic acid (18:0), styrene, succinic acid, tolualdehyde, tyramine, vaccenyl carnitine, valyl-alanine, valyl-glutamine, valyl-phenylalanine, valyl-serine, vinylaniline, and ximenic acid (C26:1), or any combination thereof.

Accordingly, the methods herein disclosed allow for rapid detection of vaginal microbiome compositions during pregnancy, which in turn could permit identification of women at risk of preterm birth and may optimize selection of preventative interventions. Such stratification strategies could be extended to targeted antibiotic treatment following preterm premature rupture of the fetal membranes and assessment of suitability for vaginal seeding at birth for the partial restoration of the gut microbiota of caesarean-born infants in addition to monitoring efficacy and host response of treatments designed to optimize vaginal microbiome composition, such as live biotherapeutics and vaginal microbiome transplantation.

In an eighth aspect, the present invention provides a kit comprising a first device arranged and adapted to direct a spray of charged droplets onto a surface of a swab in order to generate a plurality of analyte ions; a second device arranged and adapted to analyse said analyte ions, wherein said analyte ions are analysed for the presence and/or level of one or more biomarkers, or any combination of biomarkers, wherein the biomarkers are selected from a panel of biomarkers, wherein the panel of biomarkers comprises amino-aminohexyl-diaminomethylideneamino-pentanamide, aminonaphthalene, amino-sulfanylheptenyl-amino-oxooctadecanoic acid, arachidic acid (C20:0), arginine, asparagine, aspartic acid, butynoate, cadaverine, carboxysulfanyl-pentanedioic acid, Cer(d24:1/18:1), cerotic acid (C26:0), DG(36:3), dihydroxy-octadecadienoic acid, dimethoxyphenyl-hexadecylcarbamothioyl-propenamide, docosanoic acid (C22:0), docosanylsulfanylbutanoic acid, docosenoic acid (C22:1), eicosadienoic acid (C20:2), eicosanoic acid, ethyl-hexadecylcarbamothioylamino-oxobutanoate, galabiosylceramide (d18:1/16:0), galbeta-Cer(d40:1), glutamate, glutamine, glutamyl-leucine, glycyl-phenylalanine, heptadecylenic acid (C17:1), heptadecyl-hydroxyimidazole, hexadecenylsuccinic acid, histamine, HODE, hydroxycaproic acid, hydroxyglutaric acid, hydroxyvaleric acid, lactoylcysteine, leucine, leucyl-alanine, leucyl-glutamine, leucyl-glycyl-glycine, leucyl-serine, leucyl-threonine, lignoceric acid (C24:0), linoleic acid (C18:2), linolenic acid (C18:3), lysine, lyso-PE(16:1), lyso-PE(18:1), lyso-PE(18:2), lyso-PE(20:4), lyso-PE(O-16:0), lyso-PG(16:0), lyso-PS(18:0), maltotriose, methoxymethyl methyl-glucopyranosiduronate, methyl-hexadecylcarbamothioylamino-oxobutanoate, N2-acetyl-ornithine, N-acetylcadaverine, N-acetylputrescine, nervonic acid (C24:1), nonadecanoic acid (C19:0), nonadecenoic acid (C19:1), N-palmitoyl glutamine, N-palmitoyl methionine, N-palmitoyl-cysteine, octadecanoyloxamide, octadecanyloxy-propoxy-thiazolyl-propanamide, octadecenylamino-sulfanylpropanoic acid, octadecyl-oxy-octadecanoic acid (SAHSA), oleic acid (C18:1), oxo-octadecadienoic acid, oxoproline, oxosulfanyl-oxazolidinyl-octadecanamide, oxypurinol, PC(O-14:1/18:2), pentacosanoic acid (C25:0), PG(18:1/18:2), phenyl sulfoxide, phenylalanine, phenylethylamine, phenyllactic acid, phosphatidyl glycerol, phytenic acid (C20:1), PI(20:4/18:0), propyl-hexadecylcarbamothioylamino-oxobutanoate, PS(16:0/18:1), PS(18:0/18:1), putrescine, pyridinamine, pyrrolidinecarboxaldehyde, stearic acid (18:0), styrene, succinic acid, thiomalic acid, tolualdehyde, tricosanoic acid (C23:0), tyramine, vaccenyl carnitine, valyl-alanine, valyl-glutamine, valyl-phenylalanine, valyl-serine, vinylaniline, and ximenic acid (C26:1).

In one embodiment, the kit may comprise biomarkers selected from a panel of biomarkers comprising amino-sulfanylheptenyl-amino-oxooctadecanoic acid, Cer(d24:1/18:1), dimethoxyphenyl-hexadecylcarbamothioyl-propenamide, docosanoic acid (C22:0), acid docosenoic (C22:1), ethyl-hexadecylcarbamothioylamino-oxobutanoate, galabiosylceramide (d18:1/16:0), galbeta-Cer(d40:1), heptadecyl-hydroxyimidazole, hexadecenylsuccinic acid, lactoylcysteine, methyl-hexadecylcarbamothioylamino-oxobutanoate, N-palmitoyl glutamine, octadecanoyloxamide, oxosulfanyl-oxazolidinyl-octadecanamide, phytenic acid (C20:1), and PI(20:4/18:0), or any combination thereof.

The panel of biomarkers may further comprise amino-aminohexyl-diaminomethylideneamino-pentanamide, aminonaphthalene, arachidic acid (C20:0), arginine, asparagine, aspartic acid, butynoate, cadaverine, carboxysulfanyl-pentanedioic acid, cerotic acid (C26:0), DG(36:3), dihydroxy-octadecadienoic acid, docosanylsulfanylbutanoic acid, eicosadienoic acid (C20:2), eicosanoic acid, glutamate, glutamine, glutamyl-leucine, glycyl-phenylalanine, heptadecylenic acid (C17:1), histamine, HODE, hydroxycaproic acid, hydroxyglutaric acid, hydroxyvaleric acid, leucine, leucyl-alanine, leucyl-glutamine, leucyl-glycyl-glycine, leucyl-serine, leucyl-threonine, lignoceric acid (C24:0), linoleic acid (C18:2), linolenic acid (C18:3), lysine, lyso-PE(16:1), lyso-PE(18:1), lyso-PE(18:2), lyso-PE(20:4), lyso-PE(O-16:0), lyso-PG(16:0), lyso-PS(18:0), maltotriose, methoxymethyl methyl-glucopyranosiduronate, N2-acetyl-ornithine, N-acetylcadaverine, N-acetylputrescine, nervonic acid (C24:1), nonadecanoic acid (C19:0), nonadecenoic acid (C19:1), N-palmitoyl methionine, N-palmitoyl-cysteine, octadecanyloxy-propoxy-thiazolyl-propanamide, octadecenylamino-sulfanylpropanoic acid, octadecyl-oxy-octadecanoic acid (SAHSA), oleic acid (C18:1), oxo-octadecadienoic acid, oxoproline, oxypurinol, PC(O-14:1/18:2), pentacosanoic acid (C25:0), PG(18:1/18:2), phenyl sulfoxide, phenylalanine, phenylethylamine, phenyllactic acid, phosphatidyl glycerol, propyl-hexadecylcarbamothioylamino-oxobutanoate, PS(16:0/18:1), PS(18:0/18:1), putrescine, pyridinamine, pyrrolidinecarboxaldehyde, stearic acid (18:0), styrene, succinic acid, thiomalic acid, tolualdehyde, tricosanoic acid (C23:0), tyramine, vaccenyl carnitine, valyl-alanine, valyl-glutamine, valyl-phenylalanine, valyl-serine, vinylaniline, and ximenic acid (C26:1), or any combinations thereof.

In another embodiment, the kit may comprise biomarkers selected from a panel comprising amino-sulfanylheptenyl-amino-oxooctadecanoic acid, dimethoxyphenyl-hexadecylcarbamothioyl-propenamide, docosanoic acid (C22:0), docosenoic acid (C22:1), eicosadienoic acid (C20:2), ethyl-hexadecylcarbamothioylamino-oxobutanoate, hydroxycaproic acid, hydroxyvaleric acid, leucyl-serine, lignoceric acid (C24:0), nonadecanoic acid (C19:0), nonadecenoic acid (C19:1), N-palmitoyl-cysteine, oxosulfanyl-oxazolidinyl-octadecanamide, phytenic acid (C20:1), thiomalic acid, and tricosanoic acid (C23:0), or any combination thereof.

The panel of biomarkers may further comprise amino-aminohexyl-diaminomethylideneamino-pentanamide, aminonaphthalene, arachidic acid (C20:0), arginine, asparagine, aspartic acid, butynoate, cadaverine, carboxysulfanyl-pentanedioic acid, Cer(d24:1/18:1), cerotic acid (C26:0), DG(36:3), dihydroxy-octadecadienoic acid, docosanylsulfanylbutanoic acid, eicosanoic acid, galabiosylceramide (d18:1/16:0), galbeta-Cer(d40:1), glutamate, glutamine, glutamyl-leucine, glycyl-phenylalanine, heptadecylenic acid (C17:1), heptadecyl-hydroxyimidazole, hexadecenylsuccinic acid, histamine, HODE, hydroxyglutaric acid, lactoylcysteine, leucine, leucyl-alanine, leucyl-glutamine, leucyl-glycyl-glycine, leucyl-threonine, linoleic acid (C18:2), linolenic acid (C18:3), lysine, lyso-PE(16:1), lyso-PE(18:1), lyso-PE(18:2), lyso-PE(20:4), lyso-PE(O-16:0), lyso-PG(16:0), lyso-PS(18:0), maltotriose, methoxymethyl methyl-glucopyranosiduronate, methyl-hexadecylcarbamothioylamino-oxobutanoate, N2-acetyl-ornithine, N-acetylcadaverine, N-acetylputrescine, nervonic acid (C24:1), N-palmitoyl glutamine, N-palmitoyl methionine, octadecanoyloxamide, octadecanyloxy-propoxy-thiazolyl-propanamide, octadecenylamino-sulfanylpropanoic acid, octadecyl-oxy-octadecanoic acid (SAHSA), oleic acid (C18:1), oxo-octadecadienoic acid, oxoproline, oxypurinol, PC(O-14:1/18:2), pentacosanoic acid (C25:0), PG(18:1/18:2), phenyl sulfoxide, phenylalanine, phenylethylamine, phenyllactic acid, phosphatidyl glycerol, PI(20:4/18:0), propyl-hexadecylcarbamothioylamino-oxobutanoate, PS(16:0/18:1), PS(18:0/18:1), putrescine, pyridinamine, pyrrolidinecarboxaldehyde, stearic acid (18:0), styrene, succinic acid, tolualdehyde, tyramine, vaccenyl carnitine, valyl-alanine, valyl-glutamine, valyl-phenylalanine, valyl-serine, vinylaniline, and ximenic acid (C26:1), or any combination thereof.

The inventors of the present invention have surprisingly found that the methods herein disclosed allow for the simultaneous measurement of both mucosal membrane microbes and immune responses within the same clinical sample, thus leading to improved clinical outcomes.

The invention is further described with reference to the following non-limiting examples:

### EXAMPLES

Details of the methods used in the examples herein are found below:

### Study Subjects and Sample Collection

The study was conducted with approval of the NHS National Research Ethics Service (NRES) Committees London-City and East (REC 12/LO/2003) and London-Stanmore (REC 14/LO/0328), and by the North of Scotland Research Ethics Service (REC 14/NS/1078). All participants provided written informed consent prior to sampling and experiments were performed in accordance with the approved institutional guidelines. Recruitment and sampling was performed at the Queen Charlotte's and Chelsea, St Mary's and Chelsea & Westminster Hospitals (Imperial College Healthcare NHS Trust, London, UK), University College London Hospital (NHS Trust, London, UK) and the Royal Infirmary of Edinburgh, Scotland, UK). Eligibility criteria were pregnant women with a singleton pregnancy, with and without risk factors for preterm birth. Exclusion criteria included women under 18 years of age, sexual intercourse within 72 h of sampling, vaginal bleeding in the preceding week, antibiotic use in the preceding 2 weeks, multiple pregnancies, HIV or Hepatitis C positive status. Detailed maternal outcome and clinical metadata were collected for all participants. Cervicovaginal fluid swab samples were collected at clinical visits throughout pregnancy from the posterior fornix using BBL CultureSwab MaxV Liquid Amies swabs (Becton, Dickinson and Company, Oxford, UK), placed in either Amies transport media or a sterile eppendorf on ice, before storage at −80° C. within 5 min of collection.

### Metabolic Profiling of Cervicovaginal Swabs Using Direct Swab Analysis by DESI-MS

All chemicals used were analytical reagent grade. HPLC grade methanol and water for DESI-MS analysis and swab sample extraction were purchased from Sigma-Aldrich (St Louis, MO). The method used for the direct analysis of vaginal swab samples by DESI MS is detailed elsewhere⁴. Briefly, we used a LTQ-Orbitrap Discovery mass spectrometer (Thermo Scientific, Bremen, Germany) coupled with a DESI-MS source designed for direct swab analysis. Swabs were placed into a rotating holder positioned orthogonally in front of the MS inlet capillary with a swab-capillary distance of approximately 2 mm. The DESI sprayer tip was pointed to the swab center with a tip-sample distance of 1.5-2 mm and an altitude difference between the tip and the inlet capillary of 2 mm. The entire surface of the medical swabs was analyzed by DESI-MS through clockwise rotation of the swab toward the MS capillary. The cervicovaginal mucosa was absorbed from the swab tip by gently desorbing the analytes with charged droplets of methanol/water (95:5, v/v) mixture and directed to the mass spectrometer. For each sample, 30 scan mass spectra (m/z 50-1000, R=30.000 (FWHM)) were recorded in the negative and positive ion mode.

### Metabolic Profiling of Cervicovaginal Swabs Using Direct Swab Analysis by LC-MS Analysis

Liquid extraction was performed on each swab by adding a MeOH:H₂O (1:1, v:v) solution as eluent to a final concentration of 50 mg vaginal fluid/ml. Each blank swab was extracted with 1 ml solution using a repeated sonication and vortexing cycle for 30 sec each. Recovery of soluble material was achieved through centrifugation of swabs (2000×g for 2 minutes) seated in a 200 μl loading tip positioned in a sterile Eppendorf tube. Associated supernants were pooled and centrifuged at 16000×g for 10 min to remove insoluble material before the resulting supernatant was divided into 3 aliquots. An additional extraction of lipids from the swab by repeating the procedure with a isopropanol:water (4:1, v:v) solution to a final extraction of 25 mg vaginal fluid/ml. The resulting extract was pooled with one aliquot of the methanol:water extract. All extracts were then evaporated using a SpeedVac for further reconstitution before analysis by LC-MS^{5,6}.

### Reversed Phase LC-MS Analysis of Small Metabolites

Sample reconstitution was performed in 300 μl of water. 250 μl aliquot of reconstituted sample material were used for individual sample preparation of 96 well plates including the addition of 25 μl of full RP 2× labelled standard mix (L-glutamine-13C₅; L-glutamic acid ¹³C₅; creatinine-methyl-D₃; cytidine-5,6-D₂; citric acid ¹³C₆; L-isoleucine-¹³C₆¹⁵N; L-leucine-¹³C₆; L-phenylalanine-¹³C₉¹⁵N; hippuric acid-D₅, benzoic acid-¹³C₆, octanoic acid-¹³C₈, L-tryptophane-¹³C₁₁¹⁵N₂). In addition, 50 μl aliquot of each sample were used for pooling and generation of quality control sample. For chromatographic separation a 2 μl aliquot of extracted metabolites from each sample was injected onto a reverse-phase 150×2.1 mm ACQUITY 1.8-μm High Strength Silica (HSS) column (Waters Corp.) kept at 45° C. using an ACQUITY UPLC system (Waters Corp.). The mobile phase consisting of 0.1% formic acid in water (A) and acetonitrile containing 0.1% formic acid (B). Each sample was resolved for 12.65 minutes at a flow rate of 0.5 ml/minute. The gradient consisted of 99% A and 1% B for 0.1 minutes, then a ramp of curve 6 to 100% B from 0.1 minutes to 10.70 minutes.

### Reversed Phase LC-MS Analysis for Lipids

Sample reconstitution was performed in 300 μl of water/isopropanol (4:1). 250 μl aliquot of reconstituted sample material were used for individual sample preparation of 96 well plates including the addition of full RP labelled standard mix (C17:0; LPC(6:0/0:0); LPC(9:0/0:0); LPC(15:0/0:0); PC(11:0/11:0); PC(15:0/15:0); PE(15:0/15:0); PA(17:0/17:0); PG(15:0/15:0); PS(17:0/17:0); SM(d18:1/17:0); Cer(d18:1/17:0); DG(19:0/0:0/19:0); PC(23:0/23:0); TG(8:0/8:0/8:0); TG(10:0/10:0/10:0); TG(12:0/12:0/12:0); TG(14:0/14:0/14:0); TG(15:0/15:0/15:0); TG(16:0/16:0/16:0); TG(17:0/17:0/17:0); DG(18:0/20:4/0:0); DG(18:0/18:0)). In addition, 50 μl aliquot of each sample were used for pooling and generation of quality control sample. For chromatographic separation a 2 μl aliquot of extracted metabolites from each sample was injected onto a Waters Acquity UPLC BEH C8, 1.7 μm, 2.1×100 mm column (Waters Corp.) kept at 55° C. using an ACQUITY UPLC system (Waters Corp.). The mobile phase consisting of water:isopropanol:acetonitrile (50:25:25)+5 mM ammonium acetate+0.05% acetic acid+20 μM phosphoric acid (A) and isopropanol:acetonitrile 50:50+5 mM ammonium acetate+0.05% acetic acid (B). Each sample was resolved for 13.15 minutes at a flow rate of 0.5 ml/minute. Starting conditions were 99% A and 1% B and the gradient changed with a ramp of curve 6 as follows: decrease to 70% A and 30% B over the first 2 min; decrease to 10% A with 90% B from 2 to 11.50 min, decrease to 0.1% A with 99.9% B from 11.50 to 12.50 min, after which the solvent composition returned to starting conditions over 0.1 min until 13.15 min.

### HILIC LC-MS Analysis

Sample reconstitution was performed in 200 μl of water:acetonitrile mixture (1:3.6, v:v). 115 μl aliquot of reconstituted sample material were used for individual sample preparation of 96 well plates including the addition of 2.5 μl of 48× labelled IS standard mix ((uracil-2-¹³C¹⁵N₂; N-benzoyl-d₅-glycine, adenosine-2-D₁, adenine-2-D₁, taurine-¹⁵N, L-tryptophan-D₅, L-phenylalanine-¹³C₉¹⁵N, creatine-(methyl-D3)-monohydrate, L-arginine-¹³C₆-hydrochloride). In addition, 20 μl aliquot of each sample were used for pooling and generation of quality control sample. HILIC chromatography analysis was performed using a Waters Acquity UPLC BEH HILIC (1.7 μm, 2.1×150 mm) column (Waters Corporation, Milford, MA, USA) kept at 40° C. The mobile phases consisted of 0.1% formic acid and 20 mM ammonium formate in water (A), and 0.1% formic acid in acetonitrile (B) at a flow rate of 0.5 mL/min. Starting conditions were 5% A and 95% B and the gradient changed with a ramp of curve 6 as follows:increase to 20% A and 80% B over the first 5.5 min; increase to 50% A with 50% B from 5.5 to 7.1 min, after which the solvent composition returned to starting conditions over 0.1 min until 12.65 min. The injection volume was 2 μl of extracted metabolites for negative ion mode analysis.

### LC-MS Instrumental Operation and Analysis

The column eluent was introduced directly into the mass spectrometer by electrospray. MS was performed on a Waters Xevo G2-S QTOF mass spectrometer (Waters Ltd., Elstree, UK) operating in either negative-ion (ESI−) or positive-ion (ESI+) electrospray ionization mode with a capillary voltage of 1 kV for RP and 1.5 kV for HILIC, and a sampling cone voltage of 20 V. The desolvation gas flow was set to 1000 L/h and the desolvation temperature was set to 600° C. The cone gas flow was 150 L/h, and the source temperature was 120° C. Accurate mass was maintained by introduction of LockSpray interface of Leucine Enkephalin (m/z 556.2771 in ESI+, m/z 554.2615 in ESI−) at a concentration of 200 pg/μl in 50% aqueous acetonitrile with a scan time of 0.07 seconds over 4 scans, after each interval of 60 seconds. Data were collected in centroid mode from 50 to 1200 m/z in MS scanning mode. To ensure system suitability and stability, a study reference (SR) quality control (QC) sample was prepared by combining equal aliquots of all the samples and injected at regular intervals throughout the analytical run. This SR sample was also used to condition the column (30 injections) prior to the analysis of both the ESI+ and ESI mode batches. Blank samples (i.e., injection of the reconstitution solvent) were also run to check the presence of artefact or contaminant peaks. A dilution series of the SR sample was also acquired at the beginning and end of injection sequence. Data-dependent acquisition (DDA) and MSE analysis of the SR sample were performed for structural elucidation.

### Mass Spectral Data Processing

DESI-MS raw mass spectral data were converted from.raw files into .mzXML format via the ProteoWizard msConverterGUI⁷. The first 30 spectra per sample were averaged into a single spectrum and saved as .csv file using the MALDIquant R package. The detailed parameter settings and algorithm used in the R package MALDIquant⁸ are as follow: Peaks were aligned with a half window size set to 20, tolerance set to 0.002, signal-to-noise-ratio (SNR) set to 2 using the warping method LOWESS. Peak detection was performed by setting SNR to 3 and half window size to 10 (using median absolute deviation (MAD) method). Then the reference peaks were created by calling the function “referencePeaks” from MALDIquant with method “strict”, “minFrequency” set to 0.01 and peak binning tolerance 0.002.

Raw LC-MS data files were first converted to the .mzML open format the Proteowizard msconvert. .mzML files were processed in R(v4.0.3) using the XCMS (v3.10.2) package⁹. Peaks were detected with the centWave algorithm (ppm=25, mzdiff=0.001, prefilter=c(4, 1000), noise=100, snthresh=5), and then grouped with the ‘density’ method (bw=3, mzwid=0.0007, minFrac-0.4). The centWave peakwidth parameter was set depending on the chromatographic method (c(1.5, 14) for HILIC-LC-MS (−) c(3, 12) in RP-LC-MS Lipid (+/−) and c(1.5, 5) in the RP-LC-MS Metabolite (+/−). Non-detected peaks were filled with fillPeaks, using the default arguments, and no retention time correction was done. The XCMS datasets were further filtered and the feature intensities drift corrected in Python (v3.8.5) using the nPYc-Toolbox (v1.2.4)¹⁰. Drift correction was performed using LOWESS trend-line model fitted on the SR samples. Features where the coefficient of variation measured on repeated injections of the SR sample was larger than 30% or whose Pearson correlation with dilution was less than 0.7 (measured using the SR dilution series) were excluded from the data matrix.

### Metabolite Identification

Target m/z features were putatively annotated using online databases including HMDB, Metlin, MMCD, KEGG, and Lipidmaps with a 5 ppm tolerance for each compound. To account for potential imprecision in m/z measurement due to peak binning and data processing artefacts, raw m/z value were confirmed by inspection of the.raw data. Metabolite annotation was performed by searching the measured m/z ratios against METLIN (http://metlin.scripps.edu), Lipidmaps (http://www.lipidmaps.org), and the HMDB (http://www.hmdb.ca)^{11,12,13}. The mass error used was 5 ppm. Further structural elucidation was performed using MS/MS experiments via collision-induced dissociation on the LTQ-Discovery MS instrument (Thermo Scientific), and with data-dependent analysis (DDA) of the precursor ion by the quadrupole of the Xevo G2 XS Q-TOF mass spectrometer (Waters corporation). For the annotation of metabolites the MS/MS spectra were matched against spectral libraries from HMDB, NIST and METLIN that were compiled with either authentic standards or theoretical assignment. Identification of metabolites where MS/MS reference spectra were not available were annotated using chemical fragmentation rules. For the structural assignment of glycerophospholipid, fragments of the polar head group or the fatty acyl chains were investigated to confirm the annotation proposed by the databases and discriminate isomers. MSI levels of compound identification were further reported as suggested by the Metabolomics Standards Initiative (MSI)¹⁴.

### DNA Extraction and Sequencing of 16S rRNA Amplicons

Extraction of bacterial DNA was performed as previously described¹⁵. For the VMET cohort, V1-V3 hypervariable regions of bacterial 16S rRNA genes were amplified using a forward and reverse fusion primer. The forward primer was constructed with the Illumina 15 adapter (5′-AATGATACGGCGACCACCGAGATCTACAC-3′) (SEQ ID NO:1), an 8-base pair (bp) bar code, a primer pad (forward, 5′-TATGGTAATT-3′) (SEQ ID NO:2), and the 28F primer (5′-GAGTTTGATCNTGGCTCAG-3′) (SEQ ID NO:3) (64). The reverse fusion primer consisted of the Illumina i7 adapter (5′-CAAGCAGAAGACGGCATACGAGAT-3′) (SEQ ID NO:4), an 8-bp bar code, a primer pad (reverse, 5′-AGTCAGTCAG-3′) (SEQ ID NO:5), and the 519R primer (5′-GTNTTACNGCGGCKGCTG-3′) (SEQ ID NO:6). For the VMET2 cohort, the V1-V2 hyper variable regions were amplified with the forward primer set (28f-YM) consisting of a mixture of the following primers mixed at a 4:1:1:1 ratio; 28F-Borrellia GAGTTTGATCCTGGCTTAG (SEQ ID NO:7); 28F-Chlorflex NO:8); 28F-Bifido GAATTTGATCTTGGTTCAG (SEQ ID GGGTTCGATTCTGGCTCAG (SEQ ID NO:9); 28F GAGTTTGATCNTGGCTCAG (SEQ ID NO:10). The reverse primer consisted of; 388R TGCTGCCTCCCGTAGGAGT (SEQ ID NO:11)¹⁶. Sequencing was performed at RTL Genomics (Lubbock, TX, USA) using an Illumina MiSeq platform (Illumina Inc.). Resulting sequence data were analyzed using the MiSeq SOP Pipeline of the Mothur package¹⁷. The Silva bacterial database (www.arb-silva.de/) was used for sequence alignment and classification was performed using the RDP (Ribosomal Database Project) database reference sequence files¹⁸. Determination of operational taxonomic unit taxonomies (phylum to genus) and species-level taxonomies was performed with USEARCH with 16S rRNA gene sequences from the cultured representatives from the RDP database¹⁹. Species-level taxonomies was complemented using information from the STIRRUPS database²⁰.

Counts from all OTUs assigned to the same species were summed to generate matrices of total counts per species. Species with less than 50 total counts were excluded. Community state types were then assigned to each sample with hierarchical clustering, using Ward-linkage and the Jensen-Shannon distance. The number of clusters which maximized the mean silhouette score were selected. The relative abundance of each species in a cluster was inspected and the clusters matched to CST reported in previous publications. Samples where the most abundant species was a Lactobacillus spp other than L. crispatus, L. iners, L. gasseri or L. jensenni, where manually assigned to a separate cluster (CST VII). Hierarchical clustering analyses and heatmaps were performed using Python (v3.8.5), ‘scipy’²¹ (v1.5.2), the ‘matplotlib’²² (v3.3.2) and ‘seaborn’²³ (v0.11.0) libraries.

### Immune/Inflammatory Profiling of Cervicovaginal Samples

Swabs were thawed on ice and re-suspended in 350 ml of phosphate-buffered saline solution containing protease inhibitor (5 □L/ml; Sigma-Aldrich) before being centrifuged at 7000×g for 10 min to to remove cellular debris. Customised multiplex assays (R&D Systems) were then used together with a Bio-Plex 200 system (Bio-Rad Laboratories Ltd.) to quantify levels of IL-1β, IL-2, IL-4, IL-5, IL-6, IL-8, IL-10, IL-18, IFN-γ, GM-CSF and TNF-α, with a 10-fold dilution of 1L-8 performed using Calibrator Diluent RD6-52 prior to assaying. The Human Complement Magnetic Bead panel 1 (Milliplex®) was used for measurement of C5, C5a and MBL, whilst panel 2 was used for measurement of C3b. Levels of immunglobulins IgA, IgG1, IgG2, IgG3, IgG4 and IgM were assessed using the ProcartaPlex Human Antibody Isotyping Panel 7plex assay (ThermoFisher Scientific).

### DESI-MS Profiling of Bacterial Isolates

Bacterial isolates analysed in this study were derived from the DSMZ-German Collection of Microorganism and Cell Culture GmbH (DSM number, Methods Table 1) or clinical samples received by the Imperial College NHS Healthcare Trust Diagnostic Microbiology laboratory (NHS number, Methods Table 1) at Charing Cross Hospital, London, after the completion of standard identification workflows. Identification of isolates from clinical samples was performed using a MALDI Biotyper instrument (Bruker, UK)²⁴. Isolates were stored on beads and in glycerol broth at −80° C. The bacterial culture library consisted of Atopobium vaginae, Bifidobacterium breve, Gardnerella vaginalis, Lactobacillus crispatus (5× isolates), Lactobacillus gasseri (5× isolates), Lactobacillus iners, Lactobacillus gasseri (5× isolates), Lactobacillus jensenii (5× isolates), Prevotella amni, Prevotella disiens, Prevotella timonensis, Staphylococcus aureus, Streptococcus agalactiae and Streptococcus anginosus. Microorganisms were grown from either fresh cultures or from beads with each isolate cultured 10 times using optimal culture conditions (Methods Table 1).

METHODS TABLE 1
Bacterial isolates and culture conditions used for DESI-MS analysis

| | | | Temp | | Length | | | MALDI |
|---|---|---|---|---|---|---|---|---|
| Isolate # | Genera | Species | (° C.) | Atmosphere | (h) | Media | Specimen Type | score |
| DSM 15829 | Atopobium | Atopobium vaginae | 37 | Anaerobic | 48 | CBA | Vagina | NA |
| DSM 20213 | Bifidobacterium | Bifidobacterium breve | 37 | Anaerobic | 48 | BA | Intestine of infant | NA |
| DSM 4944 | Gardenella | Gardnerella vaginalis | 37 | Anaerobic | 48 | CBA | Vagina | NA |
| NHS 219 | Lactobacillus | Lactobacillus crispatus | 37 | CO2 | 48 | CBA | Vagina | 2.20 |
| NHS 1712 | Lactobacillus | Lactobacillus crispatus | 37 | CO2 | 48 | CBA | Vagina | 2.06 |
| NHS 1713 | Lactobacillus | Lactobacillus crispatus | 37 | CO2 | 48 | CBA | Vagina | 1.86 |
| NHS 1757 | Lactobacillus | Lactobacillus crispatus | 37 | CO2 | 48 | CBA | Vagina | 1.83 |
| NHS 1759 | Lactobacillus | Lactobacillus crispatus | 37 | CO2 | 48 | CBA | Vagina | 1.88 |
| NHS 401 | Lactobacillus | Lactobacillus gasseri | 37 | CO2 | 48 | CBA | Vagina | 2.32 |
| NHS 902 | Lactobacillus | Lactobacillus gasseri | 37 | CO2 | 48 | CBA | Vagina | 2.36 |
| NHS 1272 | Lactobacillus | Lactobacillus gasseri | 37 | CO2 | 48 | CBA | Vagina | 2.38 |
| NHS 1588 | Lactobacillus | Lactobacillus gasseri | 37 | CO2 | 48 | CBA | Vagina | 1.75 |
| NHS 1758 | Lactobacillus | Lactobacillus gasseri | 37 | CO2 | 48 | CBA | Vagina | 1.93 |
| DSM 13335 | Lactobacillus | Lactobacillus iners | 37 | Anaerobic | 48 | MRS | Human urine | NA |
| NHS 1339 | Lactobacillus | Lactobacillus jensenii | 37 | CO2 | 48 | CBA | Endocervical | 2.00 |
| NHS 1342 | Lactobacillus | Lactobacillus jensenii | 37 | CO2 | 48 | CBA | Vagina | 2.14 |
| NHS 1589 | Lactobacillus | Lactobacillus jensenii | 37 | CO2 | 48 | CBA | Vagina | 2.10 |
| NHS 1693 | Lactobacillus | Lactobacillus jensenii | 37 | CO2 | 48 | CBA | Vagina | 2.42 |
| NHS 2672 | Lactobacillus | Lactobacillus jensenii | 37 | CO2 | 48 | CBA | Vagina | 1.83 |
| DSM 23384 | Prevotella | Prevotella amni | 37 | Anaerobic | 48 | FAA | Amniotic fluid | NA |
| NHS 987 | Prevotella | Prevotella disiens | 37 | Anaerobic | 48 | CBA | Gastrointestinal tract | 2.2 |
| DSM 22865 | Prevotella | Prevotella timonensis | 37 | Anaerobic | 48 | FAA | Human breast abscess | NA |
| NHS 3836 | Staphylococcus | Staphylococcus aureus | 37 | Aerobic | 24 | CBA | Blood | 2.37 |
| NHS 3929 | Streptococcus | Streptococcus agalactiae | 37 | CO2 | 48 | CBA | Blood | 2.02 |
| NHS 4165 | Streptococcus | Streptococcus anginosus | 37 | CO2 | 48 | CBA | Blood | 2.03 |

Bacterial isolates were sampled directly from solid agar plates using a medical rayon swab before being transferred into a sterile Eppendorf tube and stored at −80° C. Three “blank” swab samples of each solid agar media were also collected. Direct swab analysis using DESI-MS was performed in both positive and negative ion mode. Spectra were processed and assembled into a single data matrix. For each isolate, the intensities per m/z feature of biological replicates were compared against their corresponding culture media background samples using the Wilcoxon-Mann-Whitney test. The list of significant features was matched to the set of identified features from the analysis of human cervicovaginal swab samples. The mean fold change for matched peaks (tolerance <5 ppm) was estimated as the ratio of the mean intensity within bacterial biomass samples to the mean intensity in the background culture media samples. Heatmaps were generated in Python (v3.8.5), with the ‘matptlotlib’ (v3.3.2) and ‘seaborn’ (v0.11.0) libraries.

### Statistical Analysis of Metabolomic Profiling Data

The linear mixed effect modelling analyses were performed in R using the ‘Ime4’ package. For all metabolic profile variables in each data matrix, a linear mixed effect model with the following ‘Ime4’²⁵ formula was fitted: Metabolite ˜GestationalAge+CST+MaternalAge+BMI+Ethnicity+(0+GestationalAge|SubjectID)+(1|SubjectID). This model structure accounts for the repeated measures by using a random intercept per pregnancy and a random slope per pregnancy. No random effect term was used to model correlation between the random slopes and random intercepts. Gestational age, CST, Age, BMI and Ethnicity were modelled as fixed effects. All models were fitted using restricted maximum likelihood (‘REML=TRUE’ in Imer function call). The ‘emmeans’²⁶ package (v1.5.2.1) was used to perform contrast coding and obtain effect size estimates and p-values for contrasts and trends of interest. For the comparisons between CST's, the mean levels and contrasts were estimated assuming a maternal age=30, BMI=23 and gestational age=20, and averaged over all ethnicities. For the gestational age trend estimates were further averaged across all CSTs. The comparisons between Lactobacillus dominant vs depleted was encoded as a comparison between the grand mean of CST I, II III, V and VII levels versus the mean of CST IV. The p-values for each contrast tested were calculated using the Kenward-Roger approximation, implemented in the ‘pbkrtest’ package (v0.4.8.6)²⁷. The p-values for all contrasts within a single metabolic signal were not corrected for multiple testing. Instead, for each of the main contrasts of interest (early gestation vs late gestation, CST I vs III, CST I vs IV, CST III vs IV and LD vs NLD), all p-values obtained across all metabolic variables in an assay were pooled and FDR corrected together as a signature, using Benjamini-Hochberg false discovery rate correction and selecting a 5% FDR cut-off. Heatmaps were created in Python (v3.8.5), ‘matptlotlib’ (v3.3.2) and ‘seaborn’ (v0.11.0). Only features which were statistically significant after false discovery rate correction and replicated in both VMET and VMET2 datasets were plotted. To account for differences in m/z calibration between datasets, a feature was only considered to be replicated between datasets if it was possible to find a marker with an m/z error of less than 5 part-per-million in the final FDR corrected signature from the other study.

For the immune-metabolite association analysis, a series of linear models with the formula Im(log(ImmuneMarker+1)˜Metabolite) were fitted in R(v4.0.3) for all immune-marker cytokine pair combinations, and the corresponding F-test p-values calculated. Benjamini-Hochberg false discovery rate correction was used independently for each immune marker analysis, with a 5% FDR cut-off. Heatmaps were generated using Python (v3.8.5), ‘matplotlib’ (v3.3.2) and ‘seaborn’ (v0.11.0), using the results of the linear model analysis and the random forest regression models (detailed in the next section).

### Prediction of CST and Immune Markers From the Metabolomic Data

The CST type was predicted from the metabolomic profiles using random forest classifiers. For the Lactobacillus dominant versus depleted comparison, samples which were assigned to CST I, II, III, V or VII were assigned to the Lactobacillus dominant class, and samples with CST IV or VI label assigned to Lactobacillus depleted. Random forest classifiers were then trained to predict the CST or Lactobacillus depletion status using the metabolic profile variables, without using any other clinical or demographical variables.

For the immune marker prediction, immune marker concentrations were initially log-transformed after addition of a constant offset log(x+1). A random forest regressor was trained to predict a single log-transformed immune marker level. All random forest models were trained in R(v4.0.3)²⁸, using the ‘randomForest’ (v4.6-14) package in combination with the ‘caret’ package (v6.0-86)²⁹, for model fitting, performance metric calculation and cross-validation. RF classifiers were trained using constant default parameters:number of trees ‘ntree’=1000, and the number of variables ‘mtry=number of metabolic variables/3’. For the random forest regression models, ‘mtry=sqrt (number of metabolic variables’. A repeated (n=15) stratified 5-fold cross validation procedure was used for all models. This ensures that the train and test sets contain a similar proportion of samples from each class.

The performance of the RF models was assessed using only data left out from each CV round (CV test sets). For the classifiers, ROC curves, precision-recall curves, accuracy, sensitivity, specificity, positive-predictive value and negative predictive values were calculated and plotted with the R packages ‘caret’, ‘pROC’ (v1.16.2)³⁰, ‘plotROC’ (v2.2.1)³¹, ‘precrec’ (v0.11.2)³² and ‘ggplot2’ (v3.3.2)³³. For the regression analysis, the R-squared was calculated with ‘caret’ and predictions plotted with ‘ggplot2’.

### Example 1

### Direct On-Swab Metabolic Profiling of Cervicovaginal Fluid by DESI-MS Enables Robust Prediction of Major Microbiome Compositions

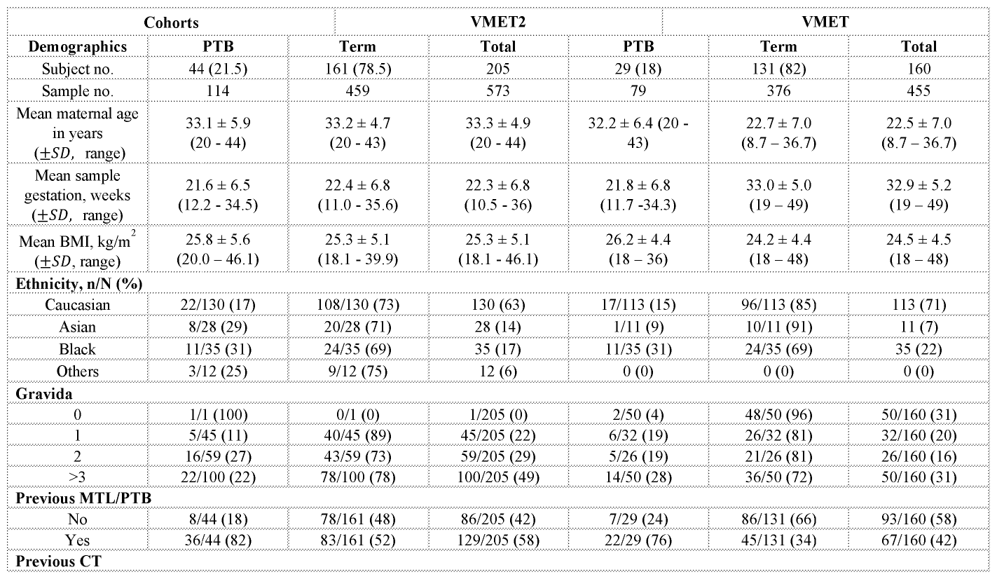

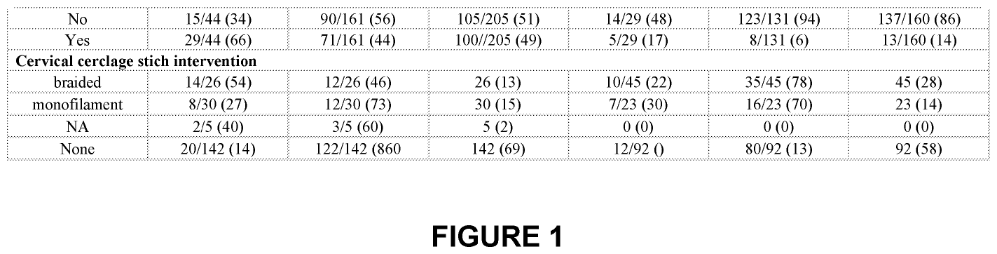

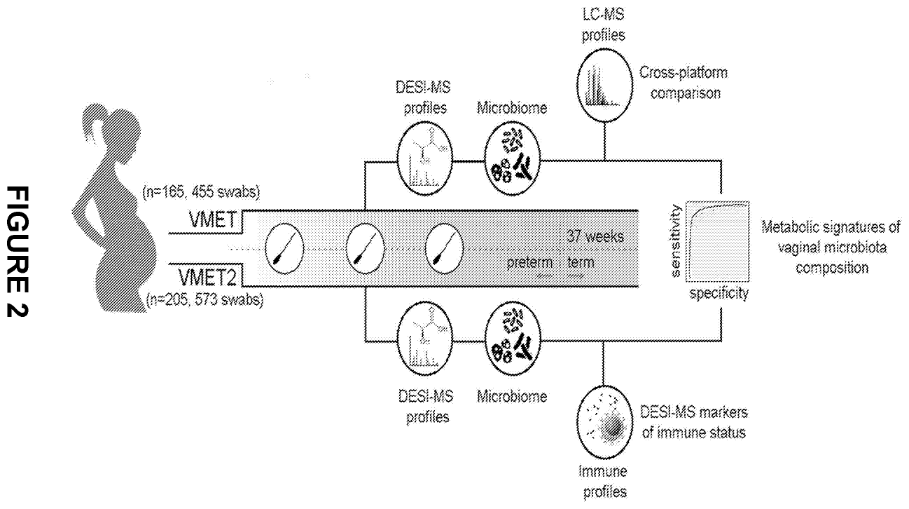

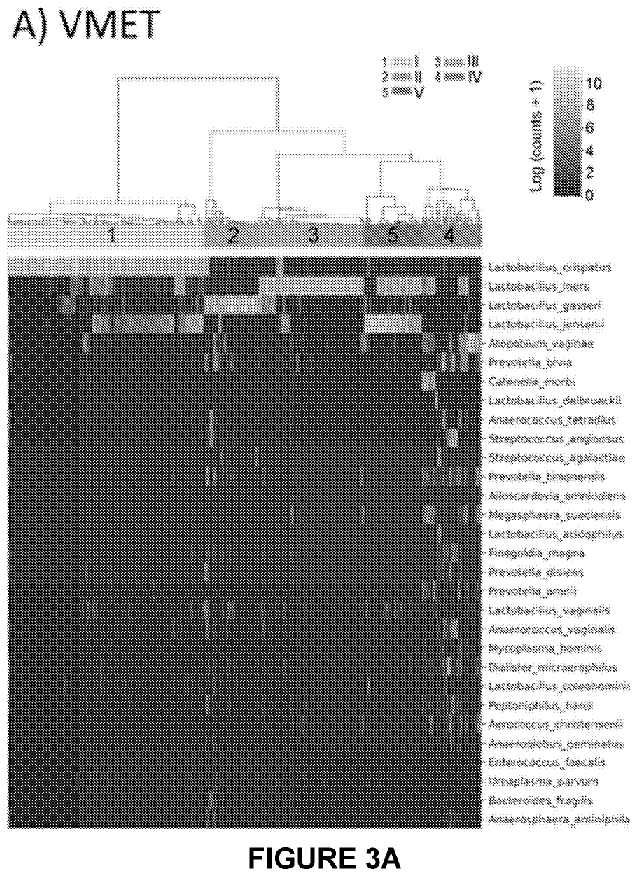

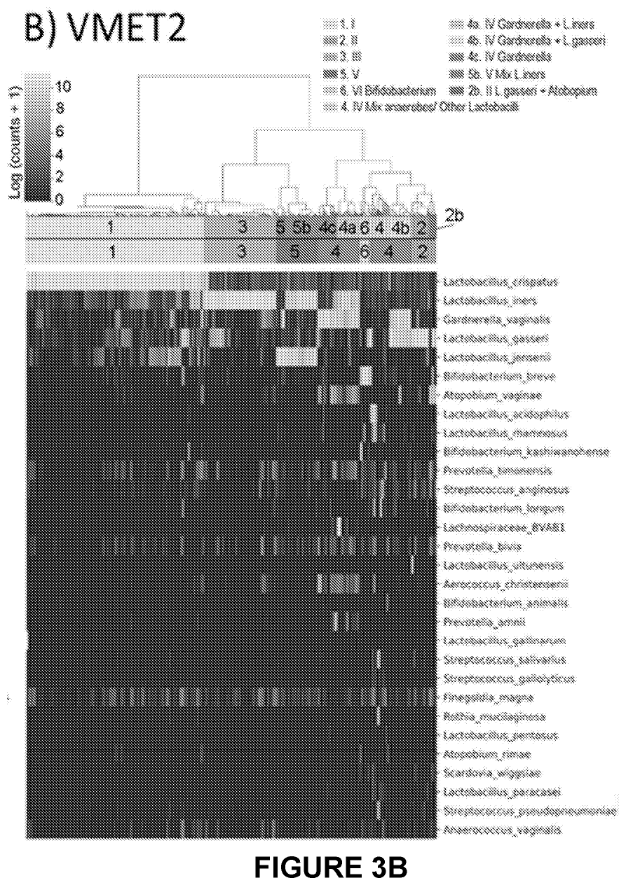

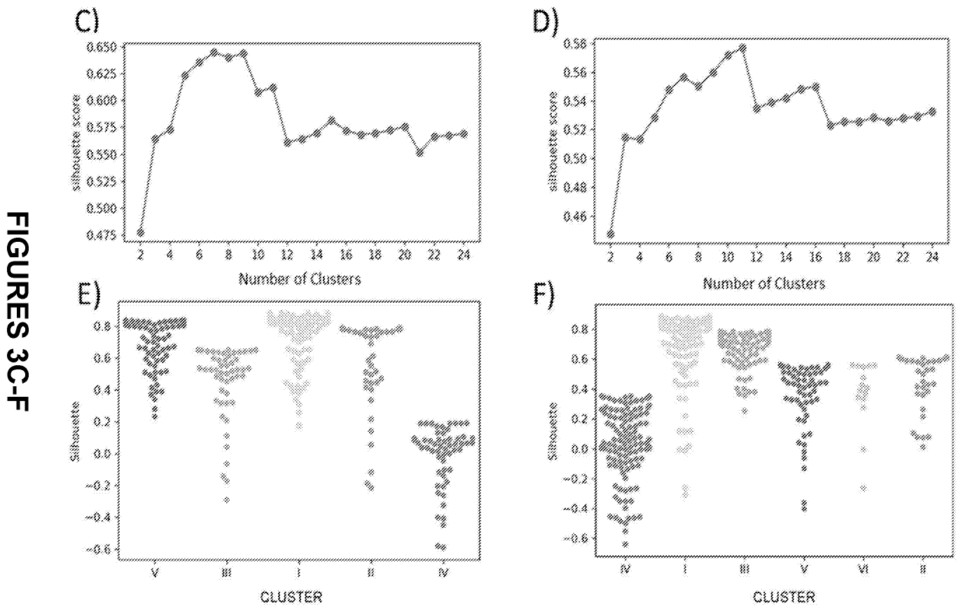

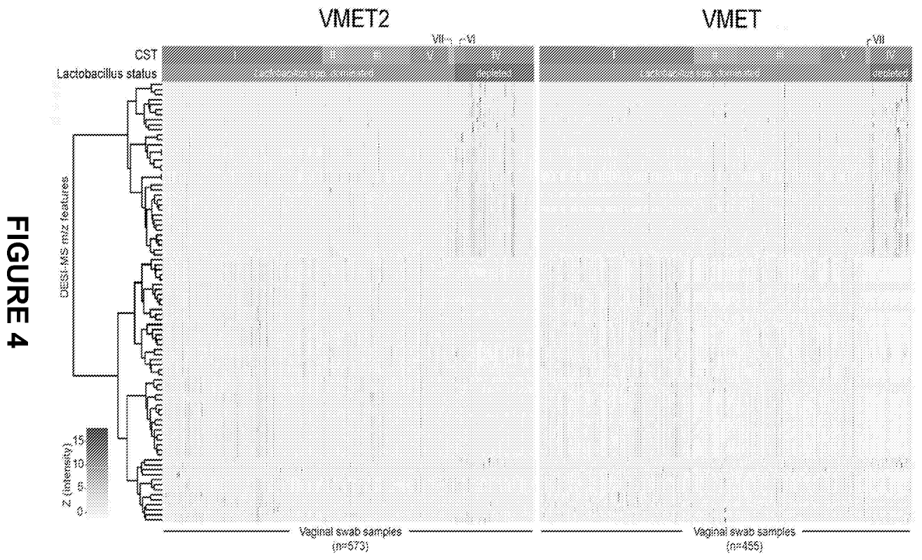

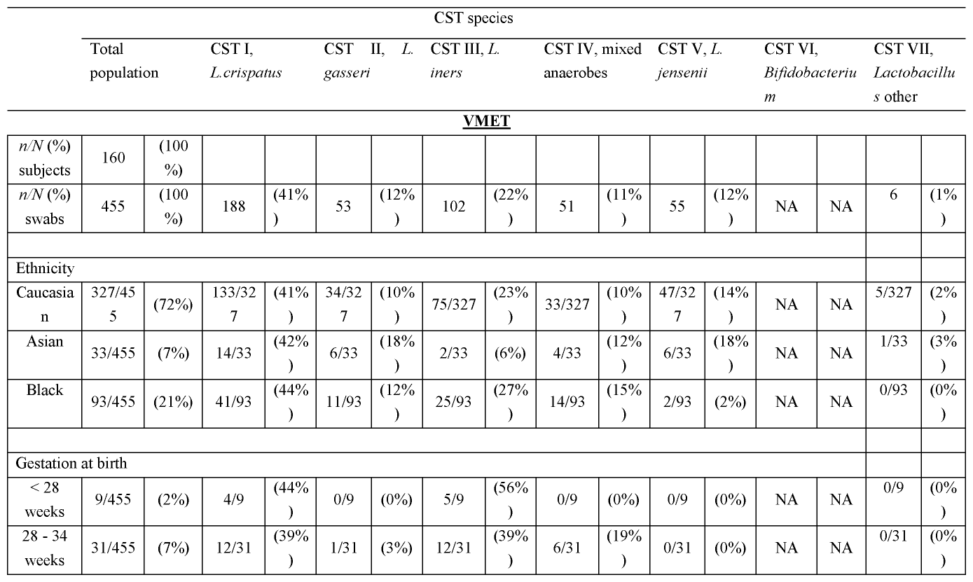

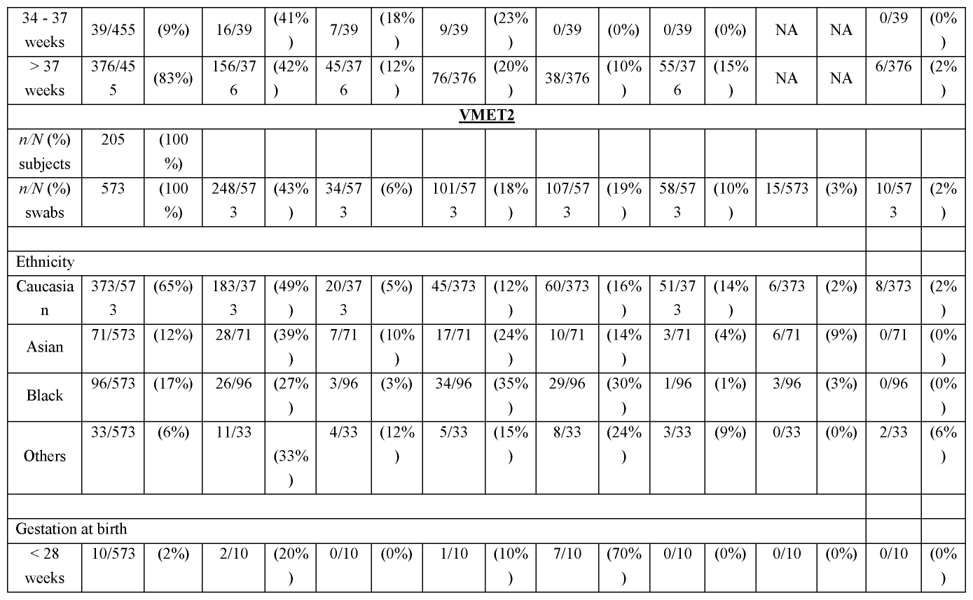

DESI-MS was applied to vaginal swabs collected throughout pregnancy from two independent cohorts of clinically phenotyped women at high risk of preterm birth (VMET, n=160 women; 455 swabs; VMET II, n=205; 573 swabs, FIG. 1). Cervicovaginal metabolic profiles were acquired within 3 minutes/sample using direct on-swab DESI-MS metabolic profiling and characterization of the bacterial composition of the vaginal microbiome was performed using MiSeq-based sequencing of 16S rRNA gene amplicons^{34,35,36}. Cross-platform comparison of DESI-MS metabolic profiling data was achieved using five complementary LC-MS assays (FIG. 2). Ward's linkage hierarchical clustering (HC) analysis with Jensen-Shannon distance metrics of bacterial species was used to group vaginal samples into 11 distinct groups as evaluated with Silhouette scores (FIG. 3) that were reduced into community state types (CSTs) consistent with previous studies (FIG. 4)^{35, 36, 37,38, 39,40}.

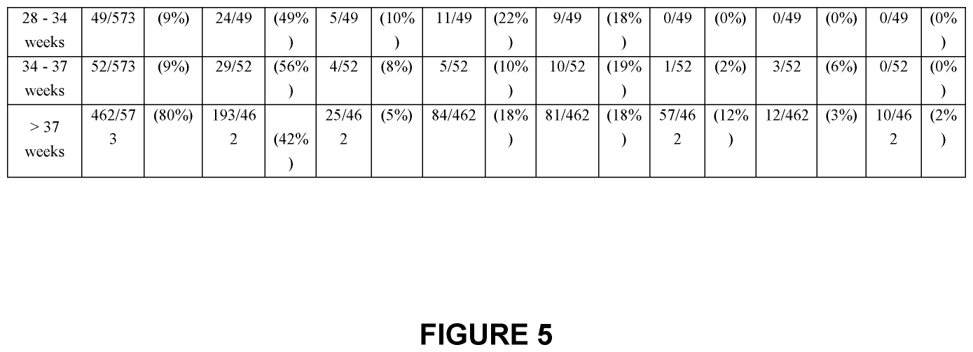

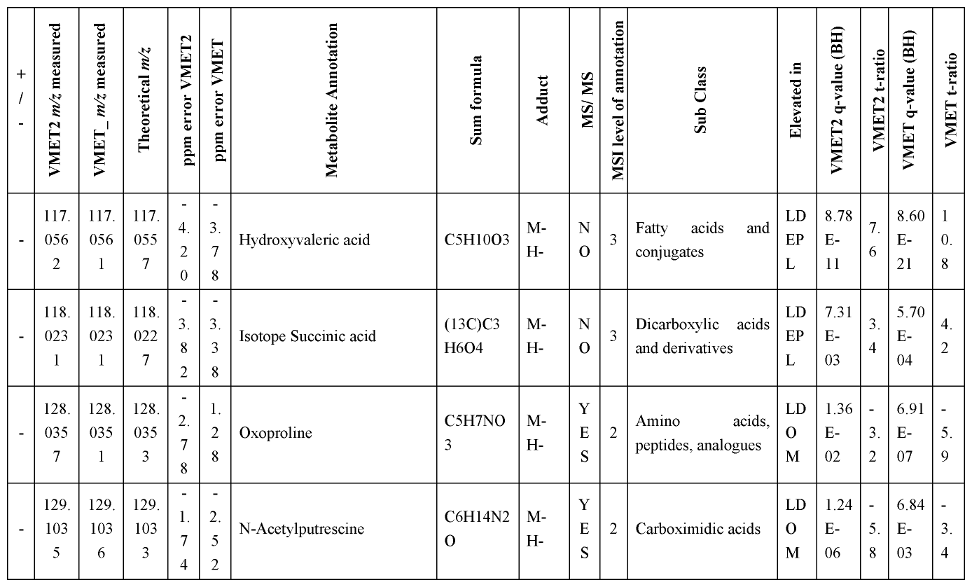

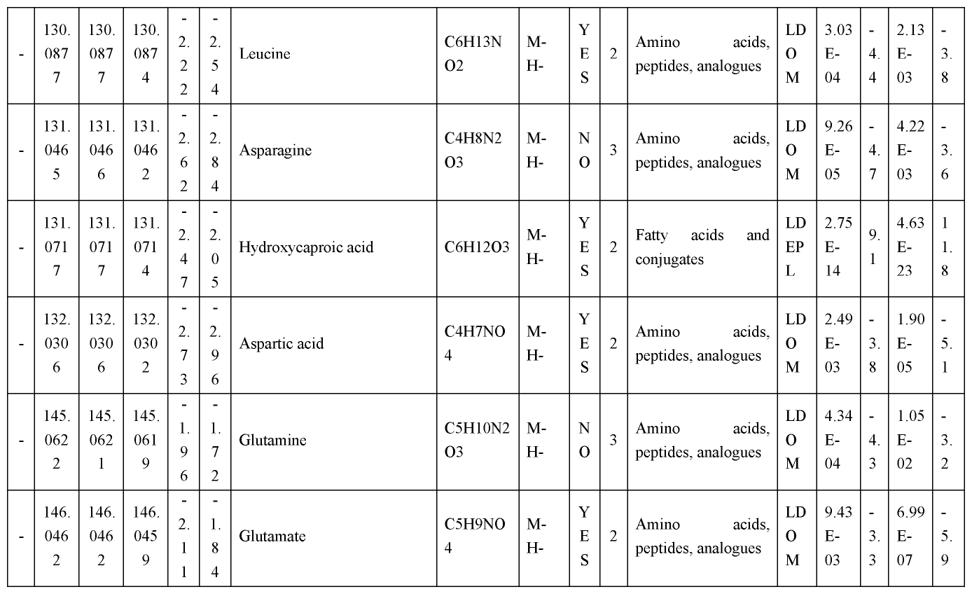

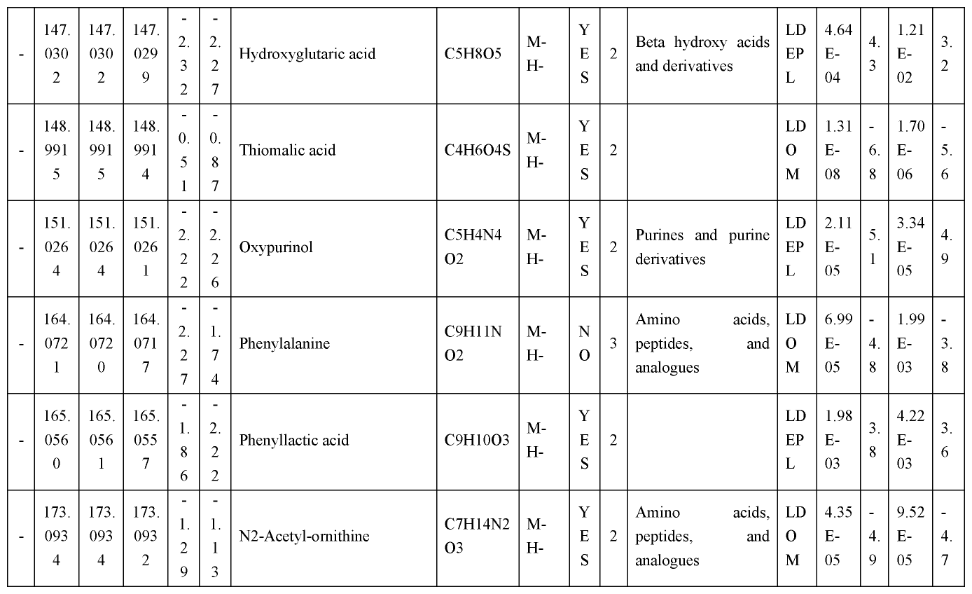

A total of six major CSTs were identified across both cohorts; 1. CST I (L. crispatus dominated); 2. CST II (L. gasseri dominated); 3. CST III (L. iners dominated); 4. CST IV (Gardnerella vaginalis dominated); 5. CST V (L. jensenii dominated); 6. CST VII (other Lactobacillus spp. dominated). A seventh group, dominated by Bifidobacterium breve (CST VI) was identified in the VMET2 cohort, but not in the VMET cohort, due to differences in the primer sets used for amplicon generation (see Methods and FIG. 3)^{16,41}. Higher taxonomic classification at genera level was achieved by grouping samples from CST I, II, III, V and VII into Lactobacillus-dominated (LDOM, VMET:n=379; VMET2:n=427), and samples from CST IV and VI into Lactobacillus-depleted (LDEPL, VMET:n=49; VMET2:n=112) categories (FIG. 5). Linear mixed effect models were applied on the longitudinal data sets to partition variance at each different time point across individuals, account for covariates (e.g. ethnicity, age, gestational age) and to derive metabolite signatures associated with individual vaginal microbiota compositions.

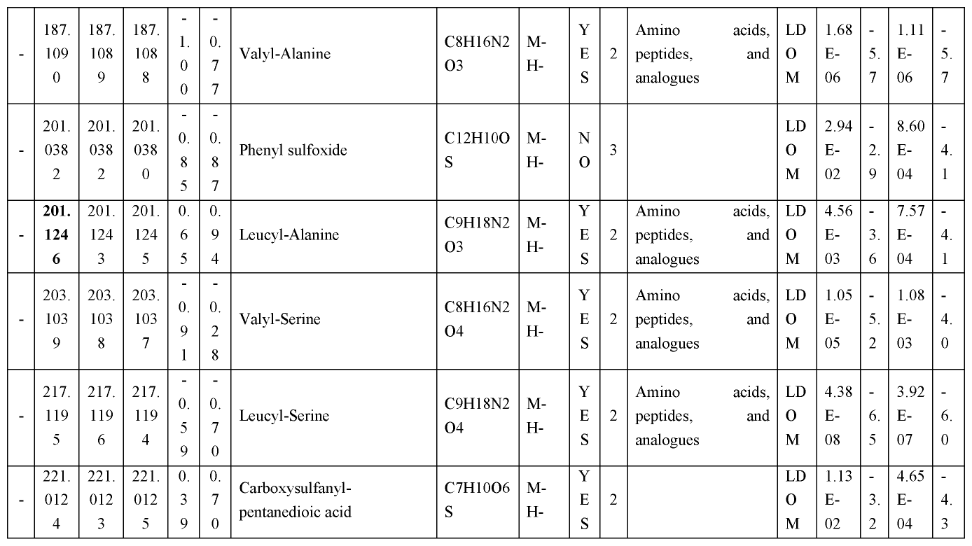

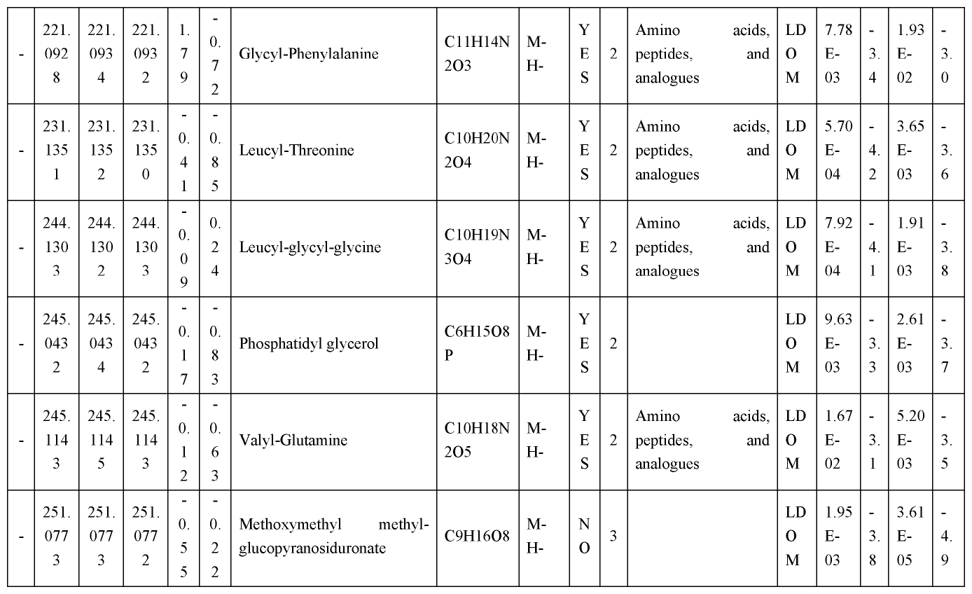

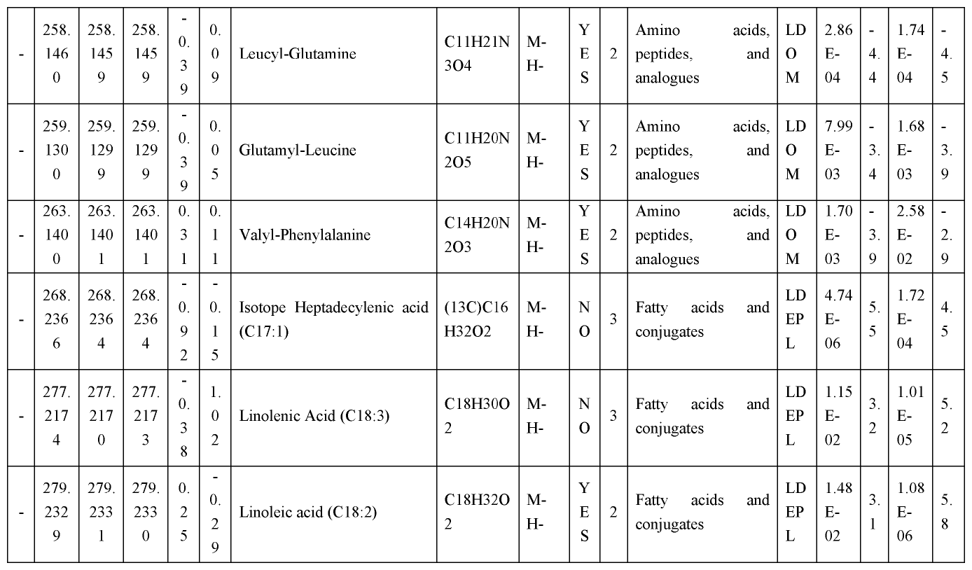

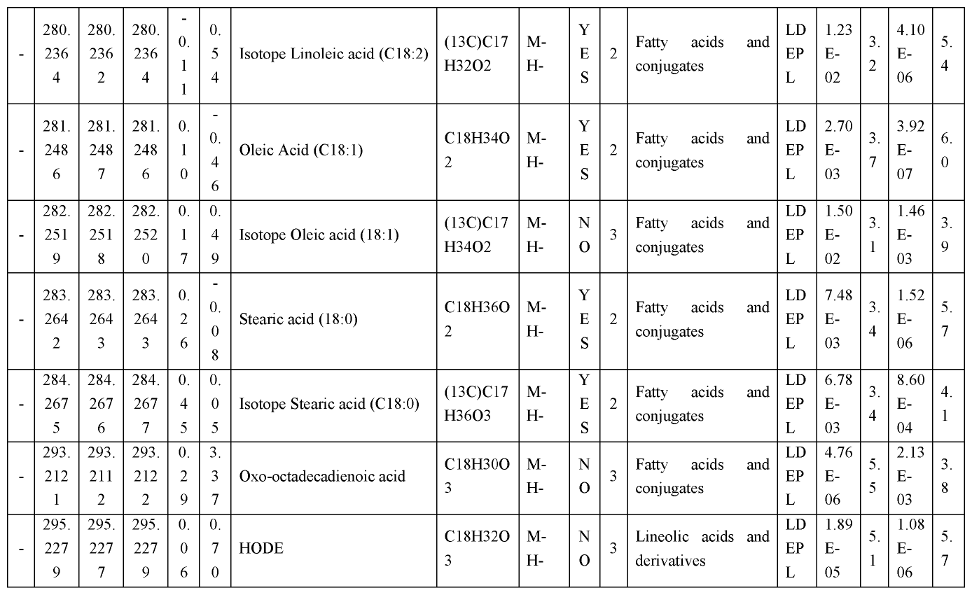

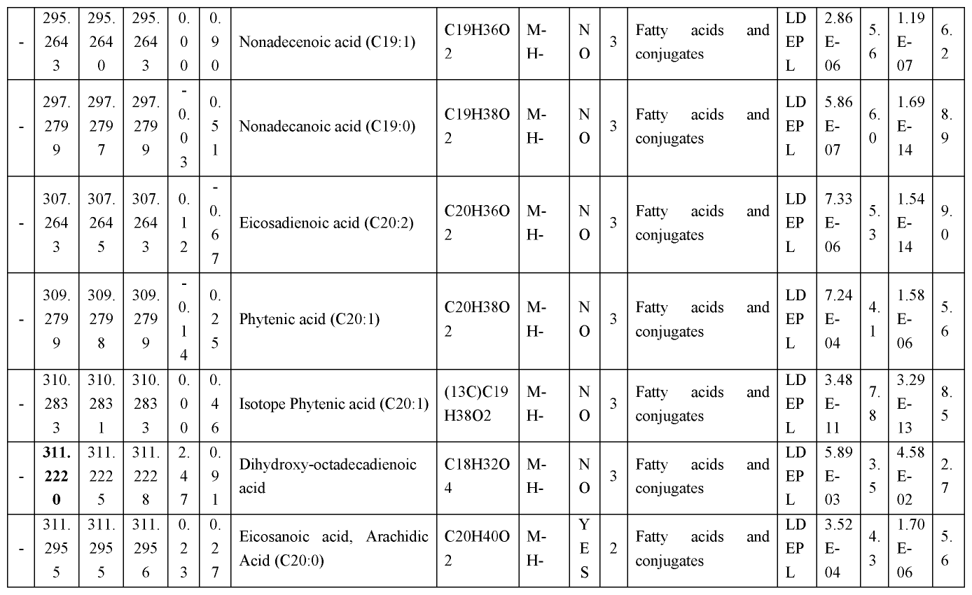

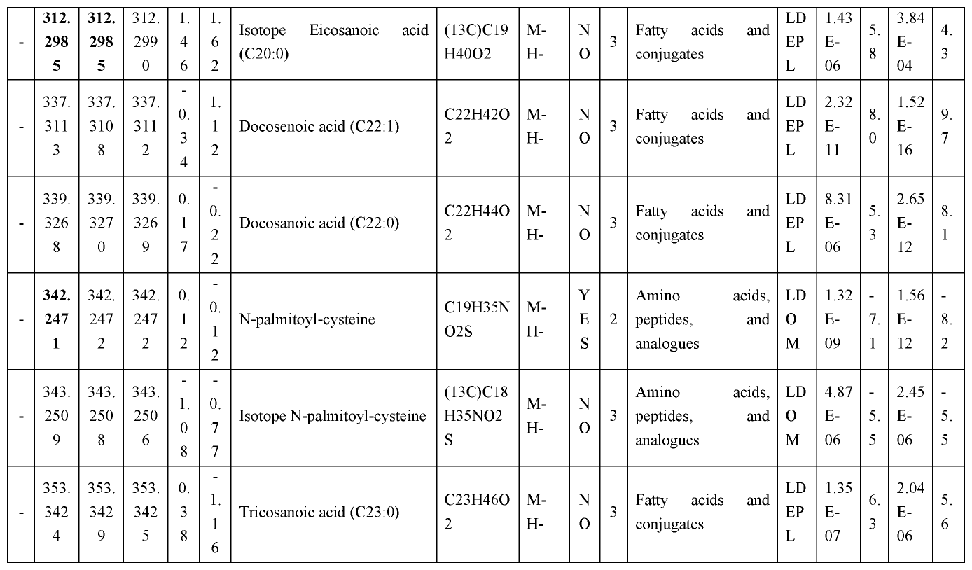

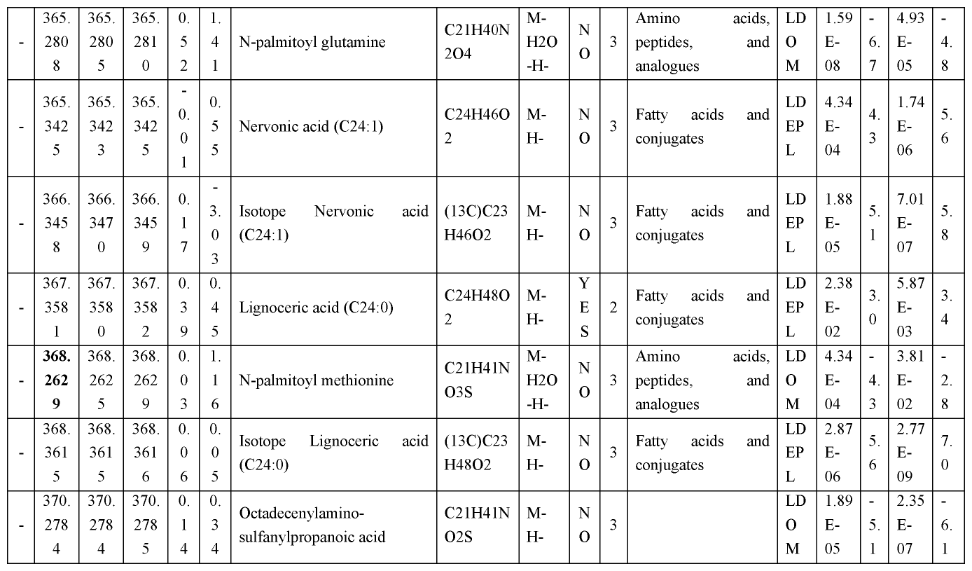

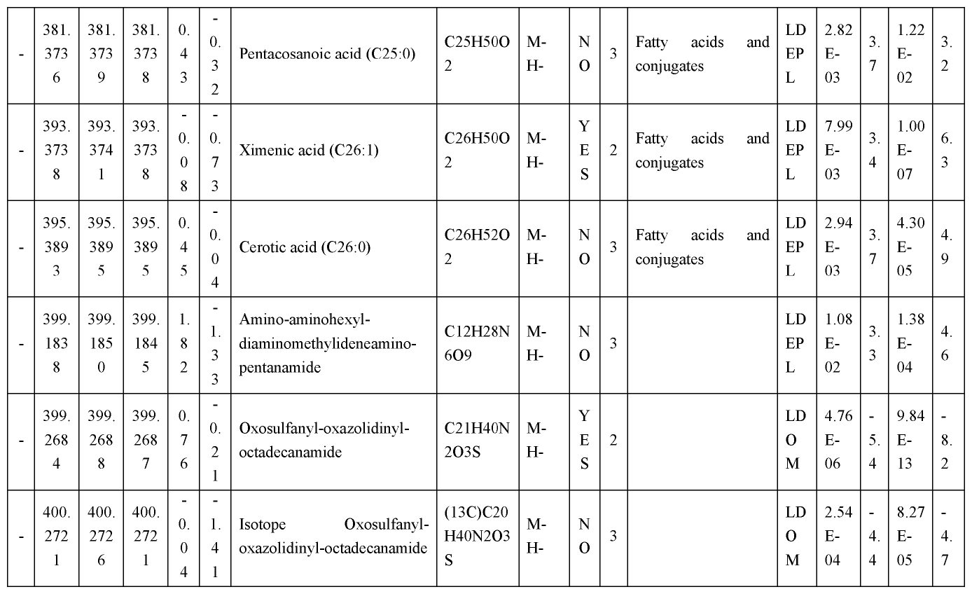

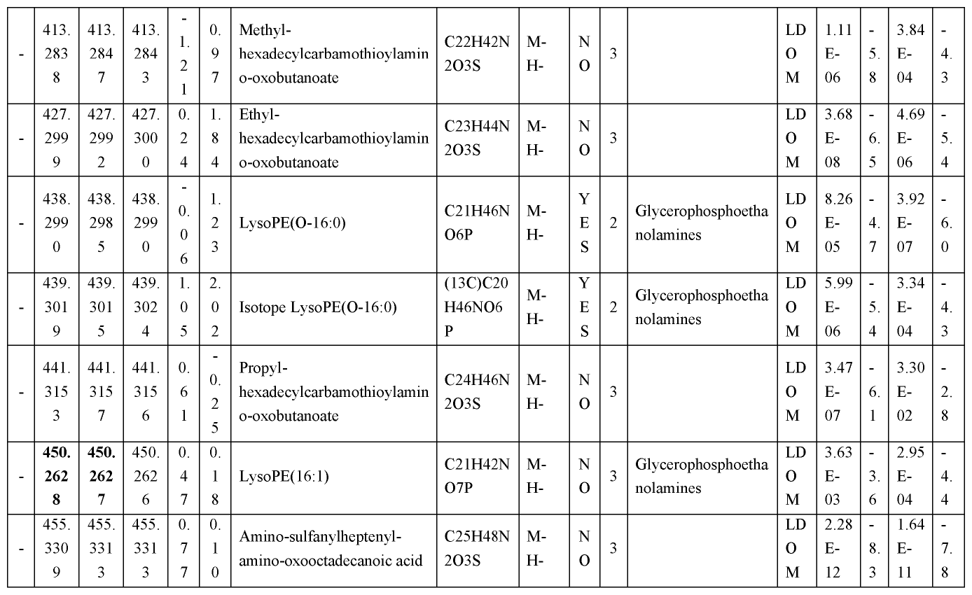

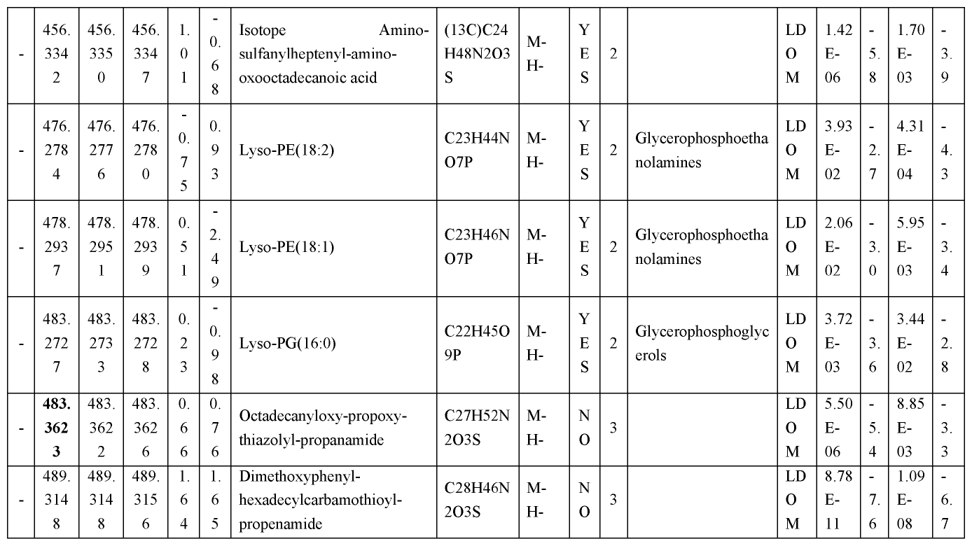

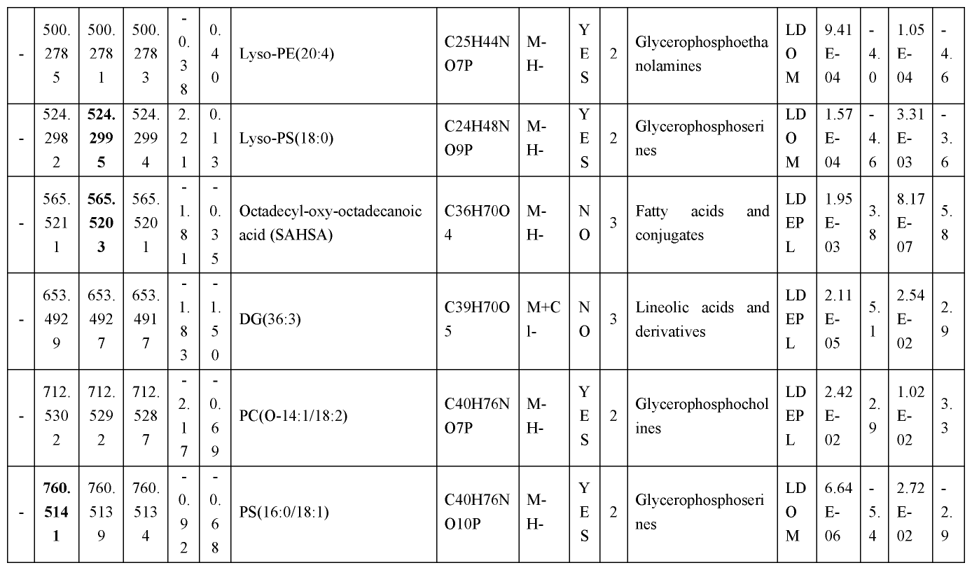

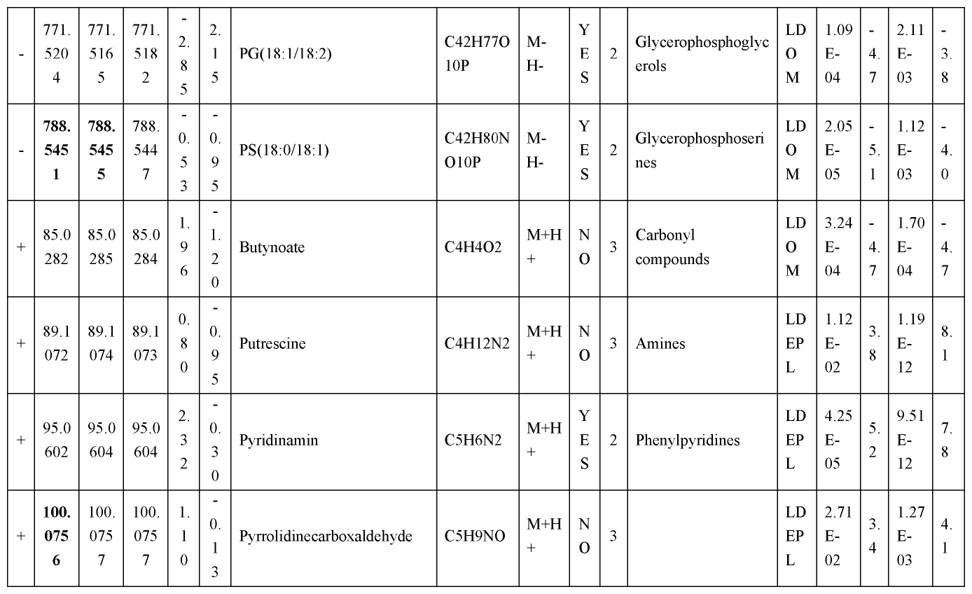

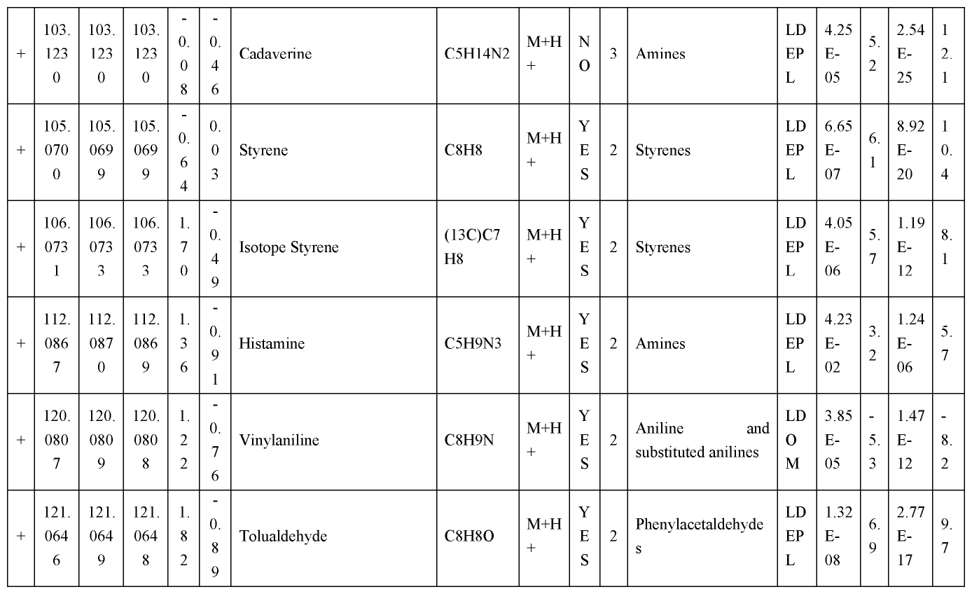

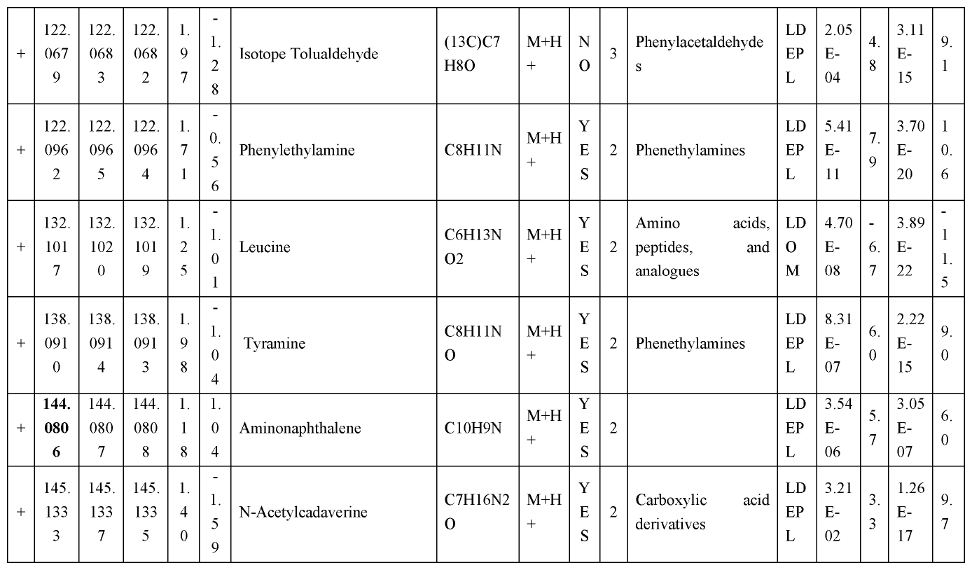

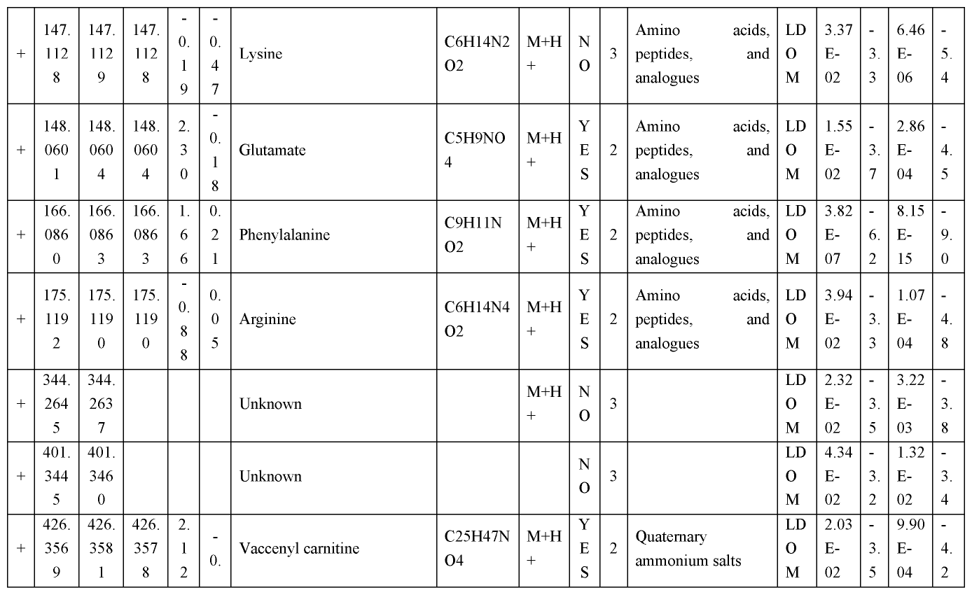

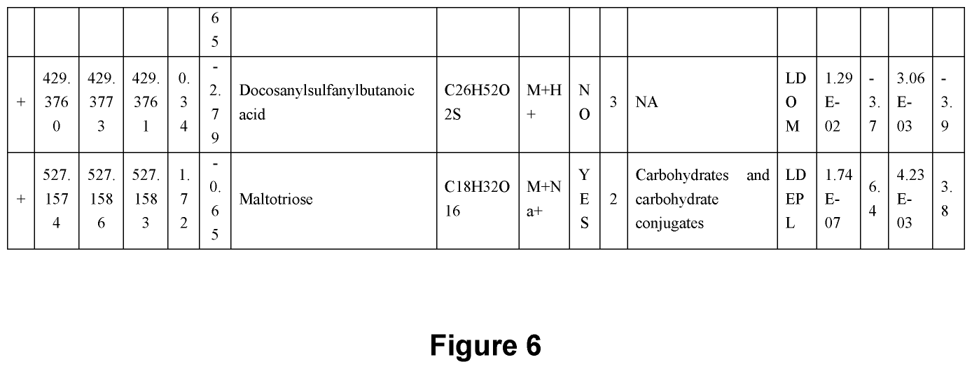

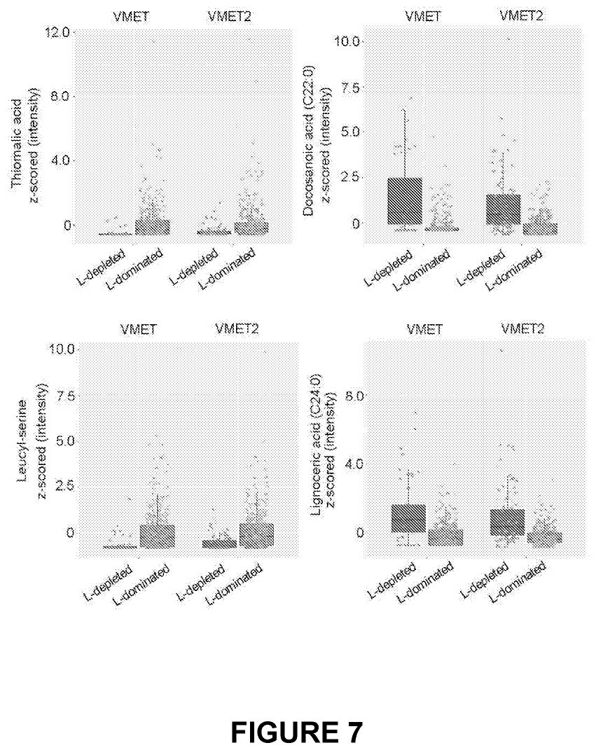

A total of 113 metabolite features detected in negative and positive ion modes were found to significantly discriminate between LDOM and LDEPL in both patient cohorts (FIG. 4) and included short and long chain fatty acids, amines, organic acids, carbohydrates, glycerophospholipids and sphingomyelins in the negative ion mode and alkaloids, benzenoids, carbohydrates, organic acids, fatty acyls, glycerophospholipids, sphingosines and steroids in the positive ion mode (FIG. 6). Four representative discriminatory metabolites are presented in FIG. 7 including thiomalic acid and leucyl-serine, which were both significantly higher in LDOM compositions, and docosanoic acid and lignoceric acid, which were significantly higher in LDEPL compositions.

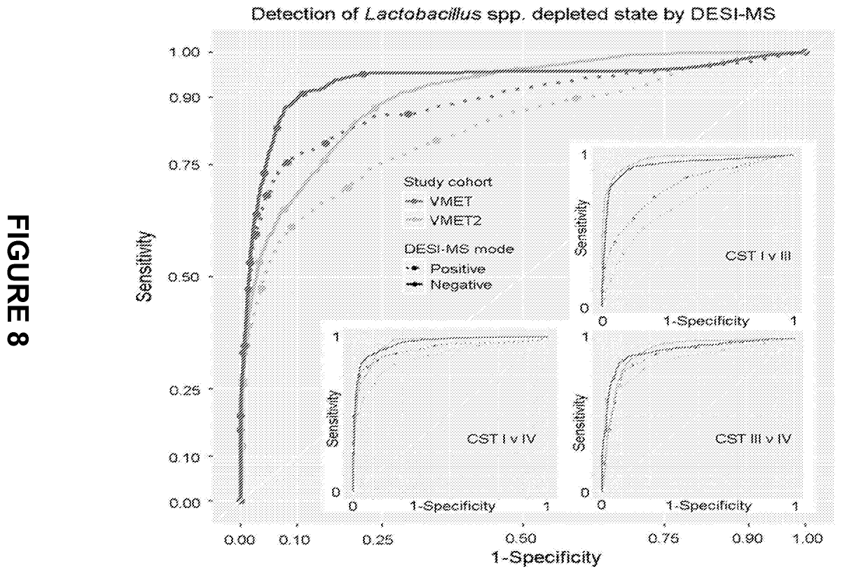

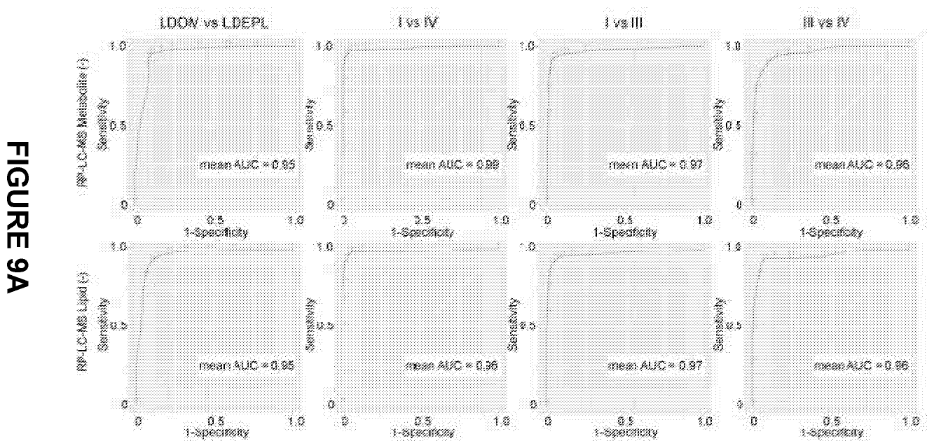

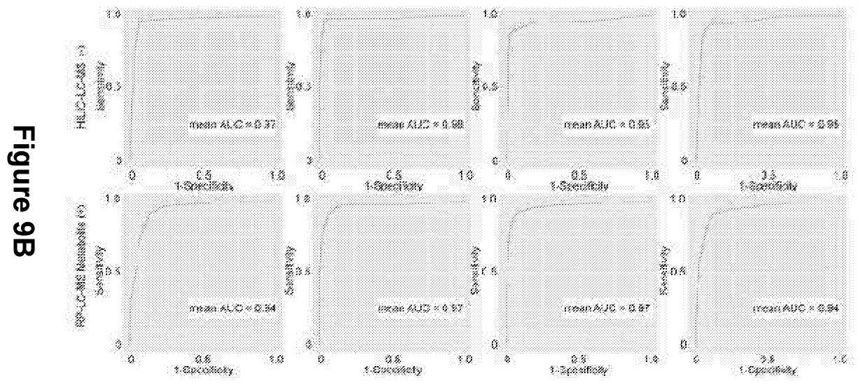

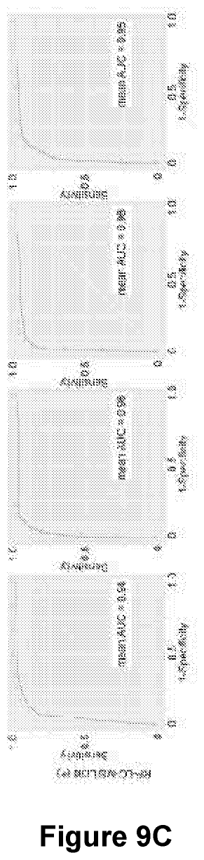

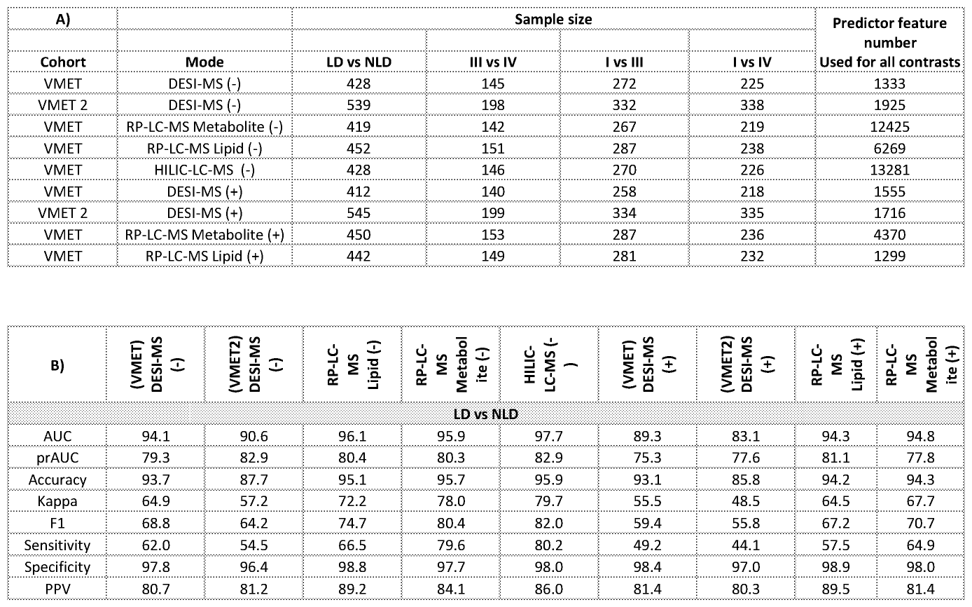

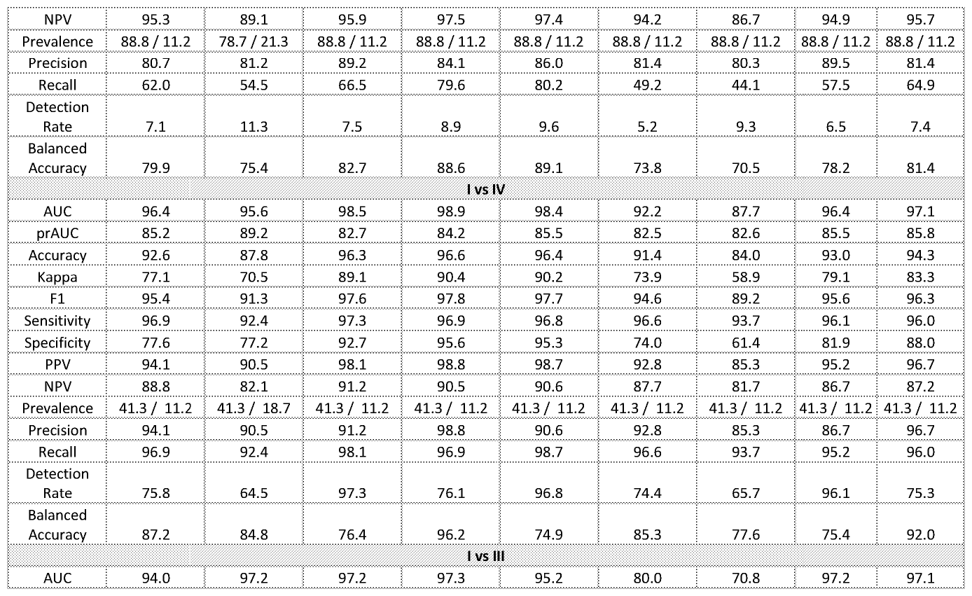

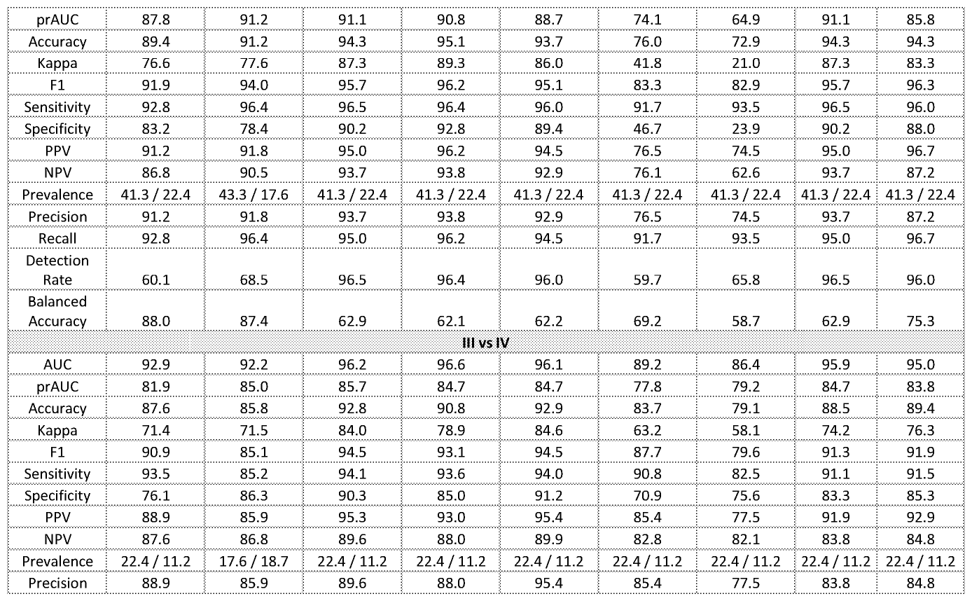

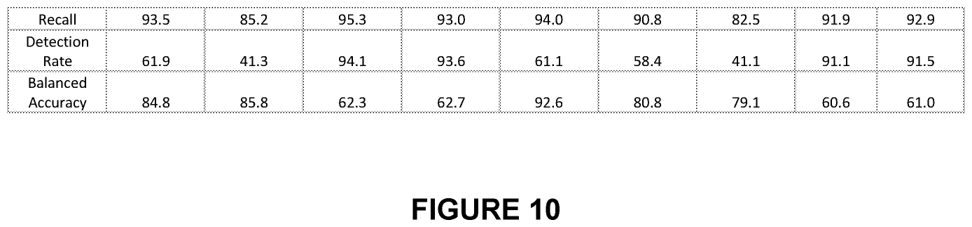

Using a random forest classifier and ROC-curve analysis, the performance of DESI-MS to predict vaginal microbiota composition was assessed in both cohorts using metabolic profiling data collected in both ionization modes (FIG. 8, FIG. 9). Robust predictive performance of genera-level classification (LDOM versus LDEPL) was observed across patient cohorts particularly using features derived from negative ion mode (VMET/VMET2; AUC 94.1/90.6, sensitivity: 62.0/54.5; specificity: 97.8/96.4) and was comparable when benchmarked against averaged (min/max) prediction performance of LC-MS assays (VMET/VMET2; AUC 95.76 (94.3-97.7), sensitivity:69.74 (57.5-80.2); specificity 98.28 (97.7-98.9); See FIG. 10).

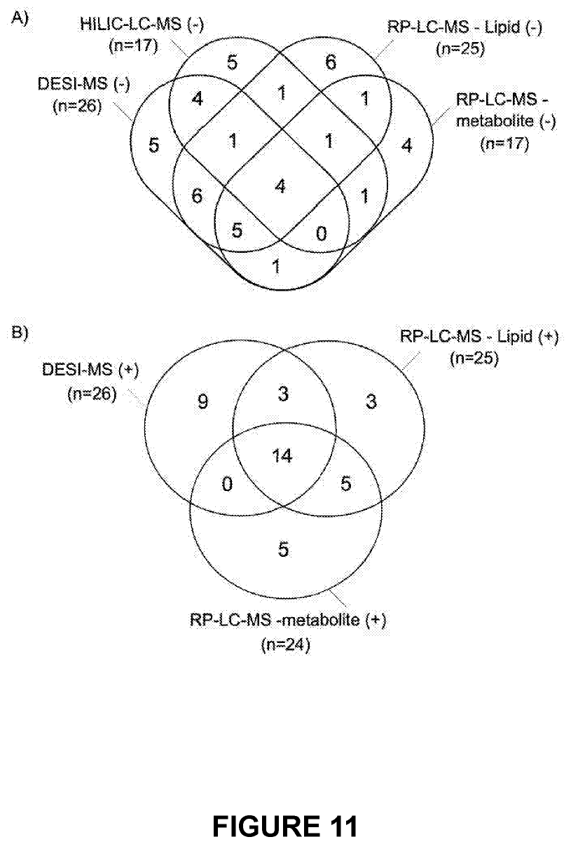

Inspection of the 16S rRNA amplicon sequencing data indicated that low sensitivity of prediction was largely associated with misclassification of samples with mixed compositional structures, often containing G. vaginalis. This is reflected in the representation of G. vaginalis in the HC analysis, which was distributed between three of the 11 original clusters. This suggests that hard-clustering techniques often used for community state typing may under-estimate the impact of low abundance taxa on the host mucosal metabolome. Comparison of predictive performances from the LC-MS assays indicated that metabolic coverage may also impact on misclassification rate with small polar or non-polar molecule based LC-MS assays marginally outperforming lipid-based assays (FIG. 11). Descrimination between the major vaginal CSTs (CSTI, III and IV) could also be readily achieved, with negative ion mode data providing the best performance (FIG. 8). Some descriminating features used in the predictive models were readily detected in culture swabs of Lactobacillus species (e.g. maltotriose) and taxa prevalent in CST IV communities (e.g. oxypurinol) analysed by DESI-MS.

### Example 2

### Assessment of Host Response at the Mucosal Interface using Direct On-Swab DESI-MS Profiling

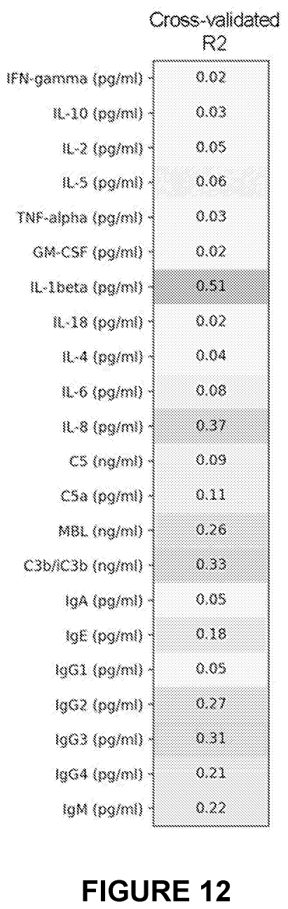

Activation of innate immunity in the lower reproductive tract often accompanies diseases states associated with suboptimal microbiota composition including BV, preterm birth, and sexually transmitted infections. Accordingly, the impact of the local vaginal immune response on the metabolic phenotype, and the potential of using direct on-swab metabolic profiling by DESI-MS to infer local inflammatory status was investigated. A panel of 22 soluble immune markers including cytokines, chemokines, immunoglobulins and members of the complement system were measured in a subset of matched cervicovaginal swab samples (n=391) from the VMET2 cohort. Random forest regression analysis was then used to predict the log-transformed concentrations of each marker using DESI-MS derived features. Robust prediction (cross-validated R²>0.25) was observed particularly for IL-1β (CV-R²=0.51), IL-8 (CV-R²=0.37), C3b/iC3b (CV-R²=0.31), IgG3 (CV-R²=0.31), IgG2 (CV-R²=0.27) and MBL (CV-R²=0.26) (FIG. 12).

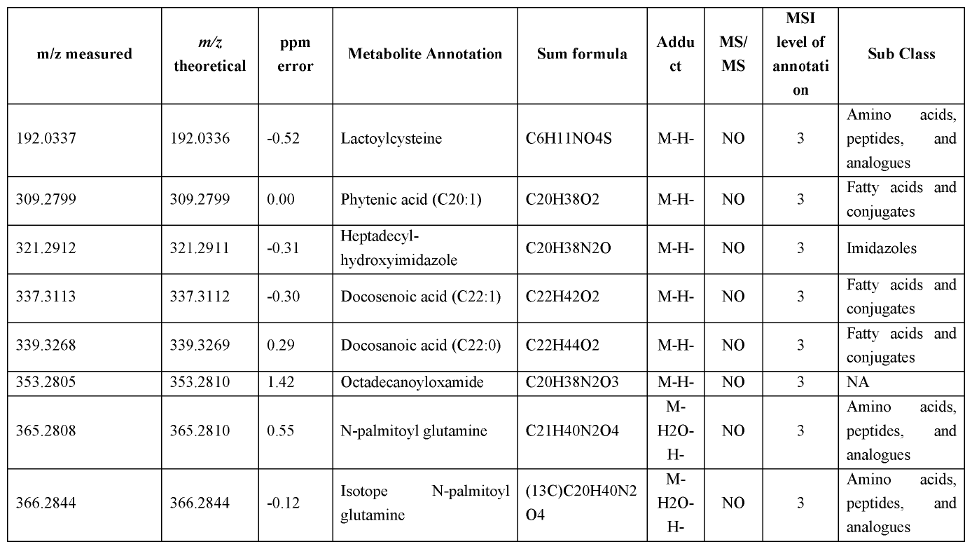

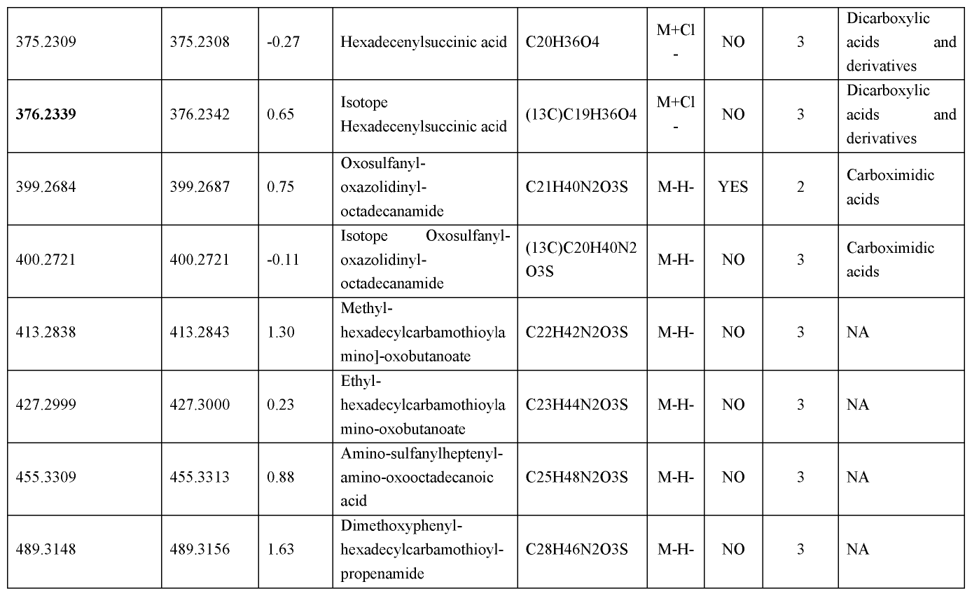

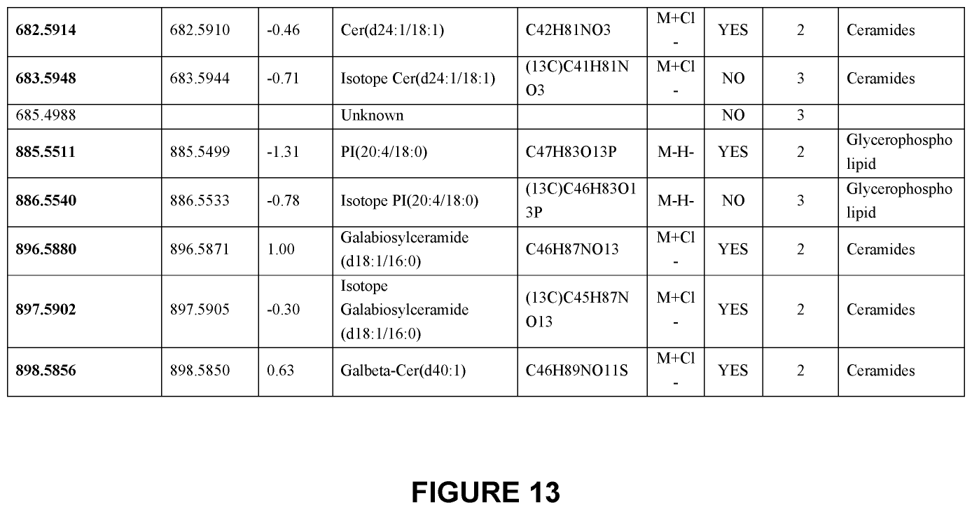

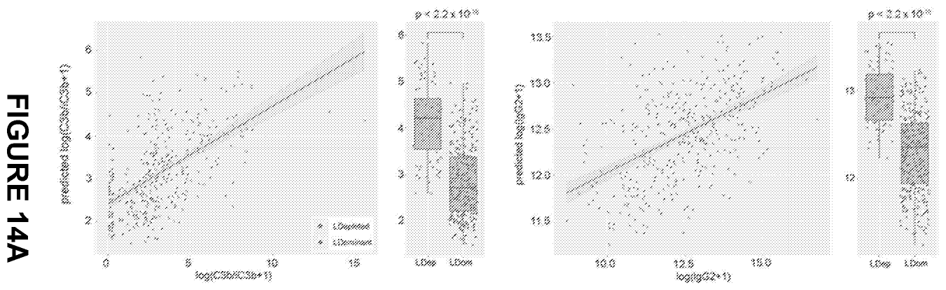

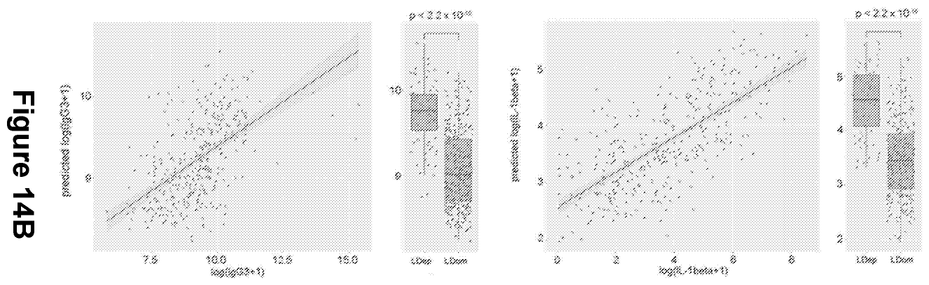

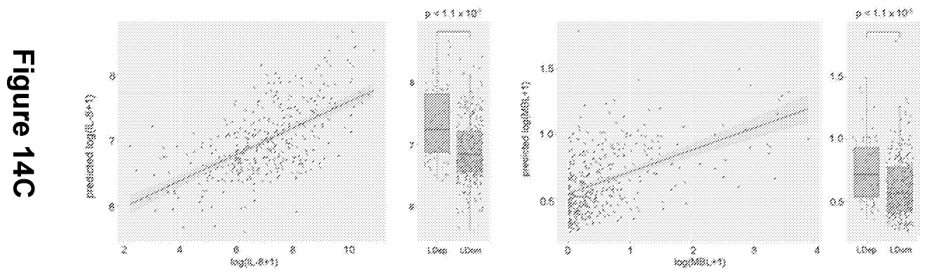

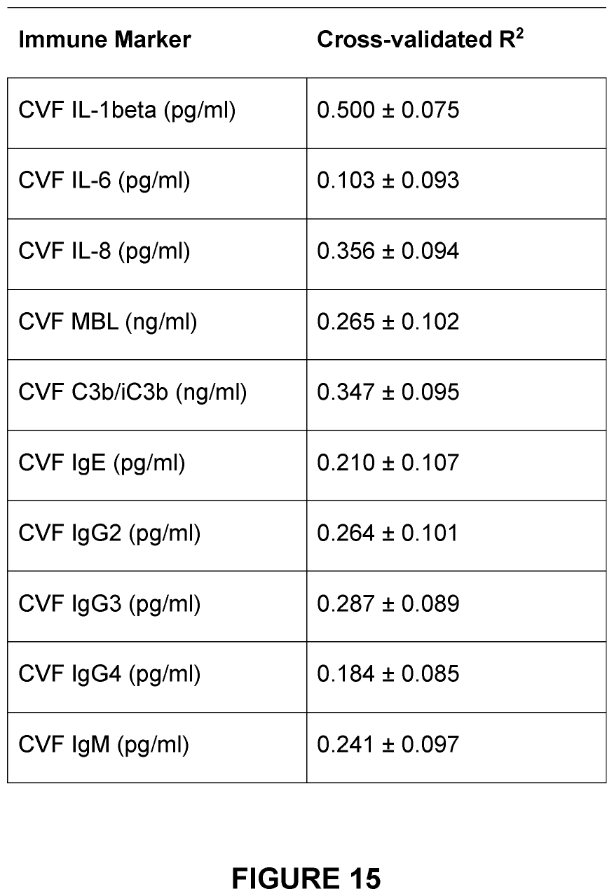

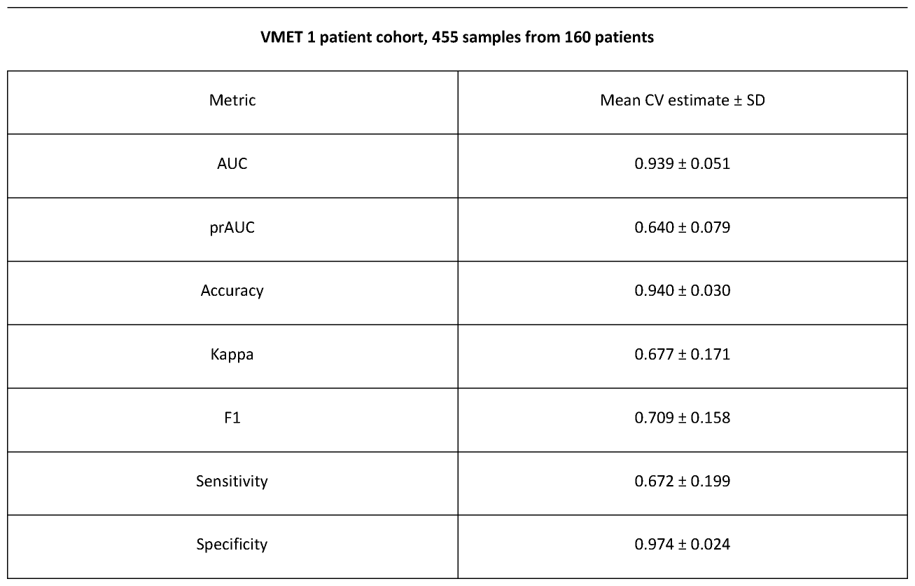

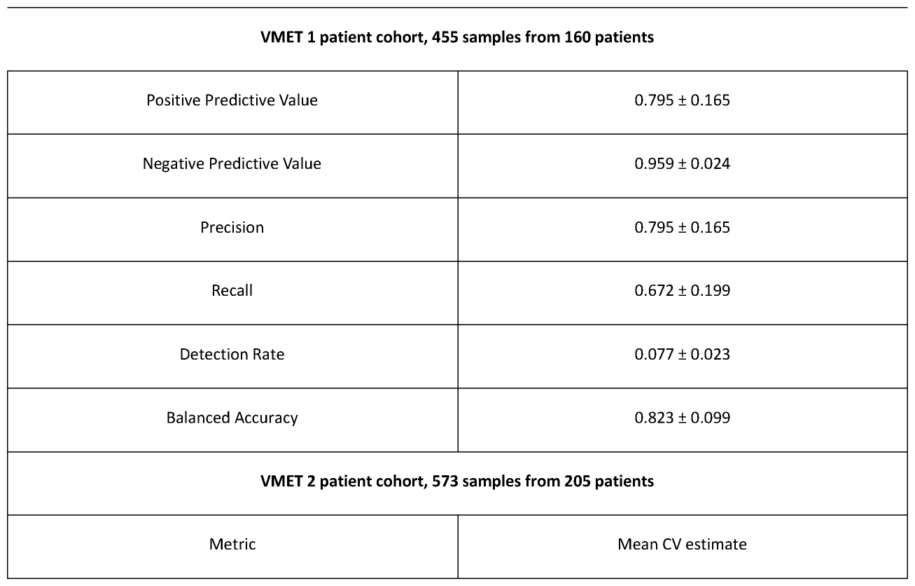

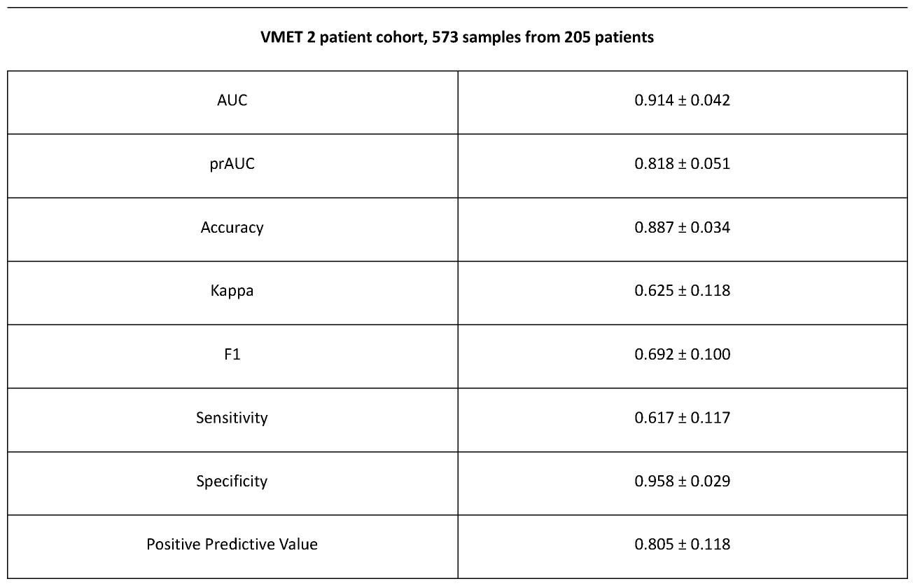

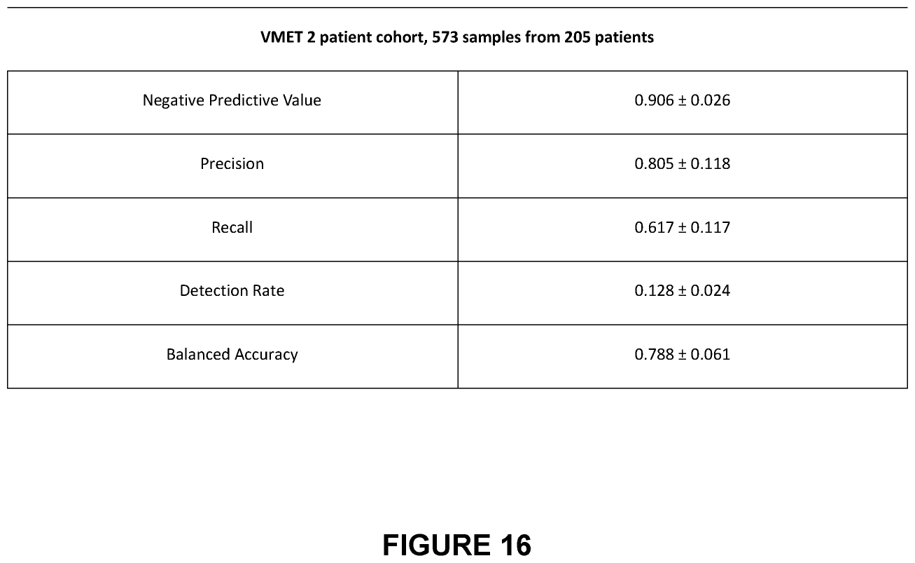

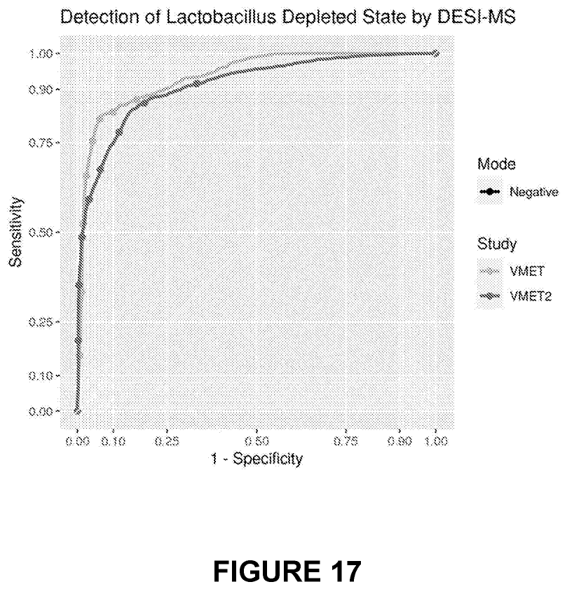

Plotting of the t-ratio for immune markers with CV-R²>0.1 (n=9) and DESI-MS features with R²>0.25 (n=23) revealed a metabolic signature strongly associated with local immune phenotype primarily characterized by altered levels of small secondary organic molecules such as amino acids, long chain fatty acids, glycerophospholipids and ceramides (FIG. 13). Both DESI-MS predicted and immunoassay-measured levels of C3b, pro-inflammatory IL-13, IgG2 and IgG3 were elevated in Lactobacillus depleted vaginal microbiomes indicating activation of local innate immune response (FIG. 14). However, many of the metabolites predictive of immune status were distinct from those predictive of microbial composition indicating their specificity to host response. A subset of 15 metabolites was found to be particularly useful in predicting inflammatory status/immune activation and an additional subset of 15 metabolites found to be particularly useful in predicting the microbial composition (FIGS. 15, 16 and 17).

### Example 3

### Vaginal Microbiome Instability and Immune Activation Associates with Preterm Birth Risk and Poor Outcomes Following Cervical Cerclage

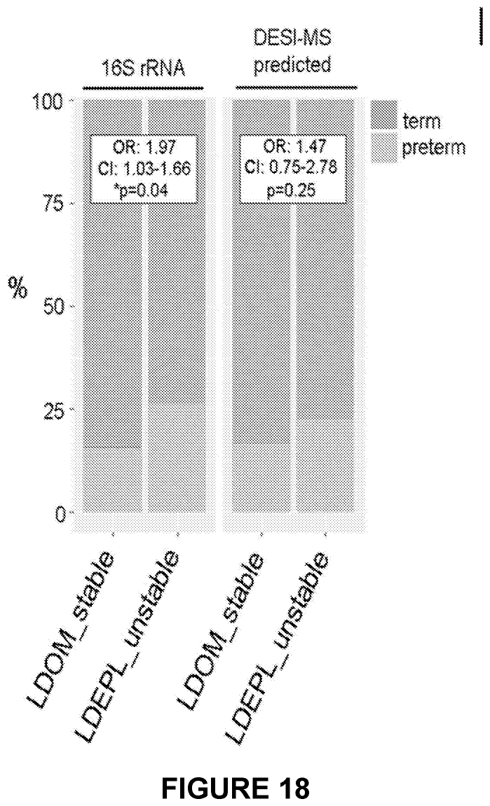

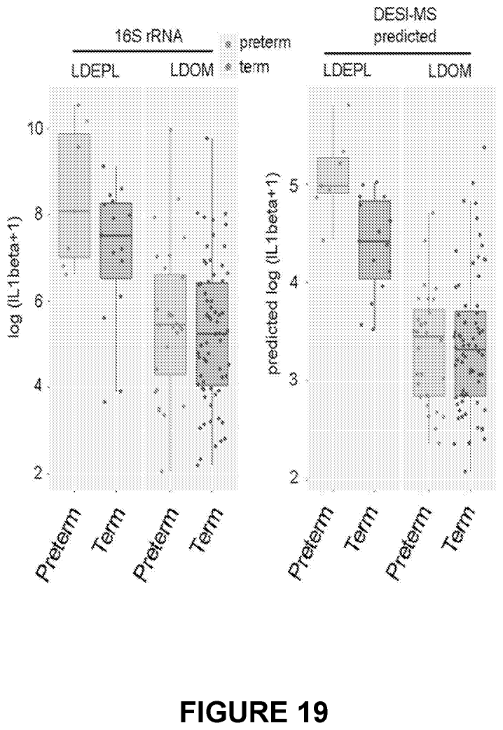

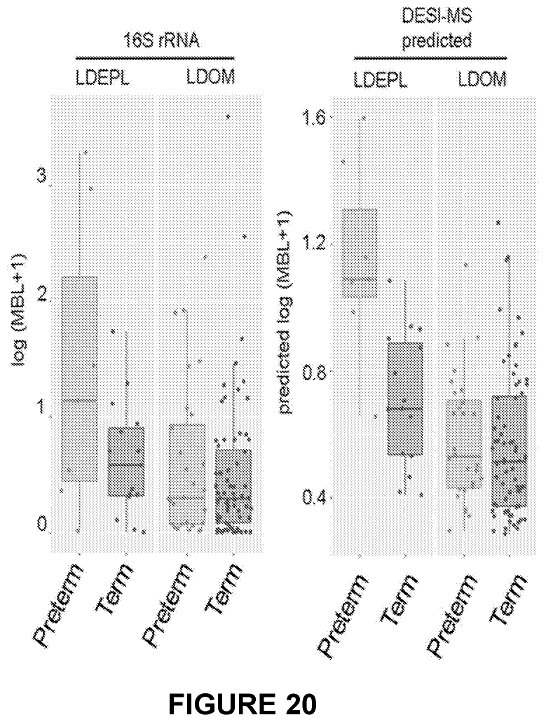

Subsequently, the relationship between key pregnancy outcomes and vaginal microbiome composition and inflammatory status as predicted by DESI-MS was investigated. High vaginal microbiome diversity and instability, as defined by shifts between Lactobacillus-dominated and Lactobacillus-depleted compositions, was associated with an increased risk of preterm birth compared to those women maintaining Lactobacillus-dominated compositions throughout pregnancy (16S rRNA-based metataxonomics; OR 1.97, CI 1.03-1.66, p=0.04; DESI-MS based prediction; OR 1.47, CI: 0.75-2.78, p=0.25) (FIG. 18). These results are in accordance with a recent meta-analysis reporting higher variance of vaginal bacterial communites across trimesters in women subsequently experiencing preterm birth (n=415 patients)⁴². Increased measured and DESI-MS predicted levels of IL-1β were associated with high-diversity vaginal microbiomes of both term and preterm birth outcome groups, but were highest in the latter (preterm 59.63 pg/ml versus term 1.94 pg/ml, p=0.006, Welch two sample t-test, FIG. 19). Increased levels of MBL were also observed in high-diversity vaginal microbiomes of women subsequently delivering preterm (term 0.042 ng/ml versus preterm 0.692 ng/ml, p=0.007, Welch two sample t-test, FIG. 20).

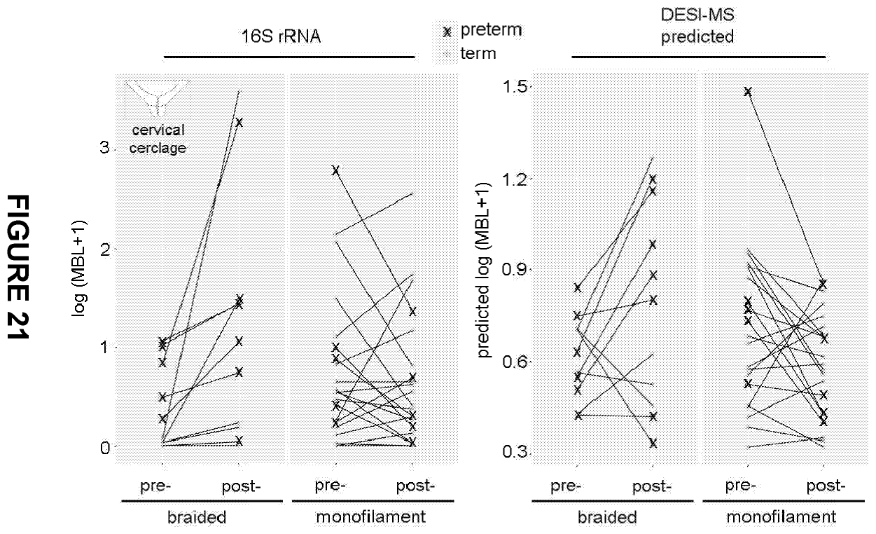

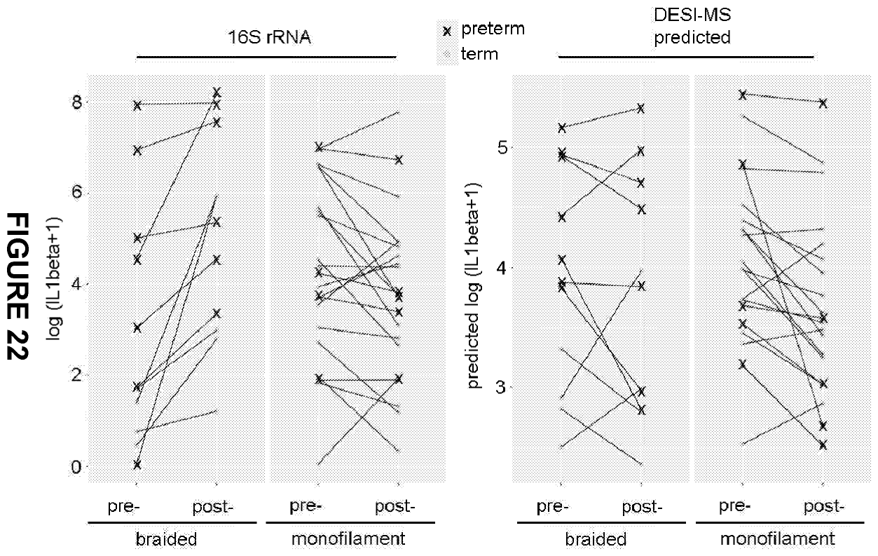

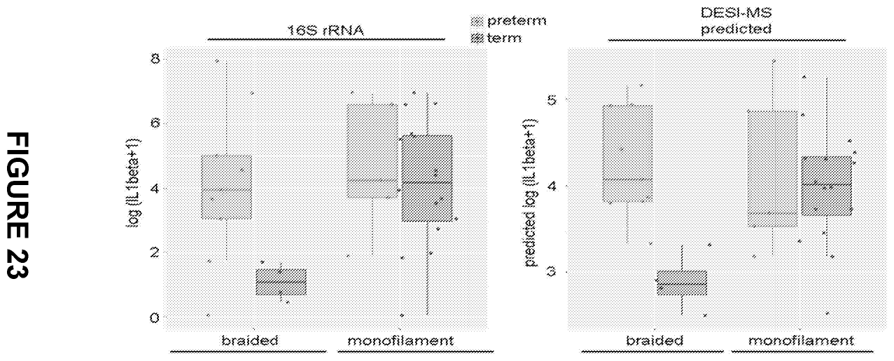

It is known that cervical cerclage, a procedure used to reinforce the cervical opening in women at risk of preterm delivery due to cervical shortening, if performed using a braided suture material, can induce vaginal bacterial dysbiosis, local inflammatory activation and premature cervical ripening associated with increased risk of preterm birth. Cerclage using a monofilament suture does not have these effects³⁶. Consistent with these findings, we observed increased cervicovaginal MBL levels in response to cervical cerclage using braided suture material, but rarely following the use of monofilament suture (10/11, 91% versus 9/21, 43%, p=0.011, Fisher's exact test). Similar results were observed when vaginal microbiome status and MBL levels were predicted using DESI-MS (7/11, 64% versus 8/21, 38%, p=0.316; FIG. 21). Immunoassay also indicated increased IL-1β post-braided cerclage but this was less consistently detected by DESI-MS prediction (FIG. 22). However, direct swab analysis by DESI-MS did accurately predict low levels of IL-1β in women delivering at term following treatment with braided cerclage compared to those who subsequently delivered preterm (FIG. 23). These results point toward an increased risk of preterm birth in those women receiving a braided cerclage who respond to it with an inflammatory phenotype.

Despite recent application of metataxonomic and metagenomic studies widening recognition of the role played by the vaginal microbiome in shaping women's health⁴³, examination of the vaginal microbiome in clinical settings remains limited to culture and microscopy, which like molecular-based methods, fail to capture clinically relevant information regarding host response. The method herein disclosed, which is easily amenable to bedside point of care testing, addresses this by rapidly leveraging microbial and host derived information contained within the metabolic milieu of the mucosal interface, to provide robust detection of key vaginal bacterial compositions and simultaneous estimation of host immune response. Such information is desirable for informing treatment strategy in an increasing number of clinical scenarios. For example, efficacy of the topical pre-exposure prophylactic, Tenofovir, is superior in preventing HIV acquisition in women with Lactobacillus-dominated vaginal microbiomes compared to those with Lactobacillus depletion and increased relative abundance of G. vaginalis and BV-associated bacteria⁴⁴.

Many of the discriminatory metabolites used in the statistical models herein disclosed are consistent with those previously identified using GC-MS or LC-MS based assays in women suffering from BV^{45,46,47,48} and have biologically plausible roles in mediating vaginal health and disease. For example, most of the observed long-chain fatty acids identified have been associated with cellular stress or inflammatory processes⁴⁹. Increased vaginal levels of SCFAs associate with activation of pro-inflammatory pathways^{50,51,52,53} that contribute to reduced barrier integrity of the vaginal epithelium and consequently increased risk of infection⁵⁴. Similarly, elevated vaginal biogenic amines reported in BV are associated with lipid metabolism and activation of innate immune response^{46,55,56}. An additional strength of the approach herein disclosed is the ability to simultaneously capture aspects of host response, which broadly reflect recognized relationships between vaginal microbiome composition and innate immune response. For example, L. crispatus-dominance of the vaginal niche is associated with suppressed levels of pro-inflammatory IL-1β compared to high-diversity compositions or L. iners dominance⁵⁷⁻⁶². MBL is a recognition molecule for G. vaginalis⁶³ and several single nucleotide polymorphisms in the MBL2 gene have been reported to increase the risk of BV^{64,65}.

Accordingly, the results herein disclosed support that DESI-MS captures sufficient information to allow genus and species level classification directly from mucosal samples, for example, vaginal swabs, without the requirement of sample preparation and within a fraction of the time.

### REFERENCES

1. Witkin, S. S., Linhares, I. M. & Giraldo, P. Bacterial flora of the female genital tract: function and immune regulation. Best practice & research. Clinical obstetrics & gynaecology 21, 347-354 (2007).
2. Jespers, V., et al. A longitudinal analysis of the vaginal microbiota and vaginal immune mediators in women from sub-Saharan Africa. Sci Rep 7, 11974 (2017).
3. Campisciano, G., Zanotta, N., Licastro, D., De Seta, F. & Comar, M. In vivo microbiome and associated immune markers: New insights into the pathogenesis of vaginal dysbiosis. Sci Rep 8, 2307 (2018).
4. Pruski, P., et al. Medical Swab Analysis Using Desorption Electrospray Ionization Mass Spectrometry: A Noninvasive Approach for Mucosal Diagnostics. Anal Chem 89, 1540-1550 (2017).
5. Izzi-Engbeaya, C., et al. The effects of kisspeptin on beta-cell function, serum metabolites and appetite in humans. Diabetes Obes Metab 20, 2800-2810 (2018).
6. Lewis, M. R., et al. Development and Application of Ultra-Performance Liquid Chromatography-TOF MS for Precision Large Scale Urinary Metabolic Phenotyping. Anal Chem 88, 9004-9013 (2016).
7. Kessner, D., Chambers, M., Burke, R., Agus, D. & Mallick, P. ProteoWizard: open source software for rapid proteomics tools development. Bioinformatics (Oxford, England) 24, 2534-2536 (2008).
8. Gibb, S. & Strimmer, K. MALDIquant: a versatile R package for the analysis of mass spectrometry data. Bioinformatics (Oxford, England) 28, 2270-2271 (2012).
9. Smith, C. A., Want, E. J., O'Maille, G., Abagyan, R. & Siuzdak, G. XCMS: processing mass spectrometry data for metabolite profiling using nonlinear peak alignment, matching, and identification. Anal Chem 78, 779-787 (2006).
10. Sands, C. J., et al. The nPYc-Toolbox, a Python module for the pre-processing, quality-control and analysis of metabolic profiling datasets. Bioinformatics (Oxford, England) 35, 5359-5360 (2019).
11. Smith, C. A., et al. METLIN: a metabolite mass spectral database. Ther Drug Monit 27, 747-751 (2005).
12. Fahy, E. A comprehensive classification system for lipids (vol 46, pg 839, 2005). Journal of Lipid Research 51, 1618-1618 (2010).
13. Wishart, D. S., et al. HMDB 3.0—The Human Metabolome Database in 2013. Nucleic Acids Res 41, D801-807 (2013).
14. Sumner, L. W., et al. Proposed minimum reporting standards for chemical analysis Chemical Analysis Working Group (CAWG) Metabolomics Standards Initiative (MSI). Metabolomics 3, 211-221 (2007).
15. Macintyre, D. A., et al. The vaginal microbiome during pregnancy and the postpartum period in a European population. Sci Rep 5, 8988 (2015).
16. Frank, J. A., et al. Critical evaluation of two primers commonly used for amplification of bacterial 16S rRNA genes. Appl Environ Microbiol 74, 2461-2470 (2008).
17. Schloss, P. D., et al. Introducing mothur: Open-Source, Platform-Independent, Community-Supported Software for Describing and Comparing Microbial Communities. Applied and Environmental Microbiology 75, 7537-7541 (2009).
18. Wang, Q., Garrity, G. M., Tiedje, J. M. & Cole, J. R. Naïve Bayesian Classifier for Rapid Assignment of rRNA Sequences into the New Bacterial Taxonomy. Applied and Environmental Microbiology 73, 5261 (2007).
19. Edgar, R. C. Search and clustering orders of magnitude faster than BLAST. Bioinformatics (Oxford, England) 26, 2460-2461 (2010).
20. Fettweis, J. M., et al. Species-level classification of the vaginal microbiome. BMC Genomics 13 Suppl 8, S17 (2012).
21. Virtanen, P., et al. SciPy 1.0: fundamental algorithms for scientific computing in Python. Nature methods 17, 261-272 (2020).
22. Hunter, J. D. Matplotlib: A 2D Graphics Environment. Computing in Science & Engineering 9, 90-95 (2007).
23. Waskom, M., et al. mwaskom/seaborn: v0.11.0. (2020).
24. Schulthess, B., et al. Identification of Gram-positive cocci by use of matrix-assisted laser desorption ionization-time of flight mass spectrometry: comparison of different preparation methods and implementation of a practical algorithm for routine diagnostics. J Clin Microbiol 51, 1834-1840 (2013).
25. Bates, D., Mächler, M., Bolker, B. & Walker, S. Fitting Linear Mixed-Effects Models Using Ime4. Journal of Statistical Software 67, 1-48 (2015).
26. Lenth, R., et al. Estimated Marginal Means, aka Least-Squares Means (v1.5.2-1). https://cran.r-project.org/web/packages/emmeans/(2020).
27. Halekoh, U. & Højsgaard, S. A Kenward-Roger Approximation and Parametric Bootstrap Methods for Tests in Linear Mixed Models The R Package pbkrtest. Journal of Statistical Software 59 (2012).
28. Liaw, A. & Wiener, M. randomForest:Breiman and Cutler's Random Forests for Classification and Regression. https://cran.r-project.org/web/packages/randomForest/index.html (2018).
29. Kuhn, M. caret: Classification and Regression Training (v6.0-86). https://CRAN.R-project.org/package=caret (2020).
30. Robin, X., et al. pROC:an open-source package for R and S+ to analyze and compare ROC curves. BMC Bioinformatics 12, 77 (2011).
31. Sachs, M. C. plotROC: A Tool for Plotting ROC Curves. J Stat Softw 79 (2017).
32. Saito, T. & Rehmsmeier, M. Precrec: fast and accurate precision-recall and ROC curve calculations in R. Bioinformatics (Oxford, England) 33, 145-147 (2017).
33. Wickham, H. ggplot2:Elegant Graphics for Data Analysis, (Springer-Verlag, New York, 2016).
34. Al-Memar, M., et al. The association between vaginal bacterial composition and miscarriage:a nested case-control study. BJOG 127, 264-274 (2020).
35. Kindinger, L. M., et al. The interaction between vaginal microbiota, cervical length, and vaginal progesterone treatment for preterm birth risk. Microbiome 5, 6 (2017).
36. Kindinger, L. M., et al. Relationship between vaginal microbial dysbiosis, inflammation, and pregnancy outcomes in cervical cerclage. Science translational medicine 8, 350ra102 (2016).
37. Brown, R. G., et al. Vaginal dysbiosis increases risk of preterm fetal membrane rupture, neonatal sepsis and is exacerbated by erythromycin. BMC Med 16, 9 (2018).
38. Fettweis, J. M., et al. The vaginal microbiome and preterm birth. Nature Medicine 25, 1012-1021 (2019).
39. Ravel, J., et al. Vaginal microbiome of reproductive-age women. Proc Natl Acad Sci U S A 108 Suppl 1, 4680-4687 (2011).
40. DiGiulio, D. B., et al. Temporal and spatial variation of the human microbiota during pregnancy. Proc Natl Acad Sci U S A 112, 11060-11065 (2015).
41. Brown, R. G., et al. Prospective observational study of vaginal microbiota pre-and post-rescue cervical cerclage. BJOG 126, 916-925 (2019).
42. Kosti, I., Lyalina, S., Pollard, K. S., Butte, A. J. & Sirota, M. Meta-Analysis of Vaginal Microbiome Data Provides New Insights Into Preterm Birth. Front Microbiol 11, 476 (2020).
43. van de Wijgert, J. H., et al. The vaginal microbiota:what have we learned after a decade of molecular characterization? PloS one 9, e105998 (2014).
44. Klatt, N. R., et al. Vaginal bacteria modify HIV tenofovir microbicide efficacy in African women. Science 356, 938-945 (2017).
45. McMillan, A., et al. A multi-platform metabolomics approach identifies highly specific biomarkers of Jones, B. M. Inhibition of chemotaxis by organic acids from anaerobes may prevent a purulent response in bacterial vaginosis. J Med Microbiol 49, 1023-1030 (2000). bacterial diversity in the vagina of pregnant and non-pregnant women. Sci Rep 5, 14174 (2015).
46. Srinivasan, S., et al. Metabolic signatures of bacterial vaginosis. MBio 6 (2015).
47. Vitali, B., et al. Vaginal microbiome and metabolome highlight specific signatures of bacterial vaginosis. Eur J Clin Microbiol Infect Dis 34, 2367-2376 (2015).
48. Nelson, T. M., et al. Vaginal biogenic amines: biomarkers of bacterial vaginosis or precursors to vaginal dysbiosis? Front. Physiol. 6, 253 (2015).
49. Rutkowsky, J. M., et al. Acylcarnitines activate proinflammatory signaling pathways. Am J Physiol Endocrinol Metab 306, E1378-1387 (2014).
50. Al-Mushrif, S., Eley, A. & Jones, B. M. Inhibition of chemotaxis by organic acids from anaerobes may prevent a purulent response in bacterial vaginosis. J Med Microbiol 49, 1023-1030 (2000).
51. Aldunate, M., et al. Vaginal concentrations of lactic acid potently inactivate HIV. J Antimicrob Chemother 68, 2015-2025 (2013).
52. Mirmonsef, P., et al. Short-chain fatty acids induce pro-inflammatory cytokine production alone and in combination with toll-like receptor ligands. Am J Reprod Immunol 67, 391-400 (2012).
53. O'Hanlon, D. E., Moench, T. R. & Cone, R. A. In vaginal fluid, bacteria associated with bacterial vaginosis can be suppressed with lactic acid but not hydrogen peroxide. BMC Infect Dis 11, 200 (2011).
54. Nazli, A., et al. Exposure to HIV-1 directly impairs mucosal epithelial barrier integrity allowing microbial translocation. PLOS Pathog 6, e1000852 (2010).
55. Wolrath, H., Boren, H., Hallen, A. & Forsum, U. Tirimethylamine content in vaginal secretion and its relation to bacterial vaginosis. Apmis 110, 819-824 (2002).
56. Yeoman, C. J., et al. A multi-omic systems-based approach reveals metabolic markers of bacterial vaginosis and insight into the disease. PloS one 8, e56111 (2013).
57. Anahtar, M. N., et al. Cervicovaginal bacteria are a major modulator of host inflammatory responses in the female genital tract. Immunity 42, 965-976 (2015).
58. Hedges, S. R., Barrientes, F., Desmond, R. A. & Schwebke, J. R. Local and systemic cytokine levels in relation to changes in vaginal flora. J Infect Dis 193, 556-562 (2006).
59. Mattsby-Baltzer, I., Platz-Christensen, J. J., Hosseini, N. & Rosen, P. IL-1 beta, IL-6, TNF alpha, fetal fibronectin, and endotoxin in the lower genital tract of pregnant women with bacterial vaginosis. Acta Obstet Gyn Scan 77, 701-706 (1998).
60. Platz-Christensen, J. J., Mattsby-Baltzer, I., Thomsen, P. & Wiqvist, N. Endotoxin and interleukin-1 alpha in the cervical mucus and vaginal fluid of pregnant women with bacterial vaginosis. Am J Obstet Gynecol 169, 1161-1166 (1993).
61. Spandorfer, S. D., Neuer, A., Giraldo, P. C., Rosenwaks, Z. & Witkin, S. S. Relationship of abnormal vaginal flora, proinflammatory cytokines and idiopathic infertility in women undergoing IVF. J Reprod Med 46, 806-810 (2001).
62. Wennerholm, U. B., et al. Interleukin-1 alpha, interleukin-6 and interleukin-8 in cervico/vaginal secretion for screening of preterm birth in twin gestation. Acta Obstet Gyn Scan 77, 508-514 (1998).
63. Pellis, V., et al. Mannose binding lectin and C3 act as recognition molecules for infectious agents in the vagina. Clin Exp Immunol 139, 120-126 (2005).
64. Giraldo, P. C., et al. Mannose-binding lectin gene polymorphism, vulvovaginal candidiasis, and bacterial vaginosis. Obstet Gynecol 109, 1123-1128 (2007).
65. Kalia, N., Singh, J., Sharma, S. & Kaur, M. Impact of SNPs interplay across the locus of MBL2, between MBL and Dectin-1 gene, on women's risk of developing recurrent vulvovaginal infections. Cell Biosci 9, 35 (2019).

1. A method comprising the steps of:
(i) analysing a mucosal sample obtained from a subject to identify the presence and/or level of one or more biomarkers, or any combination of the biomarkers, wherein the biomarkers are selected from a panel of biomarkers, wherein the panel of biomarkers comprises amino-aminohexyl-diaminomethylideneamino-pentanamide, aminonaphthalene, amino-sulfanylheptenyl-amino-oxooctadecanoic acid, arachidic acid (C20:0), arginine, asparagine, aspartic acid, butynoate, cadaverine, carboxysulfanyl-pentanedioic acid, Cer(d24:1/18:1), cerotic acid (C26:0), DG(36:3), dihydroxy-octadecadienoic acid, dimethoxyphenyl-hexadecylcarbamothioyl-propenamide, docosanoic acid (C22:0), docosanylsulfanylbutanoic acid, docosenoic acid (C22:1), eicosadienoic acid (C20:2), eicosanoic acid, ethyl-hexadecylcarbamothioylamino-oxobutanoate, galabiosylceramide (d18:1/16:0), galbeta-Cer(d40:1), glutamate, glutamine, glutamyl-leucine, glycyl-phenylalanine, heptadecylenic acid (C17:1), heptadecyl-hydroxyimidazole, hexadecenylsuccinic acid, histamine, HODE, hydroxycaproic acid, hydroxyglutaric acid, hydroxyvaleric acid, lactoylcysteine, leucine, leucyl-alanine, leucyl-glutamine, leucyl-glycyl-glycine, leucyl-serine, leucyl-threonine, lignoceric acid (C24:0), linoleic acid (C18:2), linolenic acid (C18:3), lysine, lyso-PE(16:1), lyso-PE(18:1), lyso-PE(18:2), lyso-PE(20:4), lyso-PE(O-16:0), lyso-PG(16:0), lyso-PS(18:0), maltotriose, methoxymethyl methyl-glucopyranosiduronate, methyl-hexadecylcarbamothioylamino-oxobutanoate, N2-acetyl-ornithine, N-acetylcadaverine, N-acetylputrescine, nervonic acid (C24:1), nonadecanoic acid (C19:0), nonadecenoic acid (C19:1), N-palmitoyl glutamine, N-palmitoyl methionine, N-palmitoyl-cysteine, octadecanoyloxamide, octadecanyloxy-propoxy-thiazolyl-propanamide, octadecenylamino-sulfanylpropanoic acid, octadecyl-oxy-octadecanoic acid (SAHSA), oleic acid (C18:1), oxo-octadecadienoic acid, oxoproline, oxosulfanyl-oxazolidinyl-octadecanamide, oxypurinol, PC(O-14:1/18:2), pentacosanoic acid (C25:0), PG(18:1/18:2), phenyl sulfoxide, phenylalanine, phenylethylamine, phenyllactic acid, phosphatidyl glycerol, phytenic acid (C20:1), PI(20:4/18:0), propyl-hexadecylcarbamothioylamino-oxobutanoate, PS(16:0/18:1), PS(18:0/18:1), putrescine, pyridinamine, pyrrolidinecarboxaldehyde, stearic acid (18:0), styrene, succinic acid, thiomalic acid, tolualdehyde, tricosanoic acid (C23:0), tyramine, vaccenyl carnitine, valyl-alanine, valyl-glutamine, valyl-phenylalanine, valyl-serine, vinylaniline, and ximenic acid (C26:1);
(ii) forming a metabolic profile, wherein said metabolic profile is formed from data showing the presence and/or level of the biomarkers identified in step (i);
(iii) using a predictive multivariate statistical model and/or a machine learning model pre-trained with metabolic and immuno-profiling datasets obtained from a reference population to predict the subject's mucosal inflammatory state at a mucosal membrane and/or level of immune activation at a mucosal membrane; and
wherein the method further comprises the step of associating the metabolic profile of step ii) with metataxonomic data from a reference population to obtain the microbial composition of the mucosal sample.

2. (canceled).

3. The method of claim 12, wherein the biomarkers are selected from a panel of biomarkers comprising amino-aminohexyl-diaminomethylideneamino-pentanamide, aminonaphthalene, arachidic acid (C20:0), arginine, asparagine, aspartic acid, butynoate, cadaverine, carboxysulfanyl-pentanedioic acid, cerotic acid (C26:0), DG(36:3), dihydroxy-octadecadienoic acid, docosanylsulfanylbutanoic acid, eicosadienoic acid (C20:2), eicosanoic acid, glutamate, glutamine, glutamyl-leucine, glycyl-phenylalanine, heptadecylenic acid (C17:1), histamine, HODE, hydroxycaproic acid, hydroxyglutaric acid, hydroxyvaleric acid, leucine, leucyl-alanine, leucyl-glutamine, leucyl-glycyl-glycine, leucyl-serine, leucyl-threonine, lignoceric acid (C24:0), linoleic acid (C18:2), linolenic acid (C18:3), lysine, lyso-PE(16:1), lyso-PE(18:1), lyso-PE(18:2), lyso-PE(20:4), lyso-PE(O-16:0), lyso-PG(16:0), lyso-PS(18:0), maltotriose, methoxymethyl methyl-glucopyranosiduronate, N2-acetyl-ornithine, N-acetylcadaverine, N-acetylputrescine, nervonic acid (C24:1), nonadecanoic acid (C19:0), nonadecenoic acid (C19:1), N-palmitoyl methionine, N-palmitoyl-cysteine, octadecanyloxy-propoxy-thiazolyl-propanamide, octadecenylamino-sulfanylpropanoic acid, octadecyl-oxy-octadecanoic acid (SAHSA), oleic acid (C18:1), oxo-octadecadienoic acid, oxoproline, oxypurinol, PC(O-14:1/18:2), pentacosanoic acid (C25:0), PG(18:1/18:2), phenyl sulfoxide, phenylalanine, phenylethylamine, phenyllactic acid, phosphatidyl glycerol, propyl-hexadecylcarbamothioylamino-oxobutanoate, PS(16:0/18:1), PS(18:0/18:1), putrescine, pyridinamine, pyrrolidinecarboxaldehyde, stearic acid (18:0), styrene, succinic acid, thiomalic acid, tolualdehyde, tricosanoic acid (C23:0), tyramine, vaccenyl carnitine, valyl-alanine, valyl-glutamine, valyl-phenylalanine, valyl-serine, vinylaniline, and ximenic acid (C26:1), or any combinations thereof.

4.-5. (canceled).

6. The method of claim 1, wherein the prediction of the subject's mucosal inflammatory state, level of mucosal immune activation or microbial composition is based solely on the metabolic profile acquired from the mucosal membrane sample.

7. The method of claim 1, wherein the subject's mucosal inflammatory state, level of mucosal immune activation or microbial composition are obtained simultaneously.

8. The method of claim 1, wherein the immuno-profiling dataset obtained from the reference population is formed from data showing the presence or level of one or more biomarkers, wherein the biomarkers are selected from the group comprising cytokines, chemokines, immunoglobulins, growth factors, complement component molecules or any other cellular immune marker.

9. The method of claim 1, wherein the immuno-profiling dataset obtained from the reference population is formed from data showing the presence and/or level of one or more biomarkers selected from IL-1β, IL-2, IL-4, IL-5, IL-6, IL-8, IL-10, IL-18, IFN-γ, GM-CSF, TNF-α, IgA, IgG1, IgG2, IgG3, IgG4, IgM, C3b, C5, C5a, MBL, or any combinations thereof.

10. The method of claim 1, wherein the mucosal membrane is a bronchial mucosa membrane, an uterine mucosa membrane, an oesophageal mucosa membrane, a gastric mucosa membrane, an intestinal mucosa membrane, a nasal mucosa membrane, an olfactory mucosa membrane, an oral mucosa membrane, a penile mucosa membrane or a vaginal mucosa membrane.

11. The method of claim 1, wherein the mucosal sample is a swab sample, preferably-wherein said swab has been modified to enhance selectivity for any one of the selected biomarkers.

12. The method of claim 11, wherein the swab sample is a cervicovaginal swab sample or a nasal swab sample.

13. The method of claim 1, wherein the mucosal sample is analysed using mass spectrometry or wherein the mucosal sample is analysed using an ambient spectroscopy technique.

14. The method of claim 13, wherein the ambient spectroscopy technique is desorption electrospray ionisation-mass spectrometry (DESI-MS) or rapid evaporative ionization mass spectrometry (REIMS).

15. The method of claim 1, wherein the metabolic profile of the subject is obtained within 10 minutes of the sample being obtained from the subject, within 5 minutes of the sample being obtained from the subject, or within 3 minutes of the sample being obtained from the subject.

16.-17. (canceled).

18. A method of monitoring the mucosal inflammatory state, level of mucosal immune activation or microbial composition of a subject, said method comprising any feature of claim 1, wherein the subject has previously had a sample analysed for one or more biomarkers, or any combination of biomarkers, present in claim 1.

19. A method of determining a suitable course of treatment for a subject having a vaginal infection, cervical dysplasia, unsuccessful in vitro fertilization, miscarriage or pre-term birth, the method comprising predicting the inflammatory state, level of mucosal immune activation or microbial composition of a subject using the method of claim 1, wherein a suitable course of treatment is determined if the predicted inflammatory state, level of mucosal immune activation or microbial composition of the subject is indicative of vaginal infection, cervical dysplasia, unsuccessful in vitro fertilization, miscarriage or pre-term birth.

20. A method of monitoring the response of a subject to a treatment, said subject having a vaginal infection, cervical dysplasia, unsuccessful in vitro fertilization, miscarriage or pre-term birth, the method comprising predicting the inflammatory state, level of mucosal immune activation or microbial composition of a subject using the method of claim 1, wherein a suitable course of treatment is determined if the predicted inflammatory state, level of mucosal immune activation or microbial composition of the subject is indicative of vaginal infection, cervical dysplasia, unsuccessful in vitro fertilization, miscarriage and/or pre-term birth.

21. (canceled).

22. A method of treating a vaginal infection in a subject, said method comprising the steps of:
(i) analysing a mucosal sample obtained from the subject to identify the presence or level of one or more biomarkers, or any combination of biomarkers, wherein the biomarkers are selected from a panel of biomarkers, wherein the panel of biomarkers comprises amino-aminohexyl-diaminomethylideneamino-pentanamide, aminonaphthalene, amino-sulfanylheptenyl-amino-oxooctadecanoic acid, arachidic acid (C20:0), arginine, asparagine, aspartic acid, butynoate, cadaverine, carboxysulfanyl-pentanedioic acid, Cer(d24:1/18:1), cerotic acid (C26:0), DG(36:3), dihydroxy-octadecadienoic acid, dimethoxyphenyl-hexadecylcarbamothioyl-propenamide, docosanoic acid (C22:0), docosanylsulfanylbutanoic acid, docosenoic acid (C22:1), eicosadienoic acid (C20:2), eicosanoic acid, ethyl-hexadecylcarbamothioylamino-oxobutanoate, galabiosylceramide (d18:1/16:0), galbeta-Cer(d40:1), glutamate, glutamine, glutamyl-leucine, glycyl-phenylalanine, heptadecylenic acid (C17:1), heptadecyl-hydroxyimidazole, hexadecenylsuccinic acid, histamine, HODE, hydroxycaproic acid, hydroxyglutaric acid, hydroxyvaleric acid, lactoylcysteine, leucine, leucyl-alanine, leucyl-glutamine, leucyl-glycyl-glycine, leucyl-serine, leucyl-threonine, lignoceric acid (C24:0), linoleic acid (C18:2), linolenic acid (C18:3), lysine, lyso-PE(16:1), lyso-PE(18:1), lyso-PE(18:2), lyso-PE(20:4), lyso-PE(O-16:0), lyso-PG(16:0), lyso-PS(18:0), maltotriose, methoxymethyl methyl-glucopyranosiduronate, methyl-hexadecylcarbamothioylamino-oxobutanoate, N2-acetyl-ornithine, N-acetylcadaverine, N-acetylputrescine, nervonic acid (C24:1), nonadecanoic acid (C19:0), nonadecenoic acid (C19:1), N-palmitoyl glutamine, N-palmitoyl methionine, N-palmitoyl-cysteine, octadecanoyloxamide, octadecanyloxy-propoxy-thiazolyl-propanamide, octadecenylamino-sulfanylpropanoic acid, octadecyl-oxy-octadecanoic acid (SAHSA), oleic acid (C18:1), oxo-octadecadienoic acid, oxoproline, oxosulfanyl-oxazolidinyl-octadecanamide, oxypurinol, PC(O-14:1/18:2), pentacosanoic acid (C25:0), PG(18:1/18:2), phenyl sulfoxide, phenylalanine, phenylethylamine, phenyllactic acid, phosphatidyl glycerol, phytenic acid (C20:1), PI(20:4/18:0), propyl-hexadecylcarbamothioylamino-oxobutanoate, PS(16:0/18:1), PS(18:0/18:1), putrescine, pyridinamine, pyrrolidinecarboxaldehyde, stearic acid (18:0), styrene, succinic acid, thiomalic acid, tolualdehyde, tricosanoic acid (C23:0), tyramine, vaccenyl carnitine, valyl-alanine, valyl-glutamine, valyl-phenylalanine, valyl-serine, vinylaniline, and ximenic acid (C26:1);
(ii) forming a metabolic profile, wherein said metabolic profile is formed from data showing the presence or level of the biomarkers identified in step (i);
(iii) using a predictive multivariate statistical model or a machine learning model pre-trained with metabolic and immuno-profiling datasets obtained from a reference population to predict the subject's mucosal inflammatory state or level of mucosal immune activation; and
wherein the method further comprises the step of associating the metabolic profile of step ii) with metataxonomic data from a reference population to obtain the microbial composition of the mucosal sample, wherein when the subject's mucosal inflammatory state, level of mucosal immune activation or microbial composition are indicative of a vaginal infection, the subject is administered an anti-inflammatory agent, an antibiotic, a live biotherapeutic, a prebiotic, a probiotic, progesterone (P4) or undergoes a cervical cerclage procedure.

23. The method of claim 18, wherein the vaginal infection is a bacterial infection, a viral infection, a yeast infection and/or a sexually transmitted infection, preferably wherein the vaginal infection is a bacterial vaginosis infection, a candidiasis infection, a human papillomavirus (HPV) infection, a herpes simplex virus (HSV) infection or a human immunodeficiency virus (HIV) infection.

24. A method of preventing pre-term birth in a subject, said method comprising the steps of:
(i) analysing a mucosal sample obtained from the subject to identify the presence and/or level of one or more biomarkers, or any combination of biomarkers, wherein the biomarkers are selected from a panel of biomarkers, wherein the panel of biomarkers comprises amino-aminohexyl-diaminomethylideneamino-pentanamide, aminonaphthalene, amino-sulfanylheptenyl-amino-oxooctadecanoic acid, arachidic acid (C20:0), arginine, asparagine, aspartic acid, butynoate, cadaverine, carboxysulfanyl-pentanedioic acid, Cer(d24:1/18:1), cerotic acid (C26:0), DG(36:3), dihydroxy-octadecadienoic acid, dimethoxyphenyl-hexadecylcarbamothioyl-propenamide, docosanoic acid (C22:0), docosanylsulfanylbutanoic acid, docosenoic acid (C22:1), eicosadienoic acid (C20:2), eicosanoic acid, ethyl-hexadecylcarbamothioylamino-oxobutanoate, galabiosylceramide (d18:1/16:0), galbeta-Cer (d40:1), glutamate, glutamine, glutamyl-leucine, glycyl-phenylalanine, heptadecylenic acid (C17:1), heptadecyl-hydroxyimidazole, hexadecenylsuccinic acid, histamine, HODE, hydroxycaproic acid, hydroxyglutaric acid, hydroxyvaleric acid, lactoylcysteine, leucine, leucyl-alanine, leucyl-glutamine, leucyl-glycyl-glycine, leucyl-serine, leucyl-threonine, lignoceric acid (C24:0), linoleic acid (C18:2), linolenic acid (C18:3), lysine, lyso-PE(16:1), lyso-PE(18:1), lyso-PE(18:2), lyso-PE(20:4), lyso-PE(O-16:0), lyso-PG(16:0), lyso-PS(18:0), maltotriose, methoxymethyl methyl-glucopyranosiduronate, methyl-hexadecylcarbamothioylamino-oxobutanoate, N2-acetyl-ornithine, N-acetylcadaverine, N-acetylputrescine, nervonic acid (C24:1), nonadecanoic acid (C19:0), nonadecenoic acid (C19:1), N-palmitoyl glutamine, N-palmitoyl methionine, N-palmitoyl-cysteine, octadecanoyloxamide, octadecanyloxy-propoxy-thiazolyl-propanamide, octadecenylamino-sulfanylpropanoic acid, octadecyl-oxy-octadecanoic acid (SAHSA), oleic acid (C18:1), oxo-octadecadienoic acid, oxoproline, oxosulfanyl-oxazolidinyl-octadecanamide, oxypurinol, PC(O-14:1/18:2), pentacosanoic acid (C25:0), PG(18:1/18:2), phenyl sulfoxide, phenylalanine, phenylethylamine, phenyllactic acid, phosphatidyl glycerol, phytenic acid (C20:1), PI(20:4/18:0), propyl-hexadecylcarbamothioylamino-oxobutanoate, PS(16:0/18:1), PS(18:0/18:1), putrescine, pyridinamine, pyrrolidinecarboxaldehyde, stearic acid (18:0), styrene, succinic acid, thiomalic acid, tolualdehyde, tricosanoic acid (C23:0), tyramine, vaccenyl carnitine, valyl-alanine, valyl-glutamine, valyl-phenylalanine, valyl-serine, vinylaniline, and ximenic acid (C26:1);
(ii) forming a metabolic profile, wherein said metabolic profile is formed from data showing the presence or level of the biomarkers identified in step (i):
(iii) using a predictive multivariate statistical model or a machine learning model pre-trained with metabolic and immuno-profiling datasets obtained from a reference population to predict the subject's mucosal inflammatory state and/or level of mucosal immune activation; and
wherein the method further comprises the step of associating the metabolic profile of step ii) with metataxonomic data from a reference population to obtain the microbial composition of the mucosal sample, wherein when the subject's mucosal inflammatory state, level of mucosal immune activation or microbial composition are indicative of pre-term birth, the subject is administered a preventative therapy or undergoes a preventative procedure.

25. The method of claim 24, wherein the preventative therapy is progesterone (P4), and the preventative procedure is a cervical cerclage procedure.

26. A kit comprising
a first device arranged and adapted to direct a spray of charged droplets onto a surface of a swab in order to generate a plurality of analyte ions:
a second device arranged and adapted to analyse said analyte ions, wherein said analyte ions are analysed for the presence or level of one or more biomarkers, or any combination of biomarkers, wherein the biomarkers are selected from the panel of biomarkers of claim 1.


SEQUENCE LISTING (11 sequences)

SEQ ID NO: 1 - DNA, 29 bp, synthetic construct
  misc_feature 1..29: note = Forward Primer (Illumina i5 Adapter)
  source 1..29: mol_type = other DNA
aatgatacggcgaccaccgagatctacac

SEQ ID NO: 2 - DNA, 10 bp, synthetic construct
  misc_feature 1..10: note = Forward Primer (Primer Pad)
  source 1..10: mol_type = other DNA
tatggtaatt

SEQ ID NO: 3 - DNA, 19 bp, synthetic construct
  misc_feature 1..19: note = Forward Primer (28F Primer)
  misc_difference 11: note = n is a, c, g, or t
  source 1..19: mol_type = other DNA
gagtttgatcntggctcag

SEQ ID NO: 4 - DNA, 24 bp, synthetic construct
  misc_feature 1..24: note = Reverse Fusion Primer (Illumina i7 adapter)
  source 1..24: mol_type = other DNA
caagcagaagacggcatacgagat

SEQ ID NO: 5 - DNA, 10 bp, synthetic construct
  misc_feature 1..10: note = Reverse Fusion Primer (Primer Pad)
  source 1..10: mol_type = other DNA
agtcagtcag

SEQ ID NO: 6 - DNA, 18 bp, synthetic construct
  misc_feature 1..18: note = Reverse Fusion Primer (519R Primer)
  misc_difference order(3,8): note = n is a, c, g, or t
  source 1..18: mol_type = other DNA
gtnttacngcggckgctg

SEQ ID NO: 7 - DNA, 19 bp, synthetic construct
  misc_feature 1..19: note = Forward Primer (28F-Borrellia)
  source 1..19: mol_type = other DNA
gagtttgatcctggcttag

SEQ ID NO: 8 - DNA, 19 bp, synthetic construct
  misc_feature 1..19: note = Forward Primer (28F-Chlorflex)
  source 1..19: mol_type = other DNA
gaatttgatcttggttcag

SEQ ID NO: 9 - DNA, 19 bp, synthetic construct
  misc_feature 1..19: note = Forward Primer (28F-Bifido)
  source 1..19: mol_type = other DNA
gggttcgattctggctcag

SEQ ID NO: 10 - DNA, 19 bp, synthetic construct
  misc_feature 1..19: note = Forward Primer (28F)
  misc_difference 11: note = n is a, c, g, or t
  source 1..19: mol_type = other DNA
gagtttgatcntggctcag

SEQ ID NO: 11 - DNA, 19 bp, synthetic construct
  misc_feature 1..19: note = Reverse Primer (388R)
  source 1..19: mol_type = other DNA
tgctgcctcccgtaggagt